(19) United States
(12) Patent Application Publication
(10) Pub. No.: US 2018/0233237 A1
(43) Pub. Date: Aug. 16, 2018

(54) APPARATUS AND METHOD FOR PROCESSING AND/OR PROVIDING HEALTHCARE INFORMATION AND/OR HEALTHCARE-RELATED INFORMATION WITH OR USING AN ELECTRONIC HEALTHCARE RECORD AND INFORMATION REGARDING AND/OR OBTAINED WITH OR FROM ELECTRONIC INTERACTIVE ACTIVITY, INFORMATION, CONTENT, OR MEDIA

(71) Applicants: RAYMOND ANTHONY JOAO, YONKERS, NY (US); STEVEN WHITEFIELD RITCHESON, MARINA DEL REY, CA (US)

(72) Inventors: RAYMOND ANTHONY JOAO, YONKERS, NY (US); STEVEN WHITEFIELD RITCHESON, MARINA DEL REY, CA (US)

(21) Appl. No.: 15/951,205

(22) Filed: Apr. 12, 2018

Related U.S. Application Data
(63) Continuation of application No. 14/550,530, filed on Nov. 21, 2014.
(60) Provisional application No. 61/914,290, filed on Dec. 10, 2013.

(51) Int. Cl. G16H 80/00; G16H 10/60; G16H 50/20; G16H 50/70

(52) U.S. Cl. CPC G16H 80/00; G16H 50/70; G16H 50/20; G16H 10/60

(57) ABSTRACT

An apparatus, including a user device, provider device, and computer. The computer processes a request to access a video game transmitted from the user device. The video game is accessed via an electronic healthcare record of the individual. The computer administers or provides the video game to the individual via the user device. The computer records and stores, in the electronic healthcare record, information regarding the individual's activity during, or interactions with, the video game. The computer provides the video game to the provider device for allowing the provider to observe or monitor the individual's actions or interactions in or during the video game via a second display. Notes or observations entered into the provider device, regarding the individual or actions of the individual during the video game, are transmitted from the provider device to the computer, and are stored in the electronic healthcare record.

### RELATED APPLICATIONS

This application is a continuation application of U.S. patent application Ser. No. 14/550,530, filed Nov. 21, 2014, and entitled “APPARATUS AND METHOD FOR PROCESSING AND/OR PROVIDING HEALTHCARE INFORMATION AND/OR HEALTHCARE-RELATED INFORMATION WITH OR USING AN ELECTRONIC HEALTHCARE RECORD AND INFORMATION REGARDING AND/OR OBTAINED WITH OR FROM ELECTRONIC INTERACTIVE ACTIVITY, INFORMATION, CONTENT, OR MEDIA”, the subject matter and teachings of which are hereby incorporated by reference herein in their entirety. U.S. patent application Ser. No. 14/550,530 claims the benefit of the priority of U.S. Provisional Patent Application Ser. No. 61/914,290, filed Dec. 10, 2013, and entitled “APPARATUS AND METHOD FOR PROCESSING AND/OR PROVIDING HEALTHCARE INFORMATION AND/OR HEALTHCARE-RELATED INFORMATION WITH OR USING AN ELECTRONIC HEALTHCARE RECORD AND INFORMATION REGARDING AND/OR OBTAINED WITH OR FROM ELECTRONIC INTERACTIVE ACTIVITY, INFORMATION, CONTENT, OR MEDIA”, the subject matter and teachings of which are hereby incorporated by reference herein in their entirety.

### FIELD OF THE INVENTION

The present invention pertains to an apparatus and a method for processing and/or for providing healthcare information and/or healthcare-related information with or using an electronic healthcare record and information regarding and/or obtained with or from electronic interactive activity, information, content, or media, and, in particular, to an apparatus and a method for processing and/or for providing healthcare information and/or healthcare-related information with or utilizing an electronic healthcare record and information regarding and/or obtained with or from electronic interactive activity, information, content, or media, and any data and/or information contained therein for a number and variety of applications.

### BACKGROUND OF THE INVENTION

In the field of healthcare, video games have recently gained notoriety for being utilized to treat certain individuals afflicted with illnesses or conditions. With more and more people gaining access to the Internet and/or the World Wide Web and gaining access to personal computers, laptop computers, notebook computers, tablet computers, tablets, cellular telephones, smart phones, and like devices, coupled with the lighting fast pace that new software applications and video games are being developed for use with such devices, there is no doubt that video games have the potential to find ever-increasing uses in treating and/or diagnosis certain healthcare illnesses, sicknesses, injuries, conditions, syndromes, states of dysfunction, and/or any healthcare or healthcare-related problems or impairments, for individuals and/or patients of all ages.

With the potential for video games to find more important and more widespread uses in treating and/or in diagnosing healthcare illnesses, sicknesses, injuries, conditions, syndromes, states of dysfunction, and/or any healthcare or healthcare-related problems or impairments, there exists a need for managing the identification or selection of these video games, a need for making these video games available to individuals or patients in a convenient and efficient manner, a need for monitoring and/or observing an individual's or a patient's performance in playing these video games, and a need for efficiently integrating the use of video games in managing the healthcare needs of an individual or a patient.

In view of the foregoing, there is a need for an apparatus and a method which can incorporate and/or integrate the use of video games and any other electronic interactive activities into various healthcare activities and healthcare-related activities, including, but not limited to, treatment planning, treatment monitoring, diagnostic procedures or processes, therapy activities or exercises, rehabilitation activities or exercises, training activities or exercises, or retraining activities or exercises, which overcomes the shortcomings of the prior art.

### SUMMARY OF THE INVENTION

The present invention pertains to an apparatus and a method for processing and/or for providing healthcare information and/or healthcare-related information with or using an electronic healthcare record and information regarding and/or obtained with or from electronic interactive activity, information, content, or media, and, in particular, to an apparatus and a method for processing and/or for providing healthcare information and/or healthcare-related information with or utilizing an electronic healthcare record and information regarding and/or obtained with or from electronic interactive activity, information, content, or media, and any data and/or information contained therein for a number and variety of applications, which overcomes the shortcomings of the prior art.

The present invention provides an apparatus and method which can be utilized in order to facilitate the use of an electronic interactive activity or electronic interactive activities in performing a diagnostic evaluation and/or analysis of an individual or a patient, in treating an individual or a patient, in treating an individual or a patient pursuant to a treatment, a course of treatment, or a treatment plan, as part of a therapy or a therapy regimen, as part of physical therapy or a physical therapy regimen, as part of mental or behavioral therapy or a mental or behavioral therapy regimen, as party of a rehabilitation regimen, as part of a training regimen, as part of a re-training regimen, or for any healthcare or healthcare-related activity or purpose.

The present invention can be utilized in order to provide an electronic interactive activity or electronic interactive activities via an electronic healthcare record or an electronic healthcare file and can be utilized by individuals and patients of any and/or all ages or age groups in order to provide for the use of an electronic interactive activity or electronic interactive activities which can be utilized in performing diagnostic evaluations or analyses, diagnoses, treatments, treatment planning, therapy, rehabilitation, training, or re-training, for and/or regarding any illnesses, sicknesses, injuries, conditions, syndromes, states of dysfunction, and/or any healthcare or healthcare-related problems or impairments, including, but not limited to, developmental, cognitive, physical, mental, behavioral, motor, speech, hearing, seeing, learning, or any other, disorders, illnesses, sicknesses, injuries, conditions, syndromes, or states of dysfunction, Alzheimer's disease, Dementia, learning disorders, attention deficit disorders, learning disabilities, muscular coordination disorders, chronic pain conditions, developmental disorders, speech disorders, cognitive disorders, mental acuity disorders, spatial disorders, temporal disorders, psychological disorders, physiological disorders, post traumatic stress disorders, lazy eye, atrophies of any kind or type, conditions or disorders that affect spatial and/or temporal conditions, muscular coordination and/or any other type of coordination or activity, or any other healthcare or healthcare-related disorder, illness, sickness, injury, condition, syndrome, or state of dysfunction.

The electronic interactive activity or electronic interactive activities can be or can include any type or kind of video game or video games, audio game or audio games, video and audio game or video and audio games, electronic or digital exercise or exercises, electronic or digital puzzle or puzzles or activity or activities, electronic or digital diagnostic activity or activities or tool or tools, electronic or digital treatment, treating planning, or therapy, activity or activities or tool or tools, electronic or digital mental health or behavioral health activity or activities or tool or tools, electronic or digital physical or physical therapy activity or activities or tool or tools, electronic or digital training activity or activities or tool or tools, electronic or digital rehabilitation activity or activities or tool or tools, electronic or digital mental health or behavioral health training or re-training activity or activities or tool or tools, electronic or digital physical training or re-training activity or activities or tool or tools, survey or surveys, questionnaire or questionnaires, or any other game or games, exercise or exercises, activity or activities, information gathering event or events or occurrence or occurrences, or any other content or media which can be utilized in connection with a computer, a computer system, a communication device, or any other electronic device, in order to obtain any data and/or information from an individual or patient, and/or which can be used in performing a diagnosis or a diagnostic evaluation or analysis, in identifying a treatment, a course of treatment, or a treatment plan, or in administering a therapy, rehabilitation activities, training activities, re-training activities, or which can be used in any other manner in delivering or providing healthcare services or healthcare-related services.

The apparatus of the present invention includes a central processing computer or central processing computer system which can be a network or server computer. The apparatus also includes a healthcare provider communication device or computer which is associated with a healthcare provider such as a healthcare professional, a hospital, a clinic, and/or any other provider of services described herein. The healthcare provider computer(s) can communicate with, and operate in conjunction with, the central processing computer and/or any of the other computers and/or computer systems or communication devices described herein.

The apparatus can also include a healthcare payer communication device or computer which is associated with a healthcare payer such as a healthcare insurer, insurance company, health maintenance organization, a clinic, and/or any other payer of healthcare services and products described herein. The healthcare payer computer(s) can communicate with, and operate in conjunction with, central processing computer and/or any of the other computers and/or computer systems or communication devices described herein.

The apparatus can also include a user or patient communication device or computer which is associated with an individual or patient, or a caregiver of the individual or patient, who seeks or who is provided with healthcare and/or related services, products and/or related information. The user or patient communication device(s) can communicate with, and operate in conjunction with, central processing computer and/or any of the other computers and/or computer systems described herein.

The apparatus can also include an intermediary communication device or computer which is associated with an intermediary, a broker, an agent, and/or any other individual and/or entity, or any third party, that can utilize the present invention in order to act for and/or on behalf of any other individual, party, or entity, described herein. The intermediary computer(s) can communicate with, and operate in conjunction with, central processing computer and any of the other computers and/or computer systems described herein.

The apparatus can also include a healthcare records computer which can be or can include a computer or computer system, or any number of computers or computer systems, or a cloud computer system or cloud system. The healthcare records computer can serve to store and house an electronic healthcare record or files or any number of electronic healthcare records or files. The healthcare records computer can be associated with any provider, insurer, payer, intermediary, insurance exchange, or any user, individual, patient, organization, or entity, who or which utilizes the present invention.

Each healthcare records computer can be utilized to store an electronic healthcare record or file or any number of electronic healthcare records or files which can be accessed by the central processing computer, by any provider communication device, by any insurer or payer communication device, by any user or patient communication device, by any intermediary communication device, or by any other computer, communication device or other device described herein as being utilized in connection with the present invention. The healthcare records computer can also be utilized to facilitate cloud storage of any electronic healthcare record(s) or file(s).

The apparatus can also include an electronic interactive content/media computer which can be or can include a computer or computer system, or any number of computers or computer systems, or a cloud computer system or cloud system. The electronic interactive content/media computer can serve to store and house any electronic interactive activity or any number of kinds or types of electronic interactive activities which can be provided by, or which can be utilized in connection with, the present invention. The electronic interactive content/media computer can be associated with any provider, supplier, or vendor, of any electronic interactive activity or electronic interactive activities.

Each electronic interactive content/media computer can be utilized to store any electronic interactive activity or electronic interactive activities which can be accessed by the central processing computer, by any provider communication device, by any insurer or payer communication device, by any user or patient communication device, by any intermediary communication device, or by any other computer, communication device or other device described herein as being utilized in connection with the present invention. The electronic interactive content/media computer can also be utilized to facilitate cloud storage of any electronic interactive activity or electronic interactive activities.

The apparatus can also include an insurance exchange computer, or any number of insurance exchange computers which can be utilized to process and store information regarding the selling of healthcare insurance, disability insurance, and life insurance, policies, products, and/or services, to any of the herein-described users, individuals, patients, or entities, who or which utilize the apparatus and method of the present invention. The insurance exchange computer can be utilized to advertise, provide information regarding, sell, and/or maintain records regarding, and process any other information regarding, group insurance as well as individual or family insurance policies, products, or services. The insurance exchange computer can also be utilized to sell automobile, homeowners, business, and/or liability insurance policies, products, or services.

The apparatus can also include a social networking computer. The social networking computer can be linked with, and utilized in connection with, the apparatus so as to allow and/or facilitate integrating the apparatus of the present invention with social networks, social networking, and social media. The social networking computer can be associated with a social networking company, a social networking website, or social networking entity, website, group, organization, or association. The social networking computer can also be associated with any one or any number of social networking companies, social networking websites, or social networking entities, websites, groups, organizations, or associations. The social networking computer can also provide links to any computers associated with any one or any number of social networking companies, social networking websites, or social networking entities, websites, groups, organizations, or associations. The social networking computer can perform any and all of the functions performed by any social networking company, a social networking website, or social networking entity, website, group, organization, or association. Any number of social networking computers can be utilized in connection with the present invention.

The apparatus can also include a media computer. The media computer can provide, and be a source of, news information, current events information, healthcare information, healthcare or healthcare-related news, current events, advertisements, and/or marketing information or materials, which can be disseminated or distributed via the present invention. Any number of media computers, with each being dedicated to providing any number, types, or kinds of, news information, current events information, healthcare information, healthcare or healthcare-related news, current events, advertisements, and/or marketing information or materials, can be utilized in connection with the present invention.

Each of the central processing computer(s), the provider communications devices, the payer communication devices, the user or patient communication devices, the intermediary communication devices, the healthcare records computers, the electronic interactive content/media computers, the insurance exchange computers, the social networking computers, and the media computers can communication in a bi-directional manner with, and/or can send and/or receive signals, messages, reports, notification messages, alerts, or any other communications or electronic communication transmissions, to, from and/or between, any other, or any number of, other central processing computer(s), if utilized, provider communications devices, payer communication devices, user or patient communication devices, intermediary communication devices, healthcare records computers, electronic interactive content/media computers, insurance exchange computers, social networking computers, and/or the media computers.

Each of the central processing computer(s), the provider communications devices, the payer communication devices, the user or patient communication devices, the intermediary communication devices, the healthcare records computers, the electronic interactive content/media computers, the insurance exchange computers, the social networking computers, and the media computers can be linked to or with any other central processing computer(s), if utilized, provider communications devices, payer communication devices, user or patient communication devices, intermediary communication devices, healthcare records computers, electronic interactive content/media computers, insurance exchange computers, social networking computers, and/or the media computers via a wired link or line or a wireless link.

Each of the provider communications devices, payer communication devices, user or patient communication devices, intermediary communication devices, healthcare records computers, electronic interactive content/media computers, insurance exchange computers, social networking computers, and/or the media computers can be connected with or linked with the central processing computer.

Any and/or all of the signals, messages, reports, notification messages, or any other communications, described herein as being transmitted from one device, computer, or communication device, to another, can be, or can be included in, or be attached to, an e-mail message, an instant messaging message, an electronic transmission, or an electronic data transmission or electronic data interchange, or can be transmitted via any other data or information transmission, and can be transmitted via or using any appropriate or necessary computer(s) or device(s).

The present invention can be utilized on, and/or over, the Internet and/or the World Wide Web and/or any other communication network, telecommunication network, telephone network, a line-connected network, or a wireless communication network, or any combination of same. The present invention can also utilize wireless Internet and/or World Wide Web services, equipment and/or devices. The central processing computer(s) an/or any of the herein-described computers or communication devices can have a web site or web sites associated with same.

The present invention can be utilized on, or over, the Internet and/or the World Wide Web and/or on, or over, any other communication network or system, including, but not limited to, a communication network or system, a telecommunication network or system, a telephone communication network or system, a cellular communication network or system, a wireless communication network or system, a wireless Internet network or system, a wireless World Wide Web network or system, a line or wired communication network or system, a digital communication network or system, a personal communication network or system, a personal communication services (PCS) network or system, a satellite communication network or system, a broad band communication network or system, a low earth orbiting (LEO) satellite network or system, a public switched telephone network or system, a telephone communication network or system, a radio communication network or system, and/or any other communication network or system, and/or any combination of the above communication networks or systems.

The apparatus and method of the present invention can utilize electronic commerce technologies and security methods, techniques and technologies, in any and/or all of the instances of data and/or information processing, and/or data and/or information transmission described herein.

Each of the central processing computer(s), as well as each of the herein-described computers or communication devices can include a central processing unit or CPU, a random access memory device(s) (RAM), a read only memory device(s), and a user input device. Each of the central processing computer(s), as well as each of the herein-described computers or communication devices can also include a display device, a transmitter(s), a receiver, a database(s), and an output device. The database(s) can contain any and/or all of the data and or information which is needed to perform the various processing methods, services, functions and/or operations, described herein.

The present invention can be utilized in numerous preferred embodiments in order to provide a vast array of healthcare and healthcare-related services for any one or more of the various parties described herein.

In instances where an individual's or patient's healthcare-related records, healthcare or healthcare-related files, or healthcare or healthcare-related histories, are stored and/or located in different or separate electronic healthcare records, electronic medical records, electronic dental records, electronic pharmacy records, and/or electronic behavioral healthcare records, or stored or located on different or separate computers or computer systems, including, but not limited to, the central processing computer, healthcare records computers, or any other computers or computer systems, the apparatus of the present invention or the central processing computer can also contain a master individual or patient index containing, for each individual or patient, the individual's or patient's name, address, social security number or other uniquely assigned identification number or identifying data or information, mother's maiden name, name of spouse or partner, name(s) of child, children or others for who the individual or patient is responsible for providing care legally or otherwise, each electronic healthcare records, electronic medical records, electronic dental records, electronic pharmacy records, and/or electronic behavioral healthcare records, containing data and/or information regarding the individual or patient, or in or on which the individual or patient has a record, file, history, or entry, and/or a link(s) or hyperlink(s) to each electronic healthcare records, electronic medical records, electronic dental records, electronic pharmacy records, and/or electronic behavioral healthcare records, containing data and/or information regarding the individual or patient, or in or on which the individual or patient has a record, file, history, or entry.

The present invention can be utilized in order to process information and/or to provide information regarding a diagnosis or possible diagnoses for or regarding an individual or a patient, to process and/or to provide diagnostic information based on a symptom or symptoms, an observation or observations, an examination finding or examination findings, any data and/or information obtained from a procedure performed on or with an individual or a patient or obtained from or during an examination, a test, a procedure, or an analysis, or any data and/or information obtained from or with any electronic interactive activity or electronic interactive activities described herein.

The present invention can also be utilized in order to process information and/or to provide information regarding a treatment, a prescribed treatment, or possible or available treatments, as well as any treatment plan or treatment plans associated with same, for an individual or a patient, which can include processing and/or providing data and/or information for or associated with identifying, prescribing, providing, administering, monitoring, recoding, and/or processing any data and/or information obtained from or with, any electronic interactive activity or electronic interactive activities which can be utilized in a treatment or an treatment plan for the individual or the patient.

The present invention can also provide for automatic claim form generation and submission or request for payment form generation and submission via the central processing computer once a final diagnosis and/or a treatment has been prescribed by a healthcare provider, and/or upon the occurrence of an examination and/or the administration of a treatment, or the performance of a procedure.

The present invention can also be utilized to provide information regarding any electronic interactive activity or electronic interactive activities with or via an electronic healthcare record, provide access to an electronic interactive activity, provide or administer an electronic interactive activity or electronic interactive activities from or via an electronic healthcare record, provide for the monitoring and/or the recording of data and/or information, as well as any results obtained from and/or recorded, from an administration of an electronic interactive activity or electronic interactive activities, with, by, or using, an electronic healthcare record, or to utilize data and/or information obtained from an electronic interactive activity or electronic interactive activities, in performing follow-up diagnostic processing, treatment planning and/or treatment plan monitoring, and/or any other healthcare information processing and/or healthcare-related processing for or regarding an individual or a patient.

The apparatus and method of the present invention can also be utilized to process data and/or information obtained with or from an electronic interactive activity or electronic interactive activities, provided from or via an electronic healthcare record in performing a diagnosis, in prescribing a treatment plan, in monitoring a treatment plan, in processing information for evaluating a treatment plan, in processing information for or regarding evaluating an individual's or a patient's progress or response to a treatment or a treatment plan, the effectiveness of the treatment or the treatment plan, or the progress or effectiveness of the treatment or treatment plan in meeting or achieving goals related to curing, healing, rehabilitating, training, or re-training, an individual or patient.

The apparatus and method of the present invention can also be utilized to provide and/or administer an electronic interactive activity, record and process data and/or information regarding an electronic interactive activity performed by an individual or a patient, allow for a healthcare provider or any other provider to monitor and/or observe the individual's or the patient's activities in performing an electronic interactive activity, allow for a healthcare provider or any other provider to monitor and/or observe the individual's or the patient's activities in performing an electronic interactive activity in real-time and/or otherwise, provide a recording of the individual's or the patient's activities in performing an electronic interactive activity to a healthcare provider or any other provider, and/or allow for a plurality or individual or patient's to engage in groups activities with or using an electronic interactive activity or electronic interactive activities.

The present invention can also be utilized in order to allow an individual or a patient to access any electronic interactive activity prescribed for him or her and to engage in same, use same, perform same, or participate in same, via his or her electronic healthcare record. Any data and/or information regarding the individual's or the patient's accessing of the electronic interactive activity prescribed for him or her, including but not limited to, the date and time of same, and any data and/or information regarding the individual's or the patient's engagement in or with same, use of same, performance of same, or participation in same, including but not limited to, any information provided by the individual or the patient, any information obtained by the apparatus or the central processing computer, any information obtained by the apparatus or the central processing computer in monitoring and/or observing the individual's or the patient's engagement with, use of, performance of, or participation in, the electronic interactive activity, and/or any performance results, measurements, or scores, the individual to the patient has achieved in the engagement with, use of, performance of, or participation in, the electronic interactive activity, can be stored by the apparatus, the central processing computer, and/or the user or patient communication device used by the individual or the patient.

The present invention can also be utilized in order to allow for group participation by a plurality of individuals or patients in an electronic interactive activity or to allow a group of individuals or patients to access and engaged in, use, perform, or participate in, a same electronic interactive activity with each other and/or at the same time. For example, any group participants can be identified from any social network(s) or social networking groups of individuals or patients who may either have, be suspected of having, or be under the observation or care for or regarding, a same illness, sickness, injury, condition, syndrome, state of dysfunction, and/or any healthcare or healthcare-related problem or impairment.

The present invention can also be utilized to allow any healthcare provider of the individual or patient, or of any other individual or patient who engages in, uses, performs, or participates in, the electronic interactive activity section, can access the electronic interactive activity section via his or her provider communication device and can monitor and/or observe the activity of the individual or the patient. The healthcare provider can also make and/or enter, into the individual's or the patient's electronic healthcare record or electronic healthcare file, any notes, comments, or observations, regarding the individual or the patient, or any notes, comments, or observations, about or regarding the individual's or the patient's actions, engagement, use, performance, or participation, in or during the electronic interactive activity section at any time before, during, or after, the electronic interactive activity section.

In the case where a plurality of individuals or patients engage in, use, perform, or participate in, the electronic interactive activity section, a healthcare provider of or for each individual or patient can access the electronic interactive activity section and monitor and/or observe the activity of the individual or patient he or she seeks to monitor and/or observe and/or to monitor and/or observe the activity of the individual or the patient under his or her care. In such an embodiment, any healthcare provider can also make and/or enter, into any individual's or patient's electronic healthcare record or electronic healthcare file, any notes, comments, or observations, regarding that individual or patient, or any notes, comments, or observations, about or regarding that individual's or patient's actions, engagement, use, performance, or participation, in or during the electronic interactive activity section at any time before, during, or after, the electronic interactive activity section. In this manner, any notes, comments, or observations, made by any other healthcare provider, other than the healthcare provider of the individual or the patient, can be considered and/or utilized in diagnosing or treating the individual or the patient.

The present invention can also be utilized by a healthcare provider of the individual or the patient to access and/or to review, via the individual's or the patient's electronic healthcare record or electronic healthcare file, any data and/or information regarding or obtained from or during any electronic interactive activity session or any number of electronic activity sessions in which the individual or the patient was involved and/or any performance results, measurements, or scores, of or achieved by the individual or the patient regarding or relating to same, to make or enter any notes, comments, or observations, regarding same, to perform an updated diagnostic evaluation of the individual or the patient, and/or to generate an updated diagnostic report, treatment report, or treatment plan(s), for the individual or the patient, based on or using any of the data and/or information regarding or obtained from or during any electronic interactive activity session in which the individual or the patient was involved or any notes, comments, or observations, made or provided by the healthcare provider.

The present invention can be utilized in conjunction with electronic interactive activities of any type or kind in order to diagnose and/or to treat illnesses, sicknesses, injuries, conditions, syndromes, states of dysfunction, and/or any healthcare or healthcare-related problems or impairments, for individuals or patients of all ages.

The present invention can be utilized to automatically generate an electronic interactive activity message(s), containing information regarding an availability of an electronic interactive activity or of an availability of an update to an electronic interactive activity, and to transmit same to a provider communication device of or associated with a provider of an individual or patient, to the a payer communication device of or associated with an insurer or a payer of an individual of or a patient, to a user or patient communication device of or associated with an individual or a patient, or a caregiver of the individual or the patient, or to an intermediary communication device of or associated with any intermediary.

The present invention can be utilized to automatically generate a prescribed or recommended electronic interactive activity reminder message(s) containing information for reminding an individual or a patient, or his or her caregiver, or his or her provider, to access, engage in, use, perform, or participate in, any prescribed or recommended electronic interactive activity, and to transmit same to a provider communication device of or associated with a provider of an individual or patient, to the a payer communication device of or associated with an insurer or a payer of an individual of or a patient, to a user or patient communication device of or associated with an individual or a patient, or a caregiver of the individual or the patient, or to an intermediary communication device of or associated with any intermediary.

Any of the herein-described reports, plans, messages, or data and/or information, described herein as being transmitted from one computer or communication device to another computer or communication device, can be transmitted as, in, or attached to, an e-mail message, a text message, an SMS message, or any electronic transmission or electronic data transmission, and/or can be transmitted directly to a computer or communication device or can be transmitted indirectly via, such as, for example, but not limited to, an e-mail server, e-mail servers, or any other intermediate computer or communication device.

The present invention can also be utilized in order to process electronic commerce transactions regarding or involving the purchase and/or sale of any electronic interactive activities and/or any equipment, goods, or products, relating thereto to used in connection therewith.

The present invention can also be utilized to generate and/or transmit a reminder message, an alert message, or a notification message, to an individual, a patient, or a caregiver of same, containing information regarding the availability of an electronic interactive activity or electronic interactive activities, to generate and/or transmit a reminder message, an alert message, or a notification message, to an individual, a patient, or a caregiver of same, regarding a time or a scheduled time of a prescribed time when an individual or a patient is to engage in performing or participating in an electronic interactive activity or electronic interactive activities, or to generate and/or transmit a reminder message, an alert message, or a notification message, to a healthcare provider, any other provider, or a caregiver, regarding an individual's or a patient's engagement in or use of, and/or the individual's or the patient's failure to engage in or failure to use, an electronic interactive activity or electronic interactive activities pursuant to a prescribed treatment or treatment plan, and/or including any data, information, or results, of or obtained from the individual's or a patient's engagement in or use of an electronic interactive activity or electronic interactive activities.

The present invention can also be utilized in connection with social networks and any and/or all social networking support group members, and/or individuals or patients invited to participate in a social networking support group, can each be verified, by utilizing the data and/or information stored in any and/or all of the electronic healthcare records, electronic medical records, electronic dental records, electronic pharmacy records, and/or electronic behavioral health records which are utilized in, or in connection with, the present invention as having been diagnosed with the same or a similar illness, sickness or condition, or as having been prescribed the same or a similar treatment or procedure, or as being prescribed the same or similar treatment plan, so as to maintain compliance with all healthcare and/or other privacy laws, rules, and regulations and other concerns as well as ensuring the integrity of the social networking support group.

The present invention can also be utilized in a same, similar, or analogous manner, in order to process and/or to provide veterinary healthcare information and/or veterinary healthcare-related information for and regarding any kind or type of animal or animals or any type or kind of pet or pets.

The present invention can also be utilized as a clearinghouse for facilitating the offering, selling, buying, trading, and/or other commerce and/or transactions, involving healthcare and/or healthcare-related services, products and/or goods.

### BRIEF DESCRIPTION OF THE DRAWINGS

In the Drawings:

FIG. 1 illustrates a preferred embodiment of the present invention, in block diagram form;

FIG. 2 illustrates the central processing computer of FIG. 1, in block diagram form;

FIG. 3 illustrates the provider communication device of FIG. 1, in block diagram form;

FIG. 4 illustrates the insurer or payer communication device of FIG. 1, in block diagram form;

FIG. 5 illustrates the user or patient communication device of FIG. 1, in block diagram form;

FIG. 6 illustrates the intermediary communication device of FIG. 1, in block diagram form;

FIG. 7 illustrates an exemplary healthcare records cloud system computer of FIG. 1, in block diagram form;

FIG. 8 illustrates an exemplary electronic interactive content/media computer of FIG. 1, in block diagram form;

FIG. 9 illustrates the an exemplary insurance exchange computer of FIG. 1, in block diagram form;

FIG. 10 illustrates an exemplary social networking computer of FIG. 1, in block diagram form;

FIG. 11 illustrates an exemplary media computer of FIG. 1, in block diagram form;

FIGS. 12A and 12B illustrate a preferred embodiment method of using the present invention, in flow diagram form;

FIG. 13 illustrates still another preferred embodiment method of using the present invention, in flow diagram form; and

FIGS. 14A and 14B illustrate yet another preferred embodiment method of using the present invention, in flow diagram form.

### DESCRIPTION OF THE PREFERRED EMBODIMENT

The present invention is directed to an apparatus and a method for processing and/or for providing healthcare information and/or healthcare-related information with or using an electronic healthcare record and information regarding and/or obtained with or from electronic interactive activity, information, content, or media, and, in particular, to an apparatus and a method for processing and/or for providing healthcare information and/or healthcare-related information with or utilizing an electronic healthcare record and information regarding and/or obtained with or from electronic interactive activity, information, content, or media, and any data and/or information contained therein for a number and variety of applications, and/or in order to provide an improved and comprehensive electronic healthcare record and healthcare records keeping apparatus and method which can provide enhanced functionality and a variety of functions, features and applications all of which can provide for enhancements and improvements in healthcare.

Electronic interactive activities and electronic interactive content or media can be used in treating and/or in diagnosing healthcare any illnesses, sicknesses, injuries, conditions, syndromes, states of dysfunction, and/or any healthcare or healthcare-related problems or impairments. The apparatus and method the present invention integrates the use of electronic interactive activities and electronic interactive content or media with electronic healthcare records in order to facilitating the treatment and/or the diagnosis of a wide range of healthcare illnesses, sicknesses, injuries, conditions, syndromes, states of dysfunction, and/or any healthcare or healthcare-related problems or impairments.

The apparatus and method of the present invention can also be utilized in order to identify or select an electronic interactive activity or activities and/or electronic interactive content or media for an individual or a patient, provide a venue for making same available to the individual or the patient, provide a venue or medium by which a healthcare provider and monitor or observe, or review information regarding, an individual's or a patient's use of, or engagement in, electronic interactive content or media or electronic interactive activity or activities, and provides a system and methods for integrating the use of electronic interactive activities and electronic interactive content or media in serving the healthcare needs of individuals or patients of all ages.

The apparatus and method the present invention can be utilized in order to integrate the use of electronic interactive activities and electronic interactive content or media with electronic healthcare records in various applications, including, but not limited to, treatment planning, treatment monitoring, diagnostic procedures or processes, therapy activities or exercises, rehabilitation activities or exercises, training activities or exercises, or retraining activities or exercises, and/or in any healthcare activities or healthcare-related activities.

The apparatus and method of the present invention can be utilized in order to provide an electronic interactive activity or electronic interactive activities via an electronic healthcare record or an electronic healthcare file and can be utilized by individuals and patients of any and/or all ages or age groups in order to provide for the use of an electronic interactive activity or electronic interactive activities which can be utilized in performing diagnostic evaluations or analyses, diagnoses, treatments, treatment planning, therapy, rehabilitation, training, or re-training, for and/or regarding any illnesses, sicknesses, injuries, conditions, syndromes, states of dysfunction, and/or any healthcare or healthcare-related problems or impairments, including, but not limited to, developmental, cognitive, physical, mental, behavioral, motor, speech, hearing, seeing, learning, or any other, disorders, illnesses, sicknesses, injuries, conditions, syndromes, or states of dysfunction, Alzheimer's disease, Dementia, learning disorders, attention deficit disorders, learning disabilities, muscular coordination disorders, chronic pain conditions, developmental disorders, speech disorders, cognitive disorders, mental acuity disorders, spatial disorders, temporal disorders, psychological disorders, physiological disorders, post traumatic stress disorders, lazy eye, atrophies of any kind or type, conditions or disorders that affect spatial and/or temporal conditions, muscular coordination and/or any other type of coordination or activity, or any other healthcare or healthcare-related disorder, illness, sickness, injury, condition, syndrome, or state of dysfunction.

As used herein, the terms “electronic interactive activity” (also abbreviated as “EIA”), “electronic interactive activities”, and “electronic interactive content or media”, as these terms are used herein, refers to, and means, any type or kind of video game or video games, audio game or audio games, video and audio game or video and audio games, electronic or digital exercise or exercises, electronic or digital puzzle or puzzles or activity or activities, electronic or digital diagnostic activity or activities or tool or tools, electronic or digital treatment, treating planning, or therapy, activity or activities or tool or tools, electronic or digital mental health or behavioral health activity or activities or tool or tools, electronic or digital physical or physical therapy activity or activities or tool or tools, electronic or digital training activity or activities or tool or tools, electronic or digital rehabilitation activity or activities or tool or tools, electronic or digital mental health or behavioral health training or re-training activity or activities or tool or tools, electronic or digital physical training or re-training activity or activities or tool or tools, survey or surveys, questionnaire or questionnaires, or any other game or games, exercise or exercises, activity or activities, information gathering event or events or occurrence or occurrences, or any other content or media which can be utilized in connection with a computer, a computer system, a communication device, or any other electronic device, in order to obtain any data and/or information from an individual or patient, and/or which can be used in performing a diagnosis or a diagnostic evaluation or analysis, in identifying a treatment, a course of treatment, or a treatment plan, or in administering a therapy, rehabilitation activities, training activities, re-training activities, or which can be used in any other manner in delivering or providing healthcare services or healthcare-related services.

As used herein, the term “electronic healthcare record” or “EHR” means any electronic healthcare record, electronic medical record, electronic dental record, electronic pharmacy record, electronic behavioral health record, and/or any other record or record keeping system, software, hardware, or device, or any combination of same or group of same, which can contain healthcare information or healthcare-related information.

The apparatus and method of the present invention can be utilized in order to process information and/or to provide information regarding a diagnosis or possible diagnoses for or regarding an individual or a patient, to process and/or to provide diagnostic information based on a symptom or symptoms, an observation or observations, an examination finding or examination findings, any data and/or information obtained from a procedure performed on or with an individual or a patient or obtained from or during an examination, a test, a procedure, or an analysis, or any data and/or information obtained from or with any electronic interactive activity or electronic interactive activities described herein.

The apparatus and method of the present invention can also be utilized in order to process information and/or to provide information regarding a treatment, a prescribed treatment, or possible or available treatments, as well as any treatment plan or treatment plans associated with same, for an individual or a patient, which can include processing and/or providing data and/or information for or associated with identifying, prescribing, providing, administering, monitoring, recoding, and/or processing any data and/or information obtained from or with, any electronic interactive activity or electronic interactive activities which can be utilized in a treatment or an treatment plan for the individual or the patient.

The apparatus and method of the present invention can also be utilized to provide information regarding any electronic interactive activity or electronic interactive activities with or via an electronic healthcare record, provide access to an electronic interactive activity, provide or administer an electronic interactive activity or electronic interactive activities from or via an electronic healthcare record, provide for the monitoring, observing, and/or the recording of data and/or information, as well as any results obtained from and/or recorded, from an administration of an electronic interactive activity or electronic interactive activities, with, by, or using, an electronic healthcare record, or to utilize data and/or information obtained from an electronic interactive activity or electronic interactive activities, in performing follow-up diagnostic processing, treatment planning and/or treatment plan monitoring, and/or any healthcare information processing and/or healthcare-related processing for or regarding an individual or a patient.

The apparatus and method of the present invention can also be utilized to process data and/or information obtained with or from an electronic interactive activity or electronic interactive activities, provided from or via an electronic healthcare record in performing a diagnosis, in prescribing a treatment plan, in monitoring a treatment plan, in processing information for evaluating a treatment plan, in processing information for or regarding evaluating an individual's or a patient's progress or response to a treatment or a treatment plan, the effectiveness of the treatment or the treatment plan, or the progress or effectiveness of the treatment or treatment plan in meeting or achieving goals related to curing, healing, rehabilitating, training, or re-training, an individual or patient.

The apparatus and method of the present invention can also be utilized to provide and/or administer an electronic interactive activity, record and process data and/or information regarding an electronic interactive activity performed by an individual or a patient, allow for a healthcare provider or any other provider to monitor and/or observe the individual's or the patient's activities in performing an electronic interactive activity, allow for a healthcare provider or any other provider to monitor and/or observe the individual's or the patient's activities in performing an electronic interactive activity in real-time and/or otherwise, provide a recording of the individual's or the patient's activities in performing an electronic interactive activity to a healthcare provider or any other provider, and/or allow for a plurality or individual or patient's to engage in groups activities with or using an electronic interactive activity or electronic interactive activities.

The apparatus and method of the present invention can also be utilized to generate and/or transmit a reminder message, an alert message, or a notification message, to an individual, a patient, or a caregiver of same, containing information regarding the availability of an electronic interactive activity or electronic interactive activities, to generate and/or transmit a reminder message, an alert message, or a notification message, to an individual, a patient, or a caregiver of same, regarding a time or a scheduled time of a prescribed time when an individual or a patient is to engage in performing or participating in an electronic interactive activity or electronic interactive activities, or to generate and/or transmit a reminder message, an alert message, or a notification message, to a healthcare provider, any other provider, or a caregiver, regarding an individual's or a patient's engagement in or use of, and/or the individual's or the patient's failure to engage in or failure to use, an electronic interactive activity or electronic interactive activities pursuant to a prescribed treatment or treatment plan, and/or including any data, information, or results, of or obtained from the individual's or a patient's engagement in or use of an electronic interactive activity or electronic interactive activities.

The apparatus and method of the present invention can also be utilized to provide additional functions and/or functionality as described herein.

The apparatus and method of the present invention also facilitates the creation and management of a comprehensive electronic healthcare record system and a healthcare processing system, which can provide electronic interactive activities and/or electronic interactive content or media, which can also manage individual, patient, and/or client records, doctor and other provider records, healthcare insurance and/or payer records, and thereby provide an apparatus, system, and methods for providing a variety and a multitude of healthcare information processing applications, processes and services.

The apparatus and method of the present invention also provides for the integration of healthcare information and healthcare-related information which can be stored in or obtained from and among any number of different and separate electronic healthcare records and electronic healthcare records systems, which can be provided by any number of vendors or suppliers, and can facilitate a comprehensive electronic healthcare record or electronic healthcare record system.

The apparatus and method of the present invention can also provide for the integration of insurance exchanges and/or employee benefits exchanges and/or the respective computers or computer systems associated therewith with a healthcare information system, a healthcare-related information system, and/or an electronic healthcare record or electronic healthcare records.

The apparatus and method of the present invention can also provide for the integration of social networking computers and social networking web sites and their information and functionality with a healthcare information system, a healthcare-related information system, and/or an electronic healthcare record or electronic healthcare records.

The apparatus and method of the present invention can also provide for the integration of news, information, advertisements, and marketing information and/or materials, with or in connection with a healthcare information system, a healthcare-related information system, and/or an electronic medical record or electronic medical records.

The present invention facilitates improved healthcare quality, efficient information collection, processing and dissemination, efficient diagnosis and treatment, cost efficiency, cost containment, automated billing, notification and alert messaging, as well as many other benefits and advantages as will be described herein.

The apparatus and method of the present invention also facilitates the distribution and management of healthcare insurance, life insurance, disability insurance, as well as claims processing related thereto.

The present invention also provides an apparatus and a method for providing a comprehensive processing system which incorporates data and/or information from any combination and/or all of the participants in the healthcare field including, but not limited to, patients and those seeking healthcare, healthcare providers, doctors, including medical doctors, surgeons, physicians, dentists, orthodontists, periodontists, endodontists, oral surgeons, osteopaths, psychologists, optometrists, podiatrists, osteopaths, chiropractors, pharmacists, therapists, physical therapists, respiratory therapists, nurses, healthcare aids, nutritionists, and/or any other person, individual, and/or professional who can provide healthcare, healthcare-related, wellness and/or wellness-related services and/or products, insurance companies, healthcare insurance companies, disability insurance companies, property or casualty insurance companies, health maintenance organizations, healthcare providers, and any other payer and/or provider of healthcare services and/or products, healthcare claims processing centers, healthcare insurance brokers and/or agents, and/or any other third party and/or intermediary who or which acts on behalf of another and/or assists in to providing of healthcare and/or related services.

The apparatus and method of the present invention can also be utilized in order to maintain patient healthcare records or files private, to safeguard patient healthcare records or files, to provide notification to patients and other individuals when others have accessed, obtained, and/or made changes to their respective healthcare records or files, and/or to enable patients and individuals to restrict and/or limit access to their healthcare records or files.

As used herein, the terms “individual”, “patient”, “client”, “user” or the like, or their plural forms, refers to any person, individual, patient, entity, and/or client, who or which uses the present invention, and/or who seeks and/or who receives healthcare services, healthcare-related services, healthcare-related information, and/or any of the other services and/or products provided by the present invention.

As used herein, the term “caregiver” or the plural of same, refers to any parent, child, agent, attorney, representative, guardian, legal guardian, or any other person or entity who or which is responsible for caring for, looking after, or otherwise taking care of any healthcare needs of any individual or patient.

As used herein, the terms “doctor”, “healthcare provider”, “provider”, “therapist”, “healthcare information specialist”, etc., or their plural forms, refers to any medical doctor, including any and all of the various medical specialists and/or specialties, including, but not limited to internists, orthopedists, opthamalogists, cardiologists, hematologists, endocrinologists, oncologists, ears, nose and throat specialists, neurologists, urologists, gastrointerologists, dermatologists, pediatricians), medical specialist, surgeon, surgical specialists, including any and/or forms and/or types of surgeons), physician, dentist, psychiatrist, psychologist, optometrist, podiatrist, osteopath, chiropractor, pharmacist, therapist, physical therapist, respiratory therapist, nurse, healthcare aid, nutritionist, and/or any other person, individual and/or professional who can provide healthcare, healthcare-related, wellness, and/or wellness-related, services and/or products. The terms “healthcare provider” or “provider” can also refer to any provider or supplier of any of the herein-described electronic interactive content or media or any of the herein-described electronic interactive activities. The terms “healthcare provider” or “provider” can also refer to any person or individual who can prescribe, administer, monitor, or provide, an electronic interactive activity to any individual or patient.

As used herein, the terms “insurer”, “payer”, “insurance provider”, “heath insurance provider”, “life insurance provider”, “disability insurance provider”, etc., or their plural forms, refers to any insurance companies, healthcare insurance companies, disability insurance companies, property or casualty insurance companies, health maintenance organizations, healthcare providers, and any other payer and/or provider of healthcare services and/or products, who which provide and/or pay for healthcare and/or healthcare-related benefits, services, and/or products, and/or who or which provide respective health insurance, life insurance and/or disability insurance benefits, services and/or products.

As used herein, the terms “broker”, “agent”, “billing service”, “collection agent”, “manager”, intermediary”, “assistant”, etc., or their plural forms, refer to any broker, insurance broker, agent, insurance agent, intermediary, third party, billing service provider, collection agent, claim processing agent, and/or any other person, individual, and/or entity, which acts on behalf of, or for, any of the individuals, patients, doctors, healthcare providers, insurers, payers, etc., described herein.

Applicant hereby incorporates by reference herein the subject matter and teachings of U.S. Provisional Patent Application Ser. No. 61/914,290, filed Dec. 10, 2013, and entitled “APPARATUS AND METHOD FOR PROCESSING AND/OR PROVIDING HEALTHCARE INFORMATION AND/OR HEALTHCARE-RELATED INFORMATION WITH OR USING AN ELECTRONIC HEALTHCARE RECORD AND INFORMATION REGARDING AND/OR OBTAINED WITH OR FROM ELECTRONIC INTERACTIVE ACTIVITY, INFORMATION, CONTENT, OR MEDIA”, the subject matter and teachings of which are hereby incorporated by reference herein in their entirety.

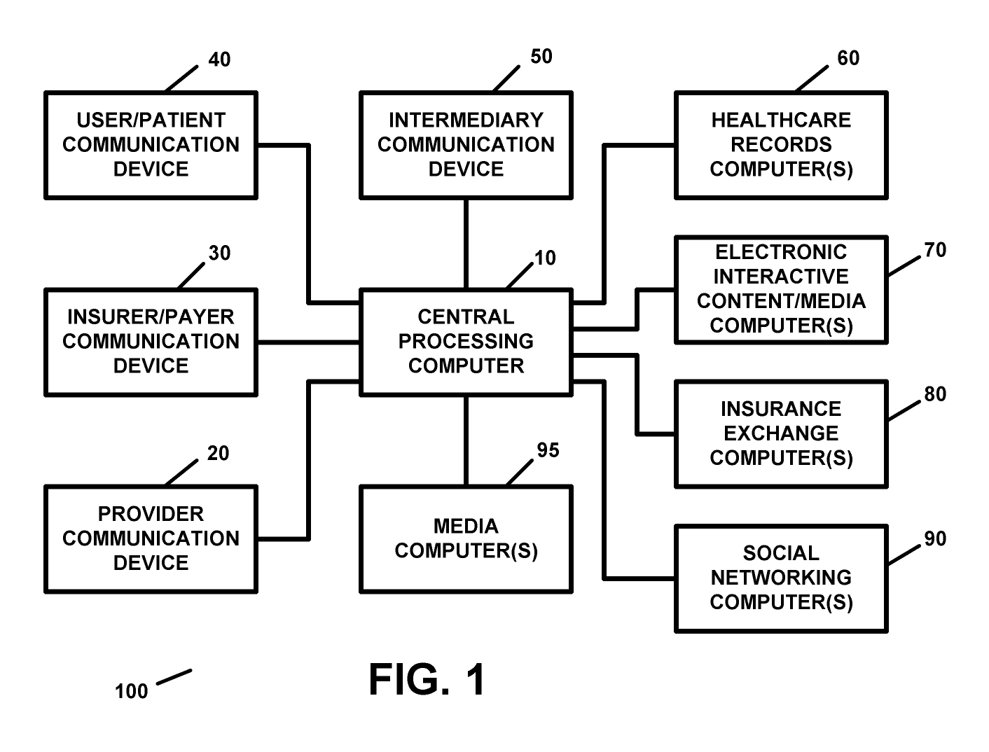

FIG. 1 illustrates the apparatus of the present invention, in block diagram form. The apparatus of the present invention is denoted generally by the reference numeral 100. In the preferred embodiment, the apparatus 100 of the present invention includes a central processing computer or central processing computer system 10 (hereinafter referred to as the “central processing computer 10”). In the preferred embodiment the central processing computer 10 can be a network or server computer.

In the preferred embodiment, the central processing computer 10 can provide control over the apparatus 100 and can perform any of the various processing services and/or functions described herein. The central processing computer 10 may be a single computer or system of computers and/or may be include a plurality of computers or computer systems which are utilized in conjunction with one another. The central processing computer 10, in the preferred embodiment can provides services for any of the other computers and/or computer systems described herein as being associated with any of the individuals, patients, healthcare providers, insurers, payers, brokers, agents, and/or intermediaries, described herein.

The apparatus 100 also includes a healthcare provider communication device or computer 20 (hereinafter referred to as “provider communication device 20” or “provider computer 20”) which is associated with a healthcare provider such as a healthcare professional, a hospital, a clinic, and/or any other provider of services described herein. A provider computer 20 can also be associated with, or can be used by, any research institution, research facility, teaching institution, teaching hospital, college, university, school, or other institution or entity which may perform research, or provide research information or any related information regarding studies, findings, or developments, in the healthcare field or in healthcare-related fields. A provider computer 20 can also be associated with, or can be used by, any public health department, public health agency, public health facility, or other public health entity, on any one or more of a national level, a country level, a state level, a provincial level, a county level, a city level, a municipal level, or any other level, which is entrusted to provide for the public health and/or to perform services or oversight regarding public health. A provider computer 20 can also be associated with, or can be used by, any immunization registry or immunization registries. A provider computer 20 can also be associated with, or can be used by, any source or provider of educational or instructional information, or information regarding instructions or procedures, or links or hyperlinks to same.

Any number or amount of healthcare provider computers 20 can be utilized in conjunction with a healthcare provider and/or group of providers. The healthcare provider communication device(s) 20 can communicate with, and operate in conjunction with, the central processing computer 10 and/or any of the other computers and/or computer systems associated with any of the other individuals and/or entities which utilize and/or operate in conjunction with the present invention.

The apparatus 100 can also include a healthcare insurer or payer communication device or computer 30 (hereinafter referred to as “payer communication device 30” or “payer computer 30”) which is associated with a healthcare payer such as a healthcare insurer, insurance company, health maintenance organization, a clinic, and/or any other payer of healthcare services and products described herein. Any number or amount of healthcare payer computers 30 can be utilized in conjunction with a healthcare payer and/or group of payers. The healthcare payer communication device(s) 30 can communicate with, and operate in conjunction with, central processing computer 10 and/or any of the other computers and/or computer systems associated with any of the other individuals and/or entities which utilize and/or operate in conjunction with the present invention.

The apparatus 100 can also include a user, patient, or individual, communication device or computer 40 (which may hereinafter be referred to either as “user communication device 40”, “user or patient communication device 40”, “user computer 40”, “patient computer 40”, or “user/user or patient communication device 40”) which is associated with any user of the apparatus 100 or any individual or healthcare patient or client who seeks or who is provided with healthcare and/or related services, products and/or related information. The user or patient communication device 40 can also be utilized by any individual, party, or entity, who or which may merely utilize the present invention in order to care or another individual or to obtain information of interest.

A user or user or patient communication device 40 may also be located at public places or locations, such as at kiosks or other publicly available computer or communication devices. Any number or amount of user or patient computers 40 can be utilized in conjunction with a user, patient, group of users, and/or group of patients. The user or patient communication device 40 can communicate with, and operate in conjunction with, the central processing computer 10 and/or any of the other computers and/or computer systems associated with any of the other individuals and/or entities which utilize and/or operate in conjunction with the present invention. The user or patient communication device 40 can also be utilized by any other individual or entity desiring to utilize and/or to obtain information from the apparatus 100 and/or from any other the

The apparatus 100 can also include an intermediary communication device or computer 50 (hereinafter referred to as “intermediary communication device 50” or “intermediary computer 50”) which is associated with an intermediary, a broker, an agent, and/or any other individual and/or entity, that can utilize the present invention in order to act for and/or on behalf of any other individual, party, or entity, described herein. In a preferred embodiment, the intermediary communication device 50 can also be utilized by any provider of any of the herein-described electronic interactive content or media or of any of the herein-described electronic interactive activities. Any number or amount of intermediary computers 50 can be utilized in conjunction with an intermediary and/or group of intermediaries. The intermediary computer(s) 50 can communicate with, and operate in conjunction with, the central processing computer 10 and any of the other computers and/or computer systems associated with any of the other individuals and/or entities which utilize and/or operate in conjunction with the present invention.

In the preferred embodiment, any of the provider communication device(s) 20, the payer computer(s) 30, the patient computer(s) 40, and/or the intermediary computer(s) 50, can be any computer or communication device, including, but not limited to, a personal computer, a home computer, a server computer, a network computer, a hand-held computer, a palmtop computer, a laptop computer, a personal communication device, a cellular telephone, a wireless telephone, a mobile telephone, a digital television, an interactive television, a digital television, a personal digital assistant, a telephone, a digital telephone, a television, an interactive television, a beeper, a pager, and/or a watch.

Each of the central processing computer(s) 10, the provider communication device(s) 20, the payer communication device(s) 30, the patient computer(s) 40, and/or the intermediary computer(s) 50, can transmit information to, as well as receive information from, any of the computers 10, 20, 30, 40, and 50, described herein. In this regard, each of the computers 10, 20, 30, 40, and 50, can communicate with, process information from, and/or share data and/or information with, each other and/or any other computer or computers 10, 20, 30, 40, and 50, described herein and/or utilized in conjunction with the present invention. In this manner, data and/or information transfer between any of the computers 10, 20, 30, 40, and 50, can communicate with any other computer or computers 10, 20, 30, 40, and 50, in a bi-directional manner.

The central processing computer(s) 10, the provider communication device(s) 20, the payer computers(s) 30, the patient computer(s) 40, and the intermediary computer(s) 50, can communicate with one another, and/or be linked to one another, over a communication network, a telecommunication network, a telephone network, a line-connected network, and/or a wireless communication network. Each of the computers 10, 20, 30, 40, and 50, can be linked with any other computer or computers directly or indirectly directly or indirectly with one another so as to facilitate a direct or indirect bi-directional communication said respective computers. Communications between each of the computers 10, 20, 30, 40, or 50 can also involve an e-mail server or e-mail servers in those instances when e-mails are described as being used to transmit or send any of the information, signals, messages, reports, notification messages, or any other communications, described herein, by or between any of the computers 10, 20, 30, 40, or 50, or when any of the information, signals, messages, reports, notification messages, or any other communications, described herein, are transmitted by and/or between any of the parties described herein and/or by or between any of the computers 10, 20, 30, 40, or 50, or any other computers or communication devices, computer systems, communication network equipment, server computers, etc., or any other devices used or needed in order to facilitate communications or the transmission of any of the herein-described information, signals, messages, reports, notification messages, or any other communications.

The apparatus 100 can also include a healthcare records computer 60 or computers (hereinafter referred to as “healthcare records computer 60” or “healthcare records computer(s) 60) which can be or can include a computer or computer system, or any number of computers or computer systems, or a cloud computer system or cloud system. The healthcare records computer can serve to store and house an electronic healthcare record or files or any number of electronic healthcare records or files. In the preferred embodiment, the healthcare records computer 60 can be associated with any provider, insurer, payer, intermediary, insurance exchange, or any user, individual, patient, organization, or entity, who or which utilizes the apparatus 100 and method of the present invention.

Each healthcare records computer 60 can be utilized to store an electronic healthcare record or file or any number of electronic healthcare records or files which can be accessed by the central processing computer 10, by any provider communication device 20, by any insurer or payer communication device 30, by any user or patient communication device 40, by any intermediary communication device 50, or by any other computer, communication device or other device described herein as being utilized in connection with the apparatus 100 and method of the present invention. The healthcare records computer 60 can also be utilized to facilitate cloud storage of any electronic healthcare record(s) or file(s).

The apparatus 100 can also include an electronic interactive content/media computer 70 or computers (hereinafter referred to as “electronic interactive content/media computer 70” or “electronic interactive content/media computer(s) 70) which can be or can include a computer or computer system, or any number of computers or computer systems, or a cloud computer system or cloud system. The electronic interactive content/media computer 70 can serve to store and house any of the herein-described electronic interactive content or media described herein which can be provided by or which can be supplied by any provider or supplier of same or any number, types, or kinds, of electronic interactive content or media which can be provided by or supplied by any provider or supplier of same or any number of providers or suppliers of same. In the preferred embodiment, the electronic interactive content/media computer 70 can be associated with any provider or supplier, or vendor, of any electronic interactive content or media or any electronic interactive activity who or which utilizes the apparatus 100 and method of the present invention.

Each electronic interactive content/media computer 70 can be utilized to store electronic interactive content or media or any number, types, or kinds, of electronic interactive content or media or electronic interactive activities which can be accessed by the central processing computer 10, by any provider communication device 20, by any insurer or payer communication device 30, by any user or patient communication device 40, by any intermediary communication device 50, or by any other computer, communication device or other device described herein as being utilized in connection with the apparatus 100 and method of the present invention. The electronic interactive content/media computer 70 can also be utilized to facilitate cloud storage of any electronic interactive content or media or of any electronic interactive activities.

The apparatus 100 can also include an insurance exchange computer 80, or any number of insurance exchange computers 80 which can be utilized to process and store information regarding the selling of healthcare insurance, disability insurance, and life insurance, policies, products, and/or services, to any of the herein-described users, individuals, patients, or entities, who or which utilize the apparatus 100 and method of the present invention. The insurance exchange computer 80 can be utilized to advertise, provide information regarding, sell, and/or maintain records regarding, and process any other information regarding, group insurance as well as individual or family insurance policies, products, or services. The insurance exchange computer 80 can also be utilized to sell automobile, homeowners, business, and/or liability insurance policies, products, or services.

The apparatus 100 can also include a social networking computer 90. The social networking computer 90 can be linked with, and utilized in connection with, the apparatus 100 so as to allow and/or facilitate integrating the apparatus 100 of the present invention with social networks, social networking, and social media. In a preferred embodiment, for example, the social networking computer 90 can be associated with a social networking company, a social networking website, or social networking entity, website, group, organization, or association. The social networking computer 90 can be associated with any one or any number of social networking companies, social networking websites, or social networking entities, websites, groups, organizations, or associations. The social networking computer 90 can also provide links to any computers associated with any one or any number of social networking companies, social networking websites, or social networking entities, websites, groups, organizations, or associations. In the preferred embodiment, the social networking computer 90 can perform any and all of the functions performed by any social networking company, a social networking website, or social networking entity, website, group, organization, or association. In a preferred embodiment, any number of social networking computers 90 can be utilized in connection with the apparatus 100 of the present invention.

The apparatus 100 can also include a media computer 95. In the preferred embodiment, the media computer 95 can provide, and be a source of, news information, current events information, healthcare information, healthcare or healthcare-related news, current events, advertisements, and/or marketing information or materials, which can be disseminated or distributed via the apparatus 100 of the present invention, In a preferred embodiment, any number of media computers 95, with each being dedicated to providing any number, types, or kinds of, news information, current events information, healthcare information, healthcare or healthcare-related news, current events, advertisements, and/or marketing information or materials, can be utilized in connection with the apparatus 100.

In a preferred embodiment, each of the central processing computer(s) 10, the provider communications devices 20, the payer communication devices 30, the user or patient communication devices 40, the intermediary communication devices 50, the healthcare records computers 60, the electronic interactive content/media computers 70, the insurance exchange computers 80, the social networking computers 90, and the media computers 95 can communication in a bi-directional manner with, and/or can send and/or receive signals, messages, reports, notification messages, alerts, or any other communications or electronic communication transmissions, to, from and/or between, any other, or any number of, other central processing computer(s) 10, if utilized, provider communications devices 20, payer communication devices 30, user or patient communication devices 40, intermediary communication devices 50, healthcare records computers 60, electronic interactive content/media computers 70, insurance exchange computers 80, social networking computers 90, and/or the media computers 95.

In a preferred, each of the central processing computer(s) 10, the provider communications devices 20, the payer communication devices 30, the user or patient communication devices 40, the intermediary communication devices 50, the healthcare records computers 60, the electronic interactive content/media computers 70, the insurance exchange computers 80, the social networking computers 90, and the media computers 95 can be linked to or with any other central processing computer(s) 10, if utilized, provider communications devices 20, payer communication devices 30, user or patient communication devices 40, intermediary communication devices 50, healthcare records computers 60, electronic interactive content/media computers 70, insurance exchange computers 80, social networking computers 90, and/or the media computers 95, via a wired link or line or a wireless link.

In a preferred embodiment, each of the provider communications devices 20, payer communication devices 30, user or patient communication devices 40, intermediary communication devices 50, healthcare records computers 60, electronic interactive content/media computers 70, insurance exchange computers 80, social networking computers 90, and/or the media computers 95, can be connected with or linked with the central processing computer 10 as shown in FIG. 1.

In a preferred embodiment, any and/or all of the signals, messages, reports, notification messages, or any other communications, described herein as being transmitted from one device, computer, or communication device, to another, can be, or can be included in, or be attached to, an e-mail message, an instant messaging message, an electronic transmission, or an electronic data transmission or electronic data interchange, or can be transmitted via any other data or information transmission, and can be transmitted via or using any appropriate or necessary computer(s) or device(s).

In the preferred embodiment, the present invention is utilized on, and/or over, the Internet and/or the World Wide Web. The present invention, in the preferred embodiment, can also utilize wireless Internet and/or World Wide Web services, equipment and/or devices. The central processing computer(s) 10, in the preferred embodiment, has a web site or web sites associated therewith. Each of the other computers or communication devices described herein can also have a web site or web sites associated with same.

Although the Internet and/or the World Wide Web is a preferred communication system and/or medium utilized, the present invention, in all of the embodiments described herein, can also be utilized with any appropriate communication network or system including, but not limited to, a communication network or system, a telecommunication network or system, a telephone communication network or system, a cellular communication network or system, a wireless communication network or system, a line or wired communication network or system, a wireless Internet network or system, a wireless World Wide Web network or system, a digital communication network or system, a personal communication network or system, a personal communication services (PCS) network or system, a satellite communication network or system, a broad band communication network or system, a low earth orbiting (LEO) satellite network or system, a public switched telephone network or system, a telephone communication network or system, a radio communication network or system, a cable television network or system, and/or any other communication network or system, and/or any combination of the above communication networks or systems.

In the preferred embodiment, each of the central processing computer(s) 10, the provider communication device(s) 20, the payer communication device(s) 30, the patient computer(s) 40, the intermediary computer(s) 50, the healthcare records computer(s) 60, the electronic interactive content/media computer(s) 70, the insurance exchange computer(s) 80, the social networking computer(s) 90, and the media computer(s) 95, can transmit data and/or information using TCP/IP, as well as any other Internet and/or World Wide Web, and/or communication, protocols.

The apparatus 100 of the present invention can utilize electronic commerce technologies and security methods, techniques and technologies, in any and/or all of the instances of data and/or information processing, and/or data and/or information transmission described herein.

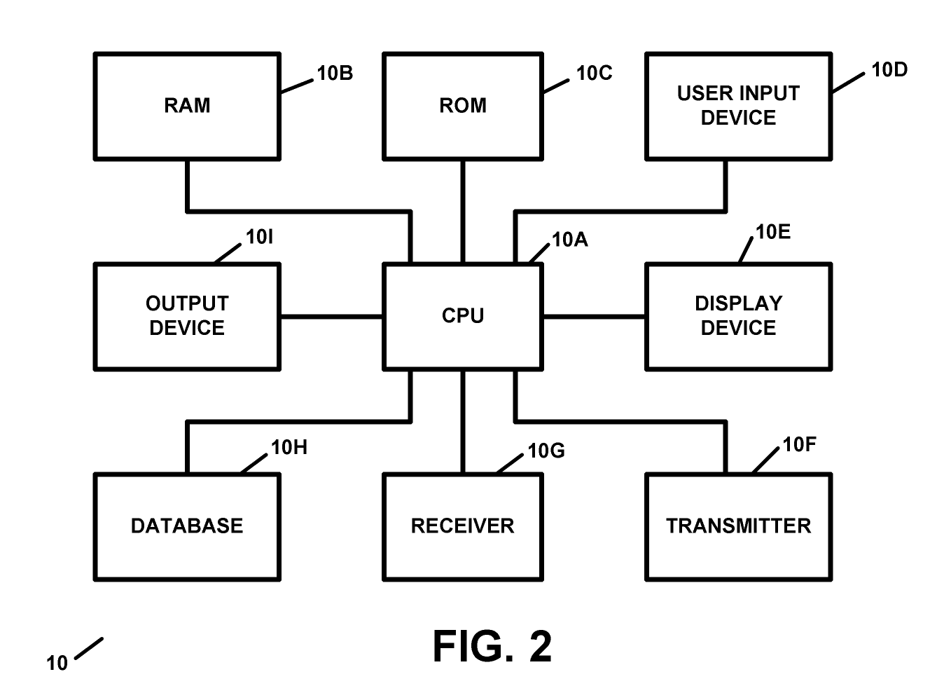

FIG. 2 illustrates the central processing computer 10, in block diagram form. The central processing computer 10, in the preferred embodiment, is a network computer or computer system, or any other communication device which can provide the functionality of, and which can be utilized as a central processing computer such as an Internet server computer and/or a web site server computer. In the preferred embodiment, the central processing computer 10 includes a central processing unit or CPU 10A, which in the preferred embodiment, is a microprocessor. The CPU 10A may also be a microcomputer, a minicomputer, a macro-computer, and/or a mainframe computer, depending upon the application.

The central processing computer 10 also includes a random access memory device(s) 10B (RAM) and a read only memory device(s) 10C (ROM), each of which is connected to the CPU 10A, a user input device 10D, for entering data and/or commands into the central processing computer 10, which includes any one or more of a keyboard, a scanner, a user pointing device, such as, for example, a mouse, a touch pad, and/or an audio input device and/or a video input device, a microphone or audio recording device, a camera or a video recording device, and/or any device, electronic and/or otherwise which can be utilized for inputting and/or entering healthcare data and/or information, for example pulse rate monitors, blood pressure monitors, electrocardiograms, blood-sugars monitors, etc., if desired, which input device(s) are also connected to the CPU 10A. The central processing computer 10 also includes a display device 10E for displaying data and/or information to a user or operator.

The central processing computer 10 also includes a transmitter(s) 10F, for transmitting signals and/or data and/or information to any one or more of the central processing computer(s) 10, the provider communication device(s) 20, the payer computers(s) 30, the patient computer(s) 40, and the intermediary computer(s) 50, the healthcare records computer(s) 60, the electronic interactive content/media computer(s) 70, the insurance exchange computer(s) 80, the social networking computer(s) 90, and the media computer(s) 95, or any other individual computer(s), which may be utilized in conjunction with the present invention. The central processing computer 10 also includes a receiver 10G, for receiving signals and/or data and/or information from any one or more of the central processing computer(s) 10, the provider communication device(s) 20, the payer computers(s) 30, the patient computer(s) 40, and the intermediary computer(s) 50, the healthcare records computer(s) 60, the electronic interactive content/media computer(s) 70, the insurance exchange computer(s) 80, the social networking computer(s) 90, and the media computer(s) 95, which may be utilized in conjunction with the present invention.

The central processing computer 10 also includes a database(s) 10H which contains data and/or information pertaining to the patients, providers, payers, and intermediaries who or which are serviced by the present invention and/or who or which utilize the present invention.

The database 10H can contain any one or any number of electronic healthcare records (EHRs), which electronic healthcare records definition includes electronic healthcare records or files, electronic medical records or files (EMRs), electronic dental records or files (EDRs), electronic pharmacy records or files (EPRs), electronic behavioral health records or files (EBHRs), as well as any personal health records or files (PHRs). The database 10H can also contain any links or hyperlinks to any of the above electronic healthcare records or files, electronic medical records or files, electronic dental records or files, electronic pharmacy records or files, electronic behavioral health records or files, and/or personal health records or files, which may be located or stored on any computer or computers external from the central processing computer 10, for any number of users, individuals, or patients.

Each of the herein-described electronic healthcare records, electronic medical records, electronic dental records, electronic pharmacy records, and/or electronic behavioral health records can also contain a personal health record portion. The apparatus 100 and method of the present invention can store a personal health record for each individual or patient. The personal health record can be stored in each individual's or patient's respective electronic healthcare record(s), electronic medical record(s), electronic dental record(s), electronic pharmacy record(s), and/or electronic behavioral health record(s), as well as in any user or patient communication device 40. The personal healthcare record can be updated as described herein or in any other appropriate manner.

The database 10H can also contain a look-up table or look-up tables and/or data and/or information relating thereto, for identifying and locating a respective electronic healthcare record(s), an electronic medical record, an electronic dental records, an electronic pharmacy record, an electronic behavioral health record, or a personal health records, for or associated with any user, individual, patient, or person who may utilize the apparatus 100 of the present invention. In this regard, the database 10H can store any healthcare data and/or information or healthcare-related data and/or information for any user, individual, patient, or person who may utilize the apparatus 100 of the present invention, whether that information is stored in the central processing computer 10, the database 10H, or any provider communication device(s) 20, payer communication device(s) 30, user or patient communication device(s) 40, intermediary communication device(s) 50, healthcare records computer(s) 60, electronic interactive content/media computer(s) 70, insurance exchange computer(s) 80, social networking computer(s) 90, and/or media computer(s) 95, regardless of where these computers or communications devices are located and regardless of what data and/or information is stored on same.

In the preferred embodiment, the database 10H contain data and/or information for locating, retrieving, storing, or providing a comprehensive healthcare record of healthcare file for any individual, user, patient, or person. The database 10H can also contain any necessary data or information for locating, identifying, retrieving, storing, any information from any of the various types of kinds of electronic healthcare records or files regarding of the type, kind, and/or vendor or supplier of same, regardless of where these electronic healthcare records or files are located or stored. In a preferred embodiment, the database 10H can also contain an index or other information for identifying data fields in the respective databases of the various respective electronic healthcare records for enabling the central processing computer 10 to identify data and/or information in each of the respective electronic healthcare records or files.

The database 10H can also contain any data and/or information for allowing any herein-described user, individual, patient, person, provider, payer, intermediary, or entity, to access or obtain any of the herein-described data and/or information, wherever such data and/or information is located or stored, as well as to perform any function described herein as being capable of being performed by the apparatus 100 of the present invention.

The database 10H can also contain any data and/or information for integrating, linking to or with, and/or accessing data and/or information stored or located in, any number, type, or kind, of any of the herein-described electronic healthcare records or files, electronic medical records or files, electronic dental records or files, electronic pharmacy records or files, electronic behavioral health records or files, as well as any personal health records or files.

The database 10H can contain any of the herein-described data and/or information for any number of individuals, users, patients, or persons. The database 10H can also contain data and/or information regarding a name, relationship, and authorization for any parent, relative, legal guardian, caregiver, healthcare proxy holder, or any person, individual, or entity, who or which may be responsible for caring for, accessing, updating, or maintaining healthcare records or files for, making decisions for, or making healthcare-related decisions for, taking actions for, providing care for, or performing any task or activity for, any other individual(s), user(s), patient(s), or person(s). For example, the database 10H can, for a child, contain information regarding the child's parent or legal guardian, and for the database 10H, for an elderly person of any other person, contain information regarding the person's child, caregiver, healthcare proxy holder, or any other authorized person or entity, or for individuals who belong to a school or club's athletic program, or a sports team, a club, a social or other organization, the database 10H can contain information regarding a coach, team doctor, trainer or other authorized individual.

The database 10H can also contain and/or include any of the herein-described electronic interactive content or media and/or any of the herein-described electronic interactive activities described herein as being provided by or via the apparatus 100 and method of the present invention. The database 10H can also contain and/or include any link or links, or hyperlink or hyperlinks, to any of the herein-described electronic interactive content or media and/or to any of the herein-described electronic interactive activities described herein as being provided by or via the apparatus 100 and method of the present invention.

The a database 10H can also contain and/or include, for each individual or patient who utilizes the apparatus 100 and method of the present invention or for whom the apparatus 100 and method of the present invention can be utilized, any data and/or information regarding any electronic interactive content or media or any electronic interactive activity or activities which are to be, or which have been, provided to, administered to, or utilized by, the individual or patient, a description of same, a description of any illness, sickness, injury, condition, syndrome, state of dysfunction, and/or any healthcare or healthcare-related problem or impairment, for which the respective electronic interactive content or media or any electronic interactive activity or activities may be useful in treating or diagnosing, the provider, supplier, or vendor of same, the healthcare provider who has prescribed same, any diagnostic report(s), treatment report(s), or any evaluation report(s), or any data and/or information contained therein, or any other healthcare or healthcare-related data and/or information, which identifies or includes information regarding, or which suggests or calls for, a need or a use of same for the individual or the patient, the date and time of the respective diagnostic report(s), treatment report(s), or any evaluation report(s), or any data and/or information contained therein, or any other healthcare or healthcare-related data and/or information, the healthcare provider prescribing same, and/or any other data and/or information regarding any data and/or information regarding any electronic interactive content or media or any electronic interactive activity or activities which are to be, or which have been, provided to, administered to, or utilized by, the individual or patient.

The database 10 can also contain and/or include data and/or information regarding any provider, supplier, or vendor of, as well as any date of or any date or any dates of any updates to any, and/or any information regarding any reviews of or regarding, any electronic interactive content or media or any electronic interactive activity or activities utilized in connection with the apparatus 100 and method of the present invention.

The database 10 can also contain and/or include data and/or information, for each individual or patient, any data and/or information regarding any prescribed and/or or scheduled dates and/or times when the individual or the patient is to access, be provided with, utilize, or engage or participate in using, and electronic interactive content or media or any electronic interactive activity or activities utilized in connection with the apparatus 100 and method of the present invention, the prescribing healthcare provider, the respective diagnostic report(s), treatment report(s), or any evaluation report(s), or any data and/or information contained therein, or any other healthcare or healthcare-related data and/or information, which calls for, prescribes, or contains information regarding any need or suggestion for, or which calls for the use of, a respective electronic interactive content or media or any electronic interactive activity or activities.

The database 10H can also contain and/or include, for each individual or patient, any data and/or information regarding the individual's or the patient's accessing of, playing or, use of, engagement in, or participation in, any electronic interactive content or media or any electronic interactive activity or activities, including, but not limited to, the date and time of such accessing of, playing of, use of, engagement in, or participation in, same, whether or not the individual or the patient accessed same, played same, used same, engaged in same, or participated in same, alone or with other individuals or patients, and in the case where other individuals or patients were involved in the accessing of, the playing of, the use of, the engagement in, or the participation in, same, the names and/or identities of those other individuals or patients.

The database 10H can also contain and/or include, for each individual's or patient's accessing of, playing or, use of, engagement in, or participation in, any of the electronic interactive content or media or any electronic interactive activity or activities provided by the apparatus 100 and method of the present invention, any data and/or information regarding any information provided by or input or entered by the individual or the patient, any data and/or information regarding the individual's or the patient's actions and/or performance, and/or any data and/or information regarding any results, or any analysis or evaluation of any results of or regarding the individual's or the patient's accessing of, playing or, use of, engagement in, or participation in, the electronic interactive content or media or any electronic interactive activity or activities.

The database 10H can also contain and/or include, for each individual's or patient's accessing of, playing or, use of, engagement in, or participation in, any of the electronic interactive content or media or any electronic interactive activity or activities provided by the apparatus 100 and method of the present invention, any data and/or information regarding any monitoring, observation, analysis, or evaluation, performed by or made by a healthcare provider or other provider, and/or any data and/or information regarding the date and/or times of such monitoring, observation, analysis, or evaluation, any notes, data and/or information regarding same, and/or the name or identify of that healthcare provider or other provider performing or providing same.

The database 10H can also contain and/or include any data and/or information needed for correlating any of the herein-described electronic interactive activities to any diagnoses and/or treatments, and/or for correlating any of the herein-described electronic interactive activities with any symptoms, diseases, conditions, illnesses, syndromes, sicknesses, and/or states of dysfunction. The database 10H can also contain and/or include a data and/or information contained in a look-up table-type arrangement and/or any other type of data and/or information association or correlative relationship arrangement which can relate or correlate the use of any electronic interactive activity as a diagnostic indicator or tool for, as a treatment activity or tool for, as a treatment planning activity indicator or tool for, as a training indicator or tool, as a rehabilitation indicator or tool, or as a re-training indicator or tool for, any symptom, disease, condition, illness, syndrome, sickness, and/or state of dysfunction. In this manner, the database 10H can contain any data and/or information needed of desired for identifying and/or selecting, or prescribing, any electronic interactive activity or any electronic interactive activities in and/or for any processing of and/or any generation of any information, reports, or messages, regarding any healthcare information and/or healthcare-related information described herein as be processed by or generated by the apparatus 100 and method of the present invention, as described herein.

The database 10H can contain any and/or all of the information needed and/or required in order to perform any and/or all of the functions, services and/or operations described herein as being performed by the central processing computer 10 or the apparatus 100 of the present invention. In this regard the database 10H can contain data and/or information regarding patient name, patient identification information, patient social security number or other identification information, date of birth, sex, gender, race, nationality, ethnicity, spoken language, preferred language, other languages, religion or religious affiliation, next-of-kin, relatives, emergency contact person, doctors or providers, therapists, nutritionists, insurance or payer information, group insurance information, group health insurance information, life insurance information, disability insurance information, patient address, phone number, e-mail and/or other contact information, medical history, psychological history, dental history, family history, family medical, psychological, and/or dental history, past and/or current or active symptoms, past and/or current or active diagnoses, past and/or current or active treatments or treatment plans, care management plan or plans, allergies, active or current medications or prescription drugs, active or current medication allergies, past, active, or current allergies, smoking status, insurance coverage, insurance co-payment and/or deductible information, co-payment information regarding any payer or insurer insurance policy or payment plan, deductible information regarding any payer or insurer insurance policy or payment plan, benefit eligibility information, insurance information, insurance claim procedures, insurance claim forms, doctor or provider appointment schedules, past treatments, past diagnosis, symptoms, insurance claim forms, employer information, lifestyle information, treatment plans, treatment progress, broker/agent/intermediary information, education information, age, sex, marital status, employee benefits information, types or services and/or treatments needed, and any other data and/or information regarding the patient which would be needed and/or desired in order to perform any and/or all of the functions, services and/or operations described herein.

The database 10H can also contain provider office visit summaries, hospital discharge summaries, hospital discharge instructions, and/or any examination, treatment, testing, or procedure, summary, and any instructions, treatment plan or instructions, care management plan or instructions, or any other information which can be provided to any individual, patient, provider, payer, insurer, third party or intermediary, or caregiver, described herein as using the apparatus 100 and method of the present invention, or described herein as being able to receive services, provide services, or otherwise use or be serviced by, the apparatus 100 and method of the present invention.

In the case of deceased patients or individuals, the database 10H can contain information regarding date of death, place of death, preliminary cause of death, and/or final determination of cause of death. The database 10H can also contain information regarding any other information regarding a deceased patient or individual and can also contain information regarding final resting place.

The database 10H can also contain, for each patient or individual, information regarding dates of appointments or services with or from healthcare providers or other providers, dates of laboratory tests, dates of treatments, operations, or procedures, dates of doctor visits, therapy sessions, vital signs, healthcare chart changes, or information regarding height, weight, blood pressure, heart rate, pulse rate, body-mass index, growth charts, and/or any other related or pertinent data or information.

The database(s) 10H can also contain healthcare and/or medical video, image, and/or audio, and/or text, data and/or information, and/or surgical video, image, and/or audio, and/or text, data and/or information, and/or dental video, image, and/or audio, and/or text, data and/or information, such as, for example, x-rays, Magnetic Resonant Images (MM), CAT scans, digital x-ray files, digital Magnetic Resonant Imaging (MRI) files, digital CAT scan files, and/or any other video, imaging, and/or audio, healthcare data and/or information which can be utilized by healthcare providers, payers, intermediaries, patients, and/or other users of the present invention. In this manner, the present invention can facilitate the availability of any of the above-described video, image, and/or audio, data and/or information in a network environment. For example, a medical specialist can have access to, and/or review, an MRI or a CAT scan for a patient, from any location and at any time.

The database 10H can also contain data and/or information regarding providers including provider name, provider social security number or identification number, type of professional or service provider, address, phone number, fax number, e-mail and/or other contact information, experience, specialties, insurances accepted, schedule of charges, financial account identification information, resume information, education, work experience, claim forms, appointment schedules, procedures performed, and/or any other data and/or information concerning the providers for providing any and/or all of the functions, services, and/or operations described herein as being performed by the present invention.

The database 10H can also contain data and/or information regarding all possible fields of medicine, surgery, psychiatry, psychology, dentistry, oral surgery, optometry, podiatry, physical therapy, respiratory therapy, hypnosis, osteopathy, nutrition, wellness, and/or any other possible healthcare fields and/or subject matter which can possibly by utilized in the processing and/or operation of the present invention.

The database 10H can also contain information on illnesses, symptoms, diseases and/or sicknesses, theories, scientific theories, research data and/or information, diagnosis information, treatment information, treatment plans, treatment processes, treatment progresses, treatment interactions, side effects, expected treatment results, treatment providers, treatment durations, treatment costs, pre-treatment information, post-treatment information, treatment monitoring information, statistical information regarding diagnoses, treatments, treatment success rates, treatment failure rates, treatment centers, therapy plans, therapy success rates, therapy failure rates, treatment procedures, medications treatments, non-medication treatments, healthcare institutions, treatment evaluating criteria, treatment mistakes and/or mishaps, indicators of mistakes and/or mishaps, corrective actions, links to providers, links to treatment centers or institutions, reimbursement rates, nutrition information, diet information, exercise information, exercise routines, treatment options, healthcare advise, wellness advice, preventive care, preventive procedure, health maintenance, drug and medication information, drug interaction information, video information, including video files or clips and other information, regarding illnesses, diseases, treatments and follow-up care, audio information, including audio files or clips and other information, regarding illnesses, diseases, treatments and follow-up care, treatment and/or procedure information and/or narratives, treatment analysis, diagnosis analysis, diagnosis monitoring, diagnosis confirmation and/or checking, and/or other information for providing the herein-described functions, services, and/or operations.

The database 10H can also contain information regarding the insurance companies and payers described herein, including, but not limited to, payer name, address, phone number, fax number, e-mail address, identification number(s), coverage types, policies and/or coverages provided, reimbursement rates, patients and/or providers serviced and/or covered by the payer, policy information, claim forms, claim procedures, claim status, claim processing information, claim submission procedures and policies, reasonable and customary charges, co-payment information, pre-approval information and/or procedures, claim form information, electronic form claim forms, insurance and/or coverage requirements, guidelines, and/or triggering events, covered procedures and/or treatments, uncovered procedures and/or treatments, claim approval information, claim approval history, claim approval statistics, claim rejection or denial information, claim rejection or denial history, claim rejection or denial statistics, financial account information, network provider information, network patient information, claim statistics, preventative care and/or benefits information, benefits information, benefits request information and/or claim forms, claim submission information, claim processing information, claim status information, payment information and statistics, and/or any other data and/or information regarding and/or related to payers which are needed and/or desired for providing any and/or all of the functions, services, and/or operations described herein.

The database 10H can also contain data and/or information regarding the brokers, agents and/or intermediaries described herein, including, but not limited to, intermediary name, address, phone number, fax number, e-mail address, clients, patients services, insurance policies, policy information, policy quote information, policy proposal information, any and/or all of the above information described herein regarding patients, providers, payers, etc. which may be of interest to the intermediaries described herein which may be useful and/or beneficial to the intermediaries in providing any of the functions, services, and/or operation described herein.

The database 10H can also contain contact information such as phone numbers, fax numbers, pager numbers, beeper numbers, e-mail addresses, hyperlinks to, and/or any other information which can facilitate contact between any of the parties described herein. The database 10H also includes electronic signature data and/or information for any of the parties, patients, providers, payers, and/or intermediaries, described herein for facilitating transactions, claim submissions, financial transactions, etc., by and/or between any of the above patients, providers, payers, and/or intermediaries.

The database 10H can also contain and notes, comments, or messages, which can be provided by any user, individual, patient, caregiver, provider, insurer, payer, third party, intermediary, or any other person or entity who or which utilizes the apparatus 100 and method of the present invention.

The database 10H can also contain any number of electronic healthcare records, electronic medical records, electronic dental records, electronic pharmacy records, electronic behavioral health records, and/or links to other electronic healthcare records, electronic medical records, electronic dental records, electronic pharmacy records, electronic behavioral health records which might be located in healthcare records computers 60 or other computers or computer systems located remote from the central processing computer 10. Any and/or all of these electronic healthcare records, electronic medical records, electronic dental records, electronic pharmacy records, electronic behavioral health records can contain data and/or information and/or link or hyperlinks to data and/or information regarding any individuals, patients, his or her caregivers, children, parent, relatives, friends, social networking friends, connections, of followers, providers, insurers or payers. The databases 10H can also contain any and/or all information regarding the data and/or information fields in each electronic healthcare record, electronic medical record, electronic dental record, electronic pharmacy record, and/or electronic behavioral health record, information for mapping common or like data fields for each and information or links or hyperlinks for accessing like data or like information fields in each of the different electronic healthcare records, electronic medical records, electronic dental records, electronic pharmacy records, electronic behavioral health records.

The data and/or information in the database 10H can also include links to any other information, information sources, news sources, and/or other information and/or data which can or may be utilized by the present invention and/or by any of the patients, providers, payers, intermediaries and/or any other users of the present invention.

The database 10H can also contain data and/or information regarding healthcare news, healthcare developments, healthcare discoveries, etc., for and including the medical field, surgical field, psychological field, dental field, nutrition field, fitness field, etc., and/or any other healthcare field or fields.

The database 10H, in the preferred embodiment, can also contain video and/or audio files which can be utilized for training of healthcare professionals as well as for providing general information to any user of the present invention. In this manner, and as will be described hereinbelow, the apparatus 100 can be utilized as a simulator for providing training in medical diagnosing, medical training, surgical training, psychiatric training, psychological training, dental training, oral surgery training, therapist training, and/or for training any of the healthcare providers described herein and/or envisioned.

For example, the present invention can be utilized to provide a medical doctor with a set of symptoms, evaluate the diagnosis and treatment prescribed and provide follow-up patient conditions which may or may not call for the medical doctor to re-evaluate his or her diagnosis and/or treatment. In a similar fashion, the present invention can be used for training and continuing education and training for any of the healthcare providers described herein and/or otherwise envisioned utilizing the present invention.

The database 10H can also contain data and/or information restricting access by any of the providers, payers, patients, intermediaries, and/or other users, to any of the data and/or information stored in the database 10H.

The database 10H can also contain information correlating symptoms and/or conditions with diagnoses, prognoses, and/or treatments, treatment methods, procedures, etc. The database 10H also contains any and/or all information needed and/or desired for facilitating the processing of symptoms, conditions, medical histories, family histories, and other information, in order to arrive at diagnoses and/or prognoses, treatments, prescriptions, procedures and/or any other healthcare and/or healthcare-related information.

The database 10H can also contain information regarding medical records, medical charts, x-rays, MRIs, and/or any other information, described herein and/or otherwise, which can be associated with an individual's or patient's medical history, medical record, or medical file, for any individual or patient who utilizes the apparatus 100 and method of the present invention.

The database 10H can also contain information regarding surgical records, surgical charts, x-rays, MRIs, and/or any other information, described herein and/or otherwise, which can be associated with an individual's or patient's surgical history, surgical record, or surgical file, for any individual or patient who utilizes the apparatus 100 and method of the present invention.

The database 10H can also contain information regarding dental records, dental charts, x-rays, and/or any other information, described herein and/or otherwise, which can be associated with an individual's or patient's dental history, dental record, or dental file, for any individual or patient who utilizes the apparatus 100 and method of the present invention.

The database 10H can also contain information regarding a prescription drug record, file, or account, for an individual or patient for any individual or patient who utilizes the apparatus 100 and method of the present invention.

The database 10H can also contain information regarding a healthcare spending account or healthcare spending accounts for any individual or patient, or for any of the individuals or patients, described herein or otherwise, who utilize the apparatus 100 and method of the present invention. For example, the healthcare spending account is or can be an account which can be a deposit account, a credit account, a debit account, an electronic money or an electronic cash account, or any combination of same, which can be held by any individual, patient, employer, bank, credit union, financial institution, investment institution, healthcare insurer, healthcare payer, or any other third party, and which can be used as a source of funds for payment for any type of healthcare service or healthcare product which is provided to, or administered to, or sought by, any individual or patient who utilizes the apparatus 100 and method of the present invention. In a preferred embodiment, the healthcare spending account can be used by the individual or patient to pay for any healthcare service, treatment, procedure, or product, at any time a payment is desired to be made for same.

The database 10H can also contain, for each individual or patient who utilizes the apparatus 100 and method of the present invention, information regarding, or a record of, each and every instance of a provider office visit involving the individual or patient, a communication(s) with a provider or providers involving or regarding the individual or patient, a payment(s) made by or on behalf of the individual or patient, an insurance claim(s) made by a provider or providers on a healthcare insurance policy or account for or regarding the individual or patient, including information regarding date of claim, time of claim, or the provider making the claim, a co-payment(s) made by or on behalf of the individual or patient, a payment(s) made on an insurance claim, a denial(s) of an insurance claim or insurance claims, or a resolution(s) of an insurance claim or insurance claims. The database 10H can also contain information regarding a history or record of insurance claims made by a provider or providers, or made by an individual or patient, for each individual or patient who utilizes the apparatus 100 and method of the present invention.

The database 10H can also contain credit report data or information, a credit report, credit reports, or a credit history, for any individual or patient, or for any of the individuals or patients, described herein or otherwise, who utilize the apparatus 100 and method of the present invention. The credit report or credit reports, or credit history, can contain any of the information which is typically provided by, or which may be provided by, any commercial or other credit reporting company or agency, such as, for example, but not limited to, credit reporting companies such as TransUnion LLC, Experian Information Solutions, Inc., Equifax, Inc., or any other credit reporting company, agency, or bureau, or any credit card company or financial institution. The database 10H can also contain any data or information which is typically found in a credit report database or credit history database, and/or any data and/or information which is, or which can be, utilized in generating, and/or analyzing, a credit report or credit reports, or a credit history, for, or on behalf of, any individual or patient, or for any of the individuals or patients, described herein or otherwise, who utilize the apparatus 100 and method of the present invention. The database 10H can also contain e-mail contact information, telephone contact information, cellular telephone contact information, and/or any other suitable contact information, for an individual or patient which can be utilized to notify the individual or patient when information regarding his or her credit report or credit reports, or credit history, is accessed, obtained, changed, or when a new entry of information is made in same.

The database 10H can also include advertisements, advertisement materials and information, marketing materials or information, commercials, video clips, infomercials, and any other information, which can include text information. video information, audio information, or any combination of same, which can be used to provide an advertisement, or advertisement material or information, to or for any individual, patient, provider, healthcare provider, payer, healthcare payer, insurer, healthcare insurer, product provider, service provider, third party provider, or any other person or entity, who or which utilizes the apparatus 100 and method of the present invention. A respective advertisement, advertisement material and information, marketing material or information, commercial, video clips, infomercials, and/or any other information, can be used to advertise or market any product(s) or service(s) which can be used by, provided to, provided by, advertised to, advertised by, marketed to, or marketed by, any individual, patient, provider, healthcare provider, payer, healthcare payer, insurer, healthcare insurer, product provider, service provider, third party provider, or any other person or entity, who or which utilizes the apparatus 100 and method of the present invention.

The database 10H can also contain news information, news reports, published reports, theses, study reports, or any other data or information regarding health issues, healthcare issues, health conditions, diseases, treatments, drugs, medications, healthcare procedures, medical procedures, surgical procedures, dental procedures, prescription products or services, non-prescription products or services, over-the-counter products or services, health or wellness issues or recommendations, exercise issues or recommendations, alternate therapy or treatment types or recommendations, herbal therapies or recommendations, or any other data or information which can be used by or provided to any individual, patient, provider, healthcare provider, payer, healthcare payer, insurer, healthcare insurer, product provider, service provider, third party provider, or any other person or entity, who or which utilizes the apparatus 100 and method of the present invention. The news information, news reports, published reports, theses, study reports, or any other data or information can include text information, video information, audio information, or any combination of same.

The news information, news reports, published reports, theses, study reports, or any other data or information, stored in the database 10H, can be transmitted to, or fed to, the central processing computer 10, for storage in the database 10H, by or from any appropriate computer or communication device, which is associated with or used by any news source, news organization, research institution, research professional, healthcare facility, an author, researcher, academician, or any other individual, person, or entity. An intermediary communication device 50 can also be utilized to transmit the news information, news reports, published reports, theses, study reports, or any other data or information to the central processing computer 10.

The database 10H can also contain prescription drugs information, prescription medication information, over-the-counter drug or medication information, wellness information, exercise and fitness information, and/or any other information pertinent to an individual's or a patient's heath, well-being, or treatment.

The database 10H can also include language translation information or software so that any of the data or information described herein as being stored in the database 10H, or as being provided in any of the messages, reports, or other communications, described herein can be translated into any language.

The database 10H can also contain, for each individual, patient, provider, and/or insurer or payer who or which utilize the apparatus 100, data and/or information regarding charges made to an individual or patient, charges made by a provider, charges made by an insurer or payer, amounts paid to an individual or patient, amounts paid by an individual or patient to a provider, amounts paid by an insurer or payer to a provider, amounts paid by an insurer or patient to an individual or patient, amounts paid by an individual or patient to an insurer or payer, a charges-back(s) made regarding a payment to, for, or by, a provider, a charge-back(s) made regarding a payment to, for, or by, an individual or patient, and/or a charge-back(s) made regarding a payment to, for, or by, an insurer.

The database 10H can also contain any notes, comments, or messages, which can be provided in text, in audio recordings or audio clips, and/or in video recordings or video clips. The database 10H can also contain any data, information, and/or software programs for translating audio information to text information and for translating test information to audio information. The database 10H can also contain any data, information, and/or software programs for translating text information or audio information from one language to another so as to provide a multi-lingual healthcare information and records keeping system. The database 10H can also contain any notes, comments, or messages, which can be provided by any of the herein-described users, individuals, patients, providers, insurers, payers, third parties, intermediaries, and/or any other person or entity who or which utilizes the apparatus 100 and method of the present invention.

The database 10H can also contain any notes, comments, or messages, which can be stored in any and/or all of the electronic healthcare records or files, electronic medical records or files, electronic dental records or files, electronic pharmacy records or files, electronic behavioral health records or files, and/or personal health records or files, described herein as being utilized in connection with the apparatus 100 and method of the present invention. In this regard, it is important to note that, in a preferred embodiment of the present invention, any and/or all of the electronic healthcare records or files, electronic medical records or files, electronic dental records or files, electronic pharmacy records or files, electronic behavioral health records or files, and/or personal health records or files, described herein as being utilized in connection with the apparatus 100 are or can be designed to receive, store, and/or provide, any notes, comments, and/or messages, provided by any of the herein-described users, individuals, patients, providers, insurers, payers, third parties, intermediaries, and/or any other person or entity, as text information or in text form, as audio information or in audio form, or as video information or in video form. Any text or audio information can be translated to from text to audio or from audio to text, and any text or audio information can be translated from one language to any one of more of any other language.

The database 10H can also contain statistical and/or other probabilistic and/or mathematical information for assigning and/or correlating certain levels and/or estimates for any and/or all of the information, diagnoses, prognoses, treatments, procedures, and/or any other information processed and/or generated by the central processing computer 10 and/or the apparatus 100.

The database 10H, in the preferred embodiment, can be a database which may include individual databases or collections of databases, with each database being designated to store any and all of the data and/or information described herein. The database 10H, or collection of databases, may be updated by each of the respective patients, providers, payers, users, and/or intermediaries, and/or by any other third party, in real-time, and/or via dynamically linked database management techniques.

The data and/or information stored in the database 10H can also be updated by and/or dynamically linked to, various external sources, including but not limited to news services, research publications, research facilities, healthcare laboratories, providers of healthcare goods and/or services, pharmaceutical companies, research institutions, schools. The database 10H will contain any and all information deemed necessary and/or desirable for providing all of the processing and/or services and/or functions described herein.

The database 10H can also contain data and/or information regarding any and/or all International Classification of Diseases (ICD) codes, ICD-10 codes, ICD-9, or any other ICD codes, or other codes, billing codes, diagnostic codes, treatment codes, or symptom codes, as well as any other codes or coding information which pertains to healthcare, the administration of healthcare services, healthcare record keeping, healthcare billing, healthcare diagnostics, healthcare treatments, or any other healthcare or healthcare-related functions or services, or any information or function, described-herein as capable of being provided by the apparatus 100 and method of the present invention. The database 10H can also contain data and/or information regarding the foreign language translations or conversions of or for, or for the foreign language translation or converting, of any of the herein-described International Classification of Diseases (ICD) codes, ICD-10 codes, ICD-9, any other ICD codes, or other codes, billing codes, diagnostic codes, treatment codes, symptom codes, as well as any other codes or coding information which pertains to healthcare, the administration of healthcare services, healthcare record keeping, healthcare billing, healthcare diagnostics, healthcare treatments, or any of the healthcare or healthcare-related functions or services, or any information or function described-herein as capable of being provided by the apparatus 100 and method of the present invention, into any one or any number of foreign languages.

The database 10H can contain any look-up tables, or information for use in a look table, for translating or converting any of the data, information, or codes, described herein, into any one or any number of foreign languages. The database 10H can also contain any language translation software or language conversion software which can be used to perform any of the foreign language translation or foreign language conversion processing or functionality described as being performed by the apparatus 100 and method of the present invention.

The database 10H can also contain any information or software, or rules algorithms, which can be used in generating insurance claim forms which can be submitted error-free to any of the insurers or payers described herein.

The database 10H can also contain digital pictures, photographs, images, video, or video clips, of individuals or patients, as well as digital pictures, photographs, images, video, or video clips, of physical conditions of or regarding an individual or patient, which can be accessed or retrieved by any of the individuals, patients, providers, payers, or intermediaries, described herein as utilizing the apparatus 100 and method of the present invention. For example, in instances where photographs or images can be useful to see a state of a physical condition of a patient or individual, or to compare a state of a physical condition from one point in time to another, or to track the state of a physical condition, a photograph(s), picture(s), or image(s), or video clip, can be digitally recorded, with a camera or video recording device, and stored in the database 10H and/or in the patient's or individual's healthcare record or healthcare file for later retrieval, use, and/or review.

For example, a dermatologist seeking to determine if a skin condition has or is changing in size, shape, or color, can take and store, in the database 10H and/or in the patient's or individual's healthcare record or healthcare file, one or more photographs, pictures, or images, or a video clips, of the skin condition. In an analogous manner, a plastic surgeon can obtain and store, in the database 10H and/or in the patient's or individual's healthcare record or healthcare file, photographs, pictures, or images, or video clips, showing physical conditions of an individual or patient before, after, or during a procedure or procedures. A dentist or an oral health professional can obtain and store, in the database 10H and/or in the patient's or individual's healthcare record or healthcare file, in addition to x-rays, a photograph(s), picture(s), or image(s), or a video clip(s), showing physical conditions of an individual's or patient's mouth, teeth, gums, or other physical oral state, for later retrieval, use, or review. In an analogous manner, any healthcare provider can obtain and store, in the database 10H and/or in a patient's or individual's healthcare record or healthcare file, a photograph(s), picture(s), or image(s), or a video clip(s), obtained in or during an office visit, an examination, a procedure, an operation, a treatment, or during any other occasion, event, occurrence, or happening, for later retrieval and use as needed or desired by any provider, payer, patient, individual, or intermediary, described herein as being able to utilize the apparatus 100 and method of the present invention.

The database 10H can also contain video and/or audio clips or recordings of office visits, examinations, procedures, operations, or any other healthcare, healthcare-related, or any other event(s), occurrence(s), or happening(s), which may or which can be of interest to any provider, insurer, payer, patient, individual, or intermediary, described herein as being able to utilize the apparatus 100 and method of the present invention.

The database 10H can also contain transcripts of office visits, examinations, procedures, operations, or any other healthcare, healthcare-related, or any other event(s), occurrence(s), or happening(s), which may or which can be of interest to any provider, insurer, payer, patient, individual, or intermediary, described herein as being able to utilize the apparatus 100 and method of the present invention.

The database 10H can also contain data, information, links, or hyperlinks, which can allow a healthcare provider to create or generate and/or transmit prescriptions or electronic prescriptions, or prescription forms, to pharmacies or other providers, for a patient or individual, or prescriptions or electronic prescriptions for tests, analyses, analysis work-ups, procedures, therapy, physical therapy, or other healthcare or healthcare-related services, for a patient or individual, or which can allow a healthcare provider to transmit a referral or an electronic referral or referral form to other healthcare providers or other providers of goods or services for a patient or individual.

The database 10H can also contain data, information, links, or hyperlinks, which can allow a healthcare provider to perform remote control or monitoring of healthcare devices or equipment used in the treatment of, a care of, or a monitoring of, a patient or individual, while the patient or individual is at home, in a hospital, in a nursing home, in a care facility, or traveling in or on, respectively, a car, automobile, train, subway train, boat, helicopter, airplane, aircraft, or any other vehicle.

The database 10H can also contain data, information, or software, for performing healthcare audits, for performing meaningful use information processing routines, for identifying or selecting individuals for clinical trials, experimental procedures, experimental treatments, healthcare focus groups, healthcare surveys, or other activities or events, or for performing any other processing routines which can advantageously use any of the information stored in the database 10H, any information stored in any of the herein-described healthcare records, files, or histories, described herein, or any other information, stored by the apparatus 100 of the present invention or any of its computers, communication devices, component parts, or devices, or any other information stored in, provided by, generated by, or used by, the apparatus 100 of the present invention. For example, with regards to clinical trials, experimental procedures, experimental treatments, healthcare focus groups, healthcare surveys, or other activities or events, the database 10H can contain information regarding whether or not an individual or patient can or may be a candidate for, be eligible to participate in, has agreed to be candidate for, or has agreed to participate in, clinical trials, experimental procedures, experimental treatments, healthcare focus groups, healthcare surveys, or other activities or events, or has agreed to be candidate for, or has agreed to participate in, a particular clinical trial(s), experimental procedure(s), experimental treatment(s), healthcare focus group(s), healthcare survey(s), or other activities or events.

The database 10H can also contain any data and/or information needed or used to determine or measure the effectiveness of, or side effects experienced or associated with, any medication(s), medicine(s), drug(s), dietary supplement(s), supplement(s), vitamin(s), nutrient(s), over-the-counter product(s) or substance(s), or the effectiveness of a procedure(s), treatment(s), treatment plan(s), care management plan(s), or any other good(s) or service(s) which can be offered in the marketplace.

The database 10H can also contain data and/or information relating to information regarding health conditions, healthcare conditions, illnesses, diagnoses, symptoms, treatments, treatment plans, care management plans, care management practices, insurance information, information regarding type of insurance, wellness information, healthcare practices, healthcare history patterns, healthcare trends, treatment trends, care management trends, statistics, statistical analyses, studies, study trends, or any other healthcare or healthcare-related information, for or regarding any number or type of groups of individuals or patients, and any data or information for determining same. The database 10H can also contain data or information regarding, or for determining, any of the above-described information for any number of groups of individuals or patients based on sex, gender, age, occupation, education, ethnicity, nationality, country of origin, religion, or any demographic or demographics, or any other criteria. The database 10H can also contain data or information regarding, or for determining statistical information or probability information regarding, any of the herein-described information.

The database 10H can also contain data or information for implementing and for performing drug formulary checks or analyses, as well as data or information for performing medication, medicine, or drug, reconciliation.

The database 10H can also contain immunization data or information.

The database 10H can also contain educational or instructional information, links or hyperlinks to same, information regarding instructions or procedures, or links or hyperlinks to same, in order to allow for the providing of patient-specific educational or instructional information or resources to patients or individuals, as well as to any of the herein-described healthcare providers, other providers, payers or insurers, or intermediaries or caregivers.

The database 10H can also contain data or information necessary or desired for administering employee benefits for any of the individuals or patients who utilize the apparatus 100 and method of the present invention.

The database 10H can also contain data or information necessary or desired for purchasing or enrolling in employee benefits for any of the individuals or patients who utilize the apparatus 100 and method of the present invention.

The database 10H can also contain data or information necessary or desired for allowing any individual or patient to make a request or a claim for, or pursuant to, or to file a request or a claim for, an employee benefit or employee benefits, for any of the individuals or patients who utilize the apparatus 100 and method of the present invention.

The database 10H can also contain data or information for facilitating, or for providing for, the portability of healthcare insurance or health insurance benefits, life insurance or life insurance benefits, or disability insurance or disability insurance benefits, or for providing for the portability of any other employee benefit or employee benefits.

The database 10H can also contain any of the messages or reports described herein as being generated by or provided by the apparatus 100 and method of the present invention, for later use by any provider, insurer, payer, patient, individual, or intermediary, or any other person or entity, described herein as being able to utilize the apparatus 100 and method of the present invention.

The database 10H can also contain any data, information, or software, for performing any of the processing routines or functionality described herein as being capable of being performed by, or provided by, the apparatus 100 of the present invention.

The data and/or information which is contained and/or stored in the database 10H as well as any of the other databases 20H, 30H, 40, and 50H, described herein can be obtained from the various patients, individuals, providers, payers, and/or intermediaries, who or which utilize and/or who or which are serviced by the present invention. For example, the respective patients, providers, payers, intermediaries, and/or other users, could fill out questionnaires, forms, narratives, claim forms, and/or any other information medium, in written form, electronically, and/or otherwise.

Data and/or information stored in the database 10H as well as any of the other databases described herein can be updated by multiple parties. For example, a patient may provide a medical history for his or her individual file, his or her medical doctor can update the medical history information for the patient upon examining and/or treating him or her. The payer may also update the file with any associated payment or payment-related information. Should the patient go to another doctor or different type of doctor, all previous information would be available for, and can be updateable by, the next doctor.

The database 10H can also contain information regarding alternate medicine techniques, herbal techniques, meditation techniques, exercise techniques, self-healing, faith healing, and/or other non-medicine treatments and/or techniques.

The database 10H can also include statistical data and/or information regarding diagnoses, and/or alternate diagnoses, treatment success, treatment failure, as well as statistical data and/or information regarding misdiagnoses. The database 10H also contains data and/or information regarding experimental treatments as well as statistical information regarding same, successes of same and failures of same.

The database 10H can also contain any other data and/or information, including software for performing any and all of the operations, routines, processing routines, and other functions and/or functionality described herein as being capable of being performed by the apparatus 100, the central processing computer(s) 10 and any of the herein-described provider communication device(s) 20, payer communication device(s) 30, user or patient communication device(s) 40, intermediary communication device(s) 50, healthcare records computer(s) 60, electronic interactive content/media computers 70, insurance exchange computer(s) 80, social networking computer(s) 90, and/or media computer(s) 95.

The database 10H can also contain any and/or all of the herein-described information for providing electronic veterinary healthcare records or filed (EVHRs) which can be used for maintaining healthcare records or file for any types or kinds f animals, high value animals, exotic animals, pet of any kind or type, or any other types or kinds of animals. For example, electronic veterinary healthcare records or files can be maintained and used for race horses, circus animals, animals used in shows, service animals, animals used for breeding, household pets, dogs, cats, fish, birds, mammals of any kind or types, birds of any kinds or types, and/or any other animal for which an owner, caregiver, or other responsible person or entity, may find it desirable or necessary to keep a healthcare record or file. In a preferred embodiment, the database 10H can also contain, any of the data and/or information, or any analogous data and/or information, described herein as being stored or contained in the database 10H, for any animal for which an electronic veterinary healthcare record or file is maintained.

It is important to note that the apparatus 100 and method of the present invention can be utilized, in any and or all of the embodiments described herein, in order to process and/or to provide veterinary healthcare information and/or veterinary healthcare-related information for and regarding any kind or type of animal or animals.

The database 10H can also contain any data and/or information, as well as any links or hyperlinks to any of the herein-described provider communication device(s) 20, payer communication device(s) 30, user or patient communication device(s) 40, intermediary communication device(s) 50, healthcare records computer(s) 60, electronic interactive content/media computers 70, insurance exchange computer(s) 80, social networking computer(s) 90, and/or media computer(s) 95, described herein as being used in connection with the present invention, and/or can contain data and/or information and/or links or hyperlinks to any data and/or information located or stored on, at, or in any of the provider communication device(s) 20, payer communication device(s) 30, user or patient communication device(s) 40, intermediary communication device(s) 50, healthcare records computer(s) 60, electronic interactive content/media computer(s) 70, insurance exchange computer(s) 80, social networking computer(s) 90, and/or media computer(s) 95, described herein.

The database 10H can also contain information, links and/or hyperlinks, to any social networking web sites, web pages, support groups, on-line forums, on-link information services, as well as social networking web sites or social networking web pages to or for social networking members, support groups, information providers, healthcare providers, as well as any of the providers, insurers, payers, individuals, patient, third parties, intermediaries, or any other persons or entities described herein who are or who may be members of any social network.

In any and/or all of the embodiments described herein, any of the data and/or information which is or which may be stored in the database 10H, and/or any of the other databases described herein, can be utilized and/or can appear in any of the reports, diagnostic reports, treatment reports, evaluation reports, provider reports, payer reports, patient reports, training reports, and/or any other reports, described herein.

The central processing computer 10 also includes an output device 10I for outputting any data, information, report, etc., described herein. In the preferred embodiment, the output device 10I can be a printer, a display, a transmitter, a modem, and/or any other device which can be used to output data.

Any of the data and/or information for any of the patients, individuals, providers, payers, and/or intermediaries, can be updated by different parities and which such updated data and/or information being made available to other respective parties so as to provide and ensure comprehensive and up-to-date healthcare and healthcare-related information.

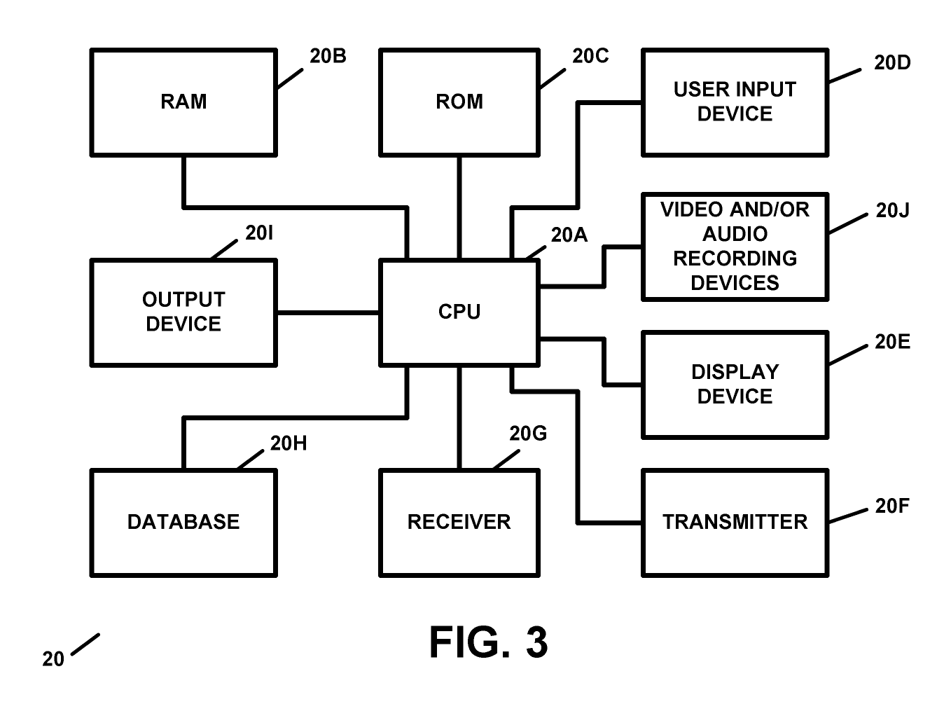

FIG. 3 illustrates the provider communication device 20, in block diagram form. The provider communication device 20, in the preferred embodiment, can be personal computer, a network computer or computer system, or any other computer or communication device, which is utilized as a provider computer 20. In the preferred embodiment, the provider communication device 20 includes a central processing unit or CPU 20A, which in the preferred embodiment, is a microprocessor. The CPU 20A may also be a microcomputer, a minicomputer, a macro-computer, and/or a mainframe computer, depending upon the application.

The provider communication device 20 also includes a random access memory device(s) 20B (RAM) and a read only memory device(s) 20C (ROM), each of which is connected to the CPU 20A, a user input device 20D, for entering data and/or commands into the provider communication device 20, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, for example pulse rate monitors, blood pressure monitors, electrocardiograms, blood-sugars monitors, etc., if desired, which input device(s) are also connected to the CPU 20A.

The user input device 20D can also acquire, receive, generate and/or provide, data which can be entered by a user or individual and/or can be a device which can acquire, receive, generate and/or provide, digital data such as medical, healthcare, bio-metric, physiological, and/or any other kind of healthcare data and/or healthcare-related data. For example, a user input device 20D can be a keyboard for allowing a user to input information into the provider communication device and/or a user input device can, for example, by a heart rate monitor or EKG machine which can receive information from a patient or individual and generate a digital signal, digital data, analog data, and/or any other signal, data, and/or information, which is representative of the patient's or individual's heart rate, pulse rate, and/or cardiac activity.

The user input device 20D can also be, or can include, any one or more of, and/or any combination of, a thermometer, a digital thermometer, a stethoscope, a heart rate monitor or measurement device, a pulse rate monitor or measurement device, a blood pressure monitor or measurement device, a blood pressure measurement device, blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (Mill) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, a PET scan device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotrachial cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, any scope having a camera and/or a light, a laparascopic device, a laparascopic camera device, a camera used in or associated with equipment for performing visual probing of the body or any portions, parts, or organs, thereof, such as, but not limited to, a colonoscopy, an endoscopy, an esophagoscopy, a gastroscopy, or any devices used in laparascopic surgery or other surgeries, an ingestible or implantable pill, capsule, or device, containing a camera and/or a light and equipment or circuitry for transmitting information as well as images to equipment inside or outside the body, and/or any other bio-metric or physiological data measuring device(s) and/or data acquisition device (hereinafter referred to generally or collectively as “healthcare equipment input device”, “healthcare measurement input device”, or “healthcare monitoring input device”).

The user input device 20D can also be, or can include, a pacemaker, a device for monitoring blood pressure or blood flow, or an ingestible or implantable pill, capsule, or device, containing a camera and/or a light and equipment or circuitry for transmitting information as well as images to equipment inside or outside the body.

The provider communication device 20 also includes a display device 20E for displaying data and/or information to a user or operator.

The provider communication device 20 also includes a transmitter(s) 20F, for transmitting signals and/or data and/or information to any one or more of the central processing computer(s) 10, any other provider communication device(s) 20, the communication device(s) 30, the user or patient communication device(s) 40, the intermediary computer(s) 50, the healthcare records computer(s) 60, the insurance exchange computer(s) 80, the social networking computer(s) 90, and/or the media computer(s) 95, described herein, which may be utilized in conjunction with the present invention. The provider communication device 20 also includes a receiver 20G, for receiving signals and/or data and/or information from any one or more of the central processing computer(s) 10, any other provider communication device(s) 20, the payer communication device(s) 30, the user or patient communication device(s) 40, the intermediary computer(s) 50, the healthcare records computer(s) 60, the insurance exchange computer(s) 80, the social networking computer(s) 90, and/or the media computer(s) 95, described herein, which may be utilized in conjunction with the present invention.

The provider communication device 20 also includes a database(s) 20H. The database 20H can contain and/or be linked to any of the data and/or information described herein as being stored in the database 10H.

The provider communication device 20 also includes an output device 201 for outputting any data, information, report, etc., described herein. In the preferred embodiment, the output device 201 can be a printer, a display, a transmitter, a modem, and/or any other device which can be used to output data.

The provider communication device 20 also includes a video and/or audio recording device(s) 20J which can include a camera and/or a video recording device for recording pictures or video and/or video clips and/or a microphone or an audio recording device for recording audio or audio clips which can be stored in any of the electronic healthcare, electronic medical, electronic dental, electronic pharmacy, and/or electronic behavioral healthcare, records or files described herein, and/or which can be otherwise used for communicating with other users, individuals, patients, providers, payers, or persons or entities who or which utilize the apparatus 100 and method of the present invention.

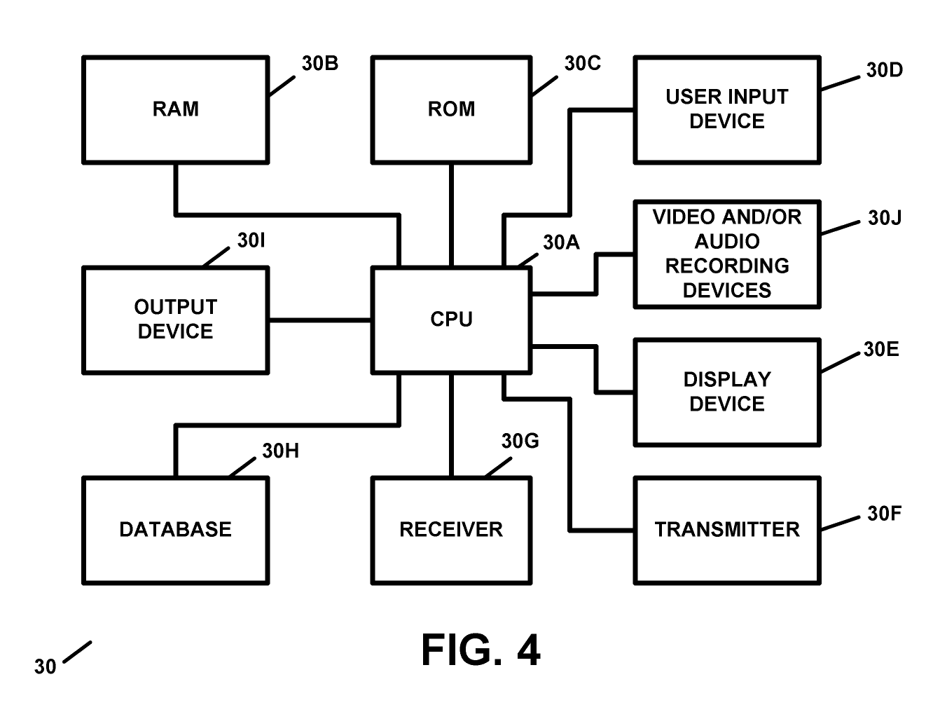

FIG. 4 illustrates the payer communication device 30, in block diagram form. The payer communication device 30, in the preferred embodiment, can be personal computer, a network computer or computer system, or any other computer or communication device, which is utilized as a payer computer 30. In the preferred embodiment, the payer communication device 30 includes a central processing unit or CPU 30A, which in the preferred embodiment, is a microprocessor. The CPU 30A may also be a microcomputer, a minicomputer, a macro-computer, and/or a mainframe computer, depending upon the application.

The payer communication device 30 also includes a random access memory device(s) 30B (RAM) and a read only memory device(s) 30C (ROM), each of which is connected to the CPU 30A, a user input device 30D, for entering data and/or commands into the payer communication device 30, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, for example pulse rate monitors, blood pressure monitors, electro-cardiograms, blood-sugars monitors, etc., if desired, which input device(s) are also connected to the CPU 30A. The payer communication device 30 also includes a display device 30E for displaying data and/or information to a user or operator.

The payer communication device 30 also includes a transmitter(s) 30F, for transmitting signals and/or data and/or information to any one or more of the central processing computer(s) 10, the provider communication device(s) 20, any other payer communication device(s) 30, the user or patient communication device(s) 40, and the intermediary computer(s) 50, the healthcare records computer(s) 60, the insurance exchange computer(s) 80, the social networking computer(s) 90, and/or the media computer(s) 95, described herein, which may be utilized in conjunction with the present invention. The payer communication device 30 also includes a receiver 30G, for receiving signals and/or data and/or information from any one or more of the central processing computer(s) 10, the provider communication device(s) 20, any other payer communication device(s) 30, the user or patient communication device(s) 40, the intermediary computer(s) 50, the healthcare records computer(s) 60, the insurance exchange computer(s) 80, the social networking computer(s) 90, and/or the media computer(s) 95, described herein, which may be utilized in conjunction with the present invention.

The payer communication device 30 also includes a database(s) 30H. The database 30H can contain and/or be linked to any of the data and/or information described herein as being stored in the database 10H.

The payer communication device 30 also includes an output device 301 for outputting any data, information, report, etc., described herein. In the preferred embodiment, the output device 301 can be a printer, a display, a transmitter, a modem, and/or any other device which can be used to output data.

The payer communication device 30 also includes a video and/or audio recording device(s) 30J which can include a camera and/or a video recording device for recording pictures or video and/or video clips and/or a microphone or an audio recording device for recording audio or audio clips which can be stored in any of the electronic healthcare, electronic medical, electronic dental, electronic pharmacy, and/or electronic behavioral healthcare, records or files described herein, and/or which can be otherwise used for communicating with other users, individuals, patients, providers, payers, or persons or entities who or which utilize the apparatus 100 and method of the present invention.

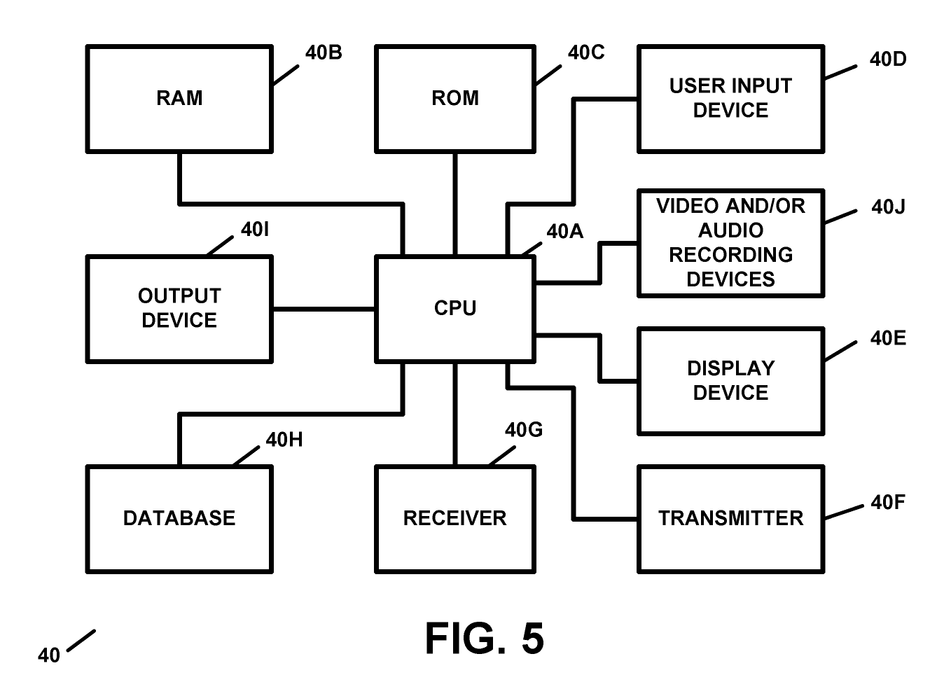

FIG. 5 illustrates the user or patient communication device 40, in block diagram form. The user or patient communication device 40, in the preferred embodiment, can be personal computer, a network computer or computer system, or any other computer or communication device, which is utilized as a patient computer. In the preferred embodiment, the user or patient communication device 40 includes a central processing unit or CPU 40A, which in the preferred embodiment, is a microprocessor. The CPU 40A may also be a microcomputer, a minicomputer, a macro-computer, and/or a mainframe computer, depending upon the application.

The user or patient communication device 40 also includes a random access memory device(s) 40B (RAM) and a read only memory device(s) 40C (ROM), each of which is connected to the CPU 40A, a user input device 40D, for entering data and/or commands into the user or patient communication device 40, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, for example pulse rate monitors, blood pressure monitors, electro-cardiograms, blood-sugars monitors, etc., if desired, which input device(s) are also connected to the CPU 40A.

The user input device 40D can also acquire, receive, generate and/or provide, data which can be entered by a user or individual and/or can be a device which can acquire, receive, generate and/or provide, digital data such as medical, healthcare, bio-metric, physiological, and/or any other kind of healthcare data and/or healthcare-related data. For example, a user input device 40D can be a keyboard for allowing a user to input information into the patient communication device and/or a user input device can, for example, by a heart rate monitor or EKG machine which can receive information from a patient or individual and generate a digital signal, digital data, analog data, and/or any other signal, data, and/or information, which is representative of the patient's or individual's heart rate, pulse rate, and/or cardiac activity.

The user input device 40D can also be, or can include, any one or more of, and/or any combination of, a thermometer, a digital thermometer, a stethoscope, a heart rate monitor or measurement device, a pulse rate monitor or measurement device, a blood pressure monitor or measurement device, a blood pressure measurement device, a blood analysis device or machine, a respirator, a respiration monitoring or measurement device, a dialysis machine, a dialysis device, electrocardiograph (EKG) machine or device, electrocephalograph (EEG) machine or device, electromyograph (EMG) machine or device, a magnetic resonance imaging (Mill) machine or device, X-ray machine or device, medical imaging machine or device, thermal imaging machine or device, a heart sound monitor or measurement device, a lung sound monitoring or measurement device, respiration rate monitoring or measurement device, a laparascopic device, an arthroscopic device, a vascular testing device, a catheter device, a cardiac performance testing, monitoring, or measurement device, a pulmonary performance testing, monitoring, or measurement device, a vascular system performance monitoring or measurement device, a vascular system testing, monitoring, or measurement device, a metabolism monitoring or measurement device, a sonogram imaging device, a sonogram measurement device, a sonograph device, an optical response device, an optical response measurement device, an intravenous device, an arterial blood pressure measurement or monitoring device, a respiration rate measurement or monitoring device, an ultrasound imaging device, an ultrasound measurement device, a CAT SCAN device, an optical metabolism measurement or monitoring device, a radiotelemetric device, a doppler medical device, a mammogram device, a carbon dioxide detection or measurement device, a carbon monoxide detection or measurement device, a transvascular impedance measurement or monitoring device, an ultrasonic imaging device, a bone conduction device, a brain function scan analyzer device, external pulse cardiac monitoring or measurement device, a fetal heart rate measurement, monitoring, or probing, device, an endotrachial cardiac monitoring device, a finger tip blood pressure monitoring device, a psychological monitoring device, a surgical instrument, a vital signs measurement device, ear pressure regulating device, phonocardiograph device, acoustic aneurysm detector device, blood oxygen detection device, esophageal probing device, ultrasonic probing device, ausculoscope, vital signs monitoring system, heart activity monitoring device, pulmonary activity monitoring device, sphygmomanometer, esophageal stethoscope, venous pressure measuring device, differential doppler device, physiological data measuring device, body tissue movement device, breathalyzer device, camera probing device, microscopic camera probing device, and/or any other bio-metric or physiological data measuring device(s) and/or data acquisition device data acquisition device (hereinafter referred to generally or collectively as “healthcare equipment input device”, “healthcare measurement input device”, or “healthcare monitoring input device”).

The user or patient communication device 40 also includes a display device 40E for displaying data and/or information to a user or operator.

The user or patient communication device 40 also includes a transmitter(s) 40F, for transmitting signals and/or data and/or information to any one or more of the central processing computer(s) 10, the provider communication device(s) 20, the payer communication device(s) 30, any other user or patient communication device(s) 40, and the intermediary computer(s) 50, the healthcare records computer(s) 60, the insurance exchange computer(s) 80, the social networking computer(s) 90, and/or the media computer(s) 95, described herein, which may be utilized in conjunction with the present invention. The user or patient communication device 40 also includes a receiver 40G, for receiving signals and/or data and/or information from any one or more of the central processing computer(s) 10, the provider communication device(s) 20, the payer communication device(s) 30, any other user or patient communication device(s) 40, the intermediary computer(s) 50, the healthcare records computer(s) 60, the insurance exchange computer(s) 80, the social networking computer(s) 90, and/or the media computer(s) 95, described herein, which may be utilized in conjunction with the present invention.

The user or patient communication device 40 also includes a database(s) 40H. The database 40H can contain and/or be linked to any of the data and/or information described herein as being stored in the database 10H.

The user or patient communication device 40 also includes an output device 401 for outputting any data, information, report, etc., described herein. In the preferred embodiment, the output device 401 can be a printer, a display, a transmitter, a modem, and/or any other device which can be used to output data.

The user or patient communication device 40 also includes a video and/or audio recording device(s) 40J which can include a camera and/or a video recording device for recording pictures or video and/or video clips and/or a microphone or an audio recording device for recording audio or audio clips which can be stored in any of the electronic healthcare, electronic medical, electronic dental, electronic pharmacy, and/or electronic behavioral healthcare, records or files described herein, and/or which can be otherwise used for communicating with other users, individuals, patients, providers, payers, or persons or entities who or which utilize the apparatus 100 and method of the present invention.

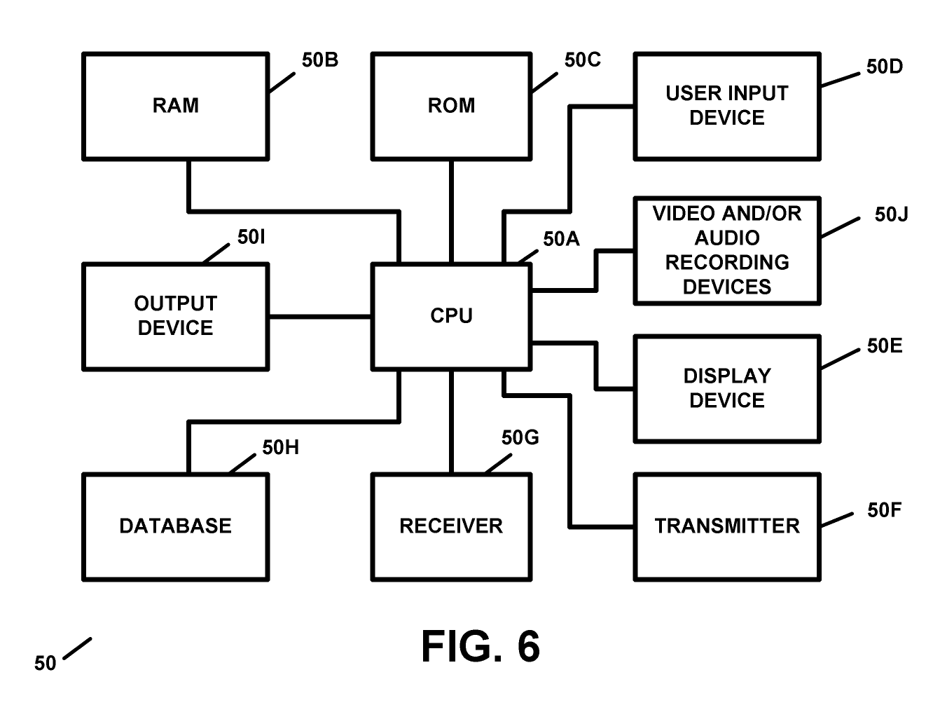

FIG. 6 illustrates the intermediary computer 50, in block diagram form. The intermediary computer 50, in the preferred embodiment, can be personal computer, a network computer or computer system, or any other computer or communication device, which is utilized as an intermediary computer 50. In the preferred embodiment, the intermediary computer 50 includes a central processing unit or CPU 50A, which in the preferred embodiment, is a microprocessor. The CPU 50A may also be a microcomputer, a minicomputer, a macro-computer, and/or a mainframe computer, depending upon the application.

The intermediary computer 50 also includes a random access memory device(s) 50B (RAM) and a read only memory device(s) 50C (ROM), each of which is connected to the CPU 50A, a user input device 50D, for entering data and/or commands into the intermediary computer 50, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, for example pulse rate monitors, blood pressure monitors, electrocardiograms, blood-sugars monitors, etc., if desired, which input device(s) are also connected to the CPU 50A. The intermediary computer 50 also includes a display device 50E for displaying data and/or information to a user or operator.

The intermediary computer 50 also includes a transmitter(s) 50F, for transmitting signals and/or data and/or information to any one or more of the central processing computer(s) 10, the provider communication device(s) 20, the payer communication device(s) 30, the user or patient communication device(s) 40, any other intermediary computer(s) 50, the healthcare records computer(s) 60, the insurance exchange computer(s) 80, the social networking computer(s) 90, and/or the media computer(s) 95, described herein, which may be utilized in conjunction with the present invention. The intermediary computer 50 also includes a receiver 50G, for receiving signals and/or data and/or information from any one or more of the central processing computer(s) 10, the provider communication device(s) 20, the payer communication device(s) 30, the user or patient communication device(s) 40, any other intermediary computer(s) 50, the healthcare records computer(s) 60, the insurance exchange computer(s) 80, the social networking computer(s) 90, and/or the media computer(s) 95, described herein, which may be utilized in conjunction with the present invention. which may be utilized in conjunction with the present invention.

The intermediary computer 50 also includes a database(s) 50H. The database 50H can contain and/or be linked to any of the data and/or information described herein as being stored in the database 10H.

The intermediary communication device 50 also includes an output device 501 for outputting any data, information, report, etc., described herein. In the preferred embodiment, the output device 501 can be a printer, a display, a transmitter, a modem, and/or any other device which can be used to output data.

The intermediary communication device 50 also includes a video and/or audio recording device(s) 50J which can include a camera and/or a video recording device for recording pictures or video and/or video clips and/or a microphone or an audio recording device for recording audio or audio clips which can be stored in any of the electronic healthcare, electronic medical, electronic dental, electronic pharmacy, and/or electronic behavioral healthcare, records or files described herein, and/or which can be otherwise used for communicating with other users, individuals, patients, providers, payers, or persons or entities who or which utilize the apparatus 100 and method of the present invention.

In any and/or all of the embodiments described herein, any one of the central processing computers 10, the provider communication devices 20, the payer communication devices 30, the patient communication devices 40, and/or the intermediary communication devices 50, can include input devices (not shown) for facilitating the data entry of a patients vital signs and or other medical data such as, but not limited to, pulse rate, blood pressure, blood-sugar level, etc., and any other data and or information which can be input into the respective computer and/or communication device and be transmitted to the central processing computer consistent with the utilization of the present invention as described herein.

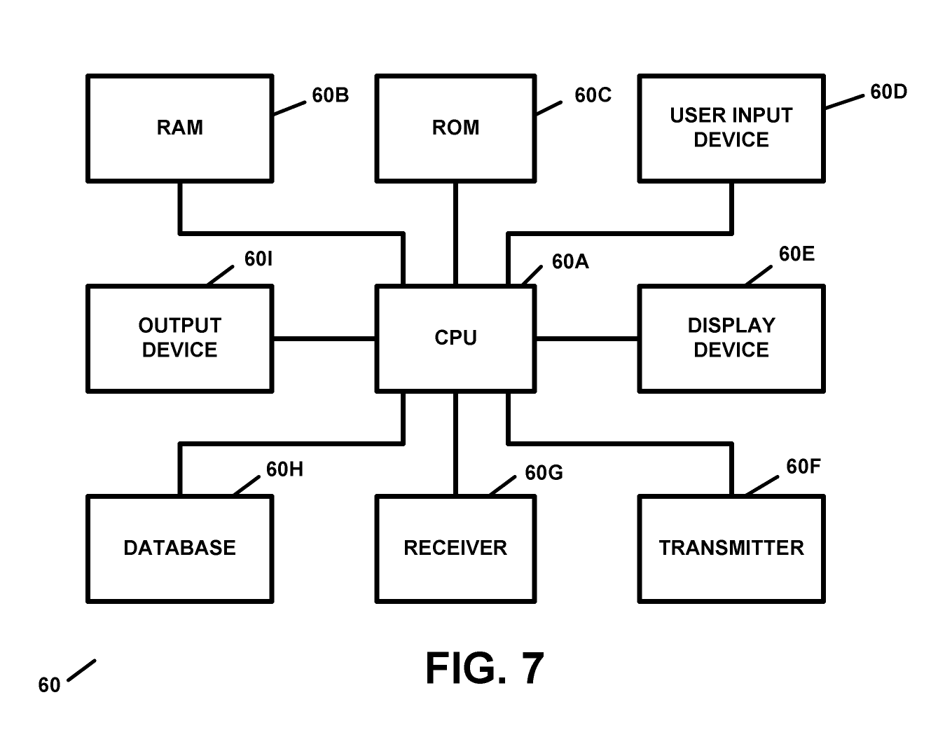

FIG. 7 illustrates the healthcare records computer 60, in block diagram form. The healthcare records computer 60, in the preferred embodiment, can be server computer or server computer system, a cloud computer or cloud computer system, a network computer or computer system, a personal computer or any other computer or communication device, or any cellular telephone, tablet, personal digital assistant, wireless communication device or wireless computer, or any other communication device, which is utilized to store an electronic healthcare record, an electronic medical record, an electronic dental record, an electronic pharmacy record, or an electronic behavioral healthcare record or any one or any number of electronic healthcare records, electronic medical records, electronic dental records, electronic pharmacy records, and/or electronic behavioral healthcare records. In a preferred embodiment, the healthcare records computer 60 can be any computer or computer system for storing any an electronic healthcare record, an electronic medical record, an electronic dental record, an electronic pharmacy record, or an electronic behavioral healthcare record which is store somewhere else other than the central processing computer. The healthcare records computer 60 can be used by, maintained by, or associated with any individual, provider, user, patient, payer, intermediary, or any other entity or third party.

In the preferred embodiment, the healthcare records computer 60 includes a central processing unit or CPU 60A, which in the preferred embodiment, is a microprocessor. The CPU 60A may also be a microcomputer, a minicomputer, a macro-computer, and/or a mainframe computer, depending upon the application.

The healthcare records computer 60 also includes a random access memory device(s) 60B (RAM) and a read only memory device(s) 60C (ROM), each of which is connected to the CPU 60A, a user input device 60D, for entering data and/or commands into the healthcare records computer 60, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information which input device(s) are also connected to the CPU 60A. The healthcare records computer 60 also includes a display device 60E for displaying data and/or information to a user or operator.

The healthcare records computer 60 also includes a transmitter(s) 60F, for transmitting signals and/or data and/or information to any one or more of the central processing computer(s) 10, the provider communication device(s) 20, the payer computers(s) 30, the user or patient communication device(s) 40, and the intermediary computer(s) 50, any other healthcare records computer(s) 60, the insurance exchange computer(s) 80, the social networking computer(s) 90, and/or the media computer(s) 95, described herein, which may be utilized in conjunction with the present invention. The healthcare records computer 60 also includes a receiver 60G, for receiving signals and/or data and/or information from any one or more of the central processing computer(s) 10, the provider communication device(s) 20, the payer computers(s) 30, the user or patient communication device(s) 40, the intermediary computer(s) 50, any other healthcare records computer(s) 60, the insurance exchange computer(s) 80, the social networking computer(s) 90, and/or the media computer(s) 95, described herein, which may be utilized in conjunction with the present invention.

The healthcare records computer 60 also includes a database(s) 60H. The database 60H can contain and/or be linked to any of the data and/or information described herein as being stored in the database 10H and can also contain any data and/or information regarding and including any of the of electronic healthcare records, electronic medical records, electronic dental records, electronic pharmacy records, and/or electronic behavioral healthcare records which are stored in the healthcare records computer 60.

The healthcare records computer 60 also includes an output device 601 for outputting any data, information, report, etc., described herein. In the preferred embodiment, the output device 601 can be a printer, a display, a transmitter, a modem, and/or any other device which can be used to output data.

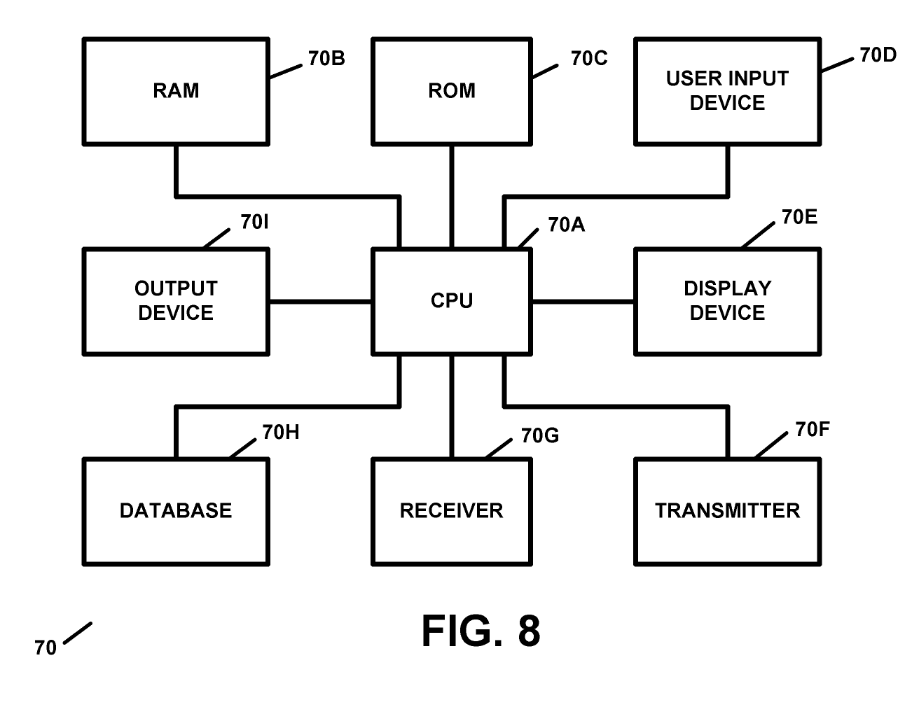

FIG. 8 illustrates the electronic interactive content/media computer 70, in block diagram form. The electronic interactive content/media computer 70, in the preferred embodiment, can be server computer or server computer system, a cloud computer or cloud computer system, a network computer or computer system, a personal computer or any other computer or communication device, or any cellular telephone, tablet, personal digital assistant, wireless communication device or wireless computer, or any other communication device, which is utilized to store electronic interactive content or media which can be provided by or supplied by any electronic interactive content or media provider. In a preferred embodiment, the electronic interactive content/media computer 70 can be any computer or computer system for storing any of the herein-described electronic interactive content or media described herein as being utilized in connection with the apparatus 100 of the present invention. The electronic interactive content/media computer 70 can be used by, maintained by, or associated with, any provider, supplier, or vendor, of any electronic interactive content or media, or any other entity or third party.

In the preferred embodiment, the electronic interactive content/media computer 70 includes a central processing unit or CPU 70A, which in the preferred embodiment, is a microprocessor. The CPU 70A may also be a microcomputer, a minicomputer, a macro-computer, and/or a mainframe computer, depending upon the application.

The electronic interactive content/media computer 70 also includes a random access memory device(s) 70B (RAM) and a read only memory device(s) 70C (ROM), each of which is connected to the CPU 70A, a user input device 70D, for entering data and/or commands into the electronic interactive content/media computer 70, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering data and/or information, and/or any electronic interactive content or media, which input device(s) are also connected to the CPU 70A. The electronic interactive content/media computer 70 also includes a display device 70E for displaying data and/or information to a user or operator.

The electronic interactive content/media computer 70 also includes a transmitter(s) 70F, for transmitting signals and/or data and/or information to any one or more of the central processing computer(s) 10, the provider communication device(s) 20, the payer computers(s) 30, the user or patient communication device(s) 40, the intermediary computer(s) 50, the healthcare records computer(s) 60, any other electronic interactive content/media computer(s) 70, the insurance exchange computer(s) 80, the social networking computer(s) 90, and/or the media computer(s) 95, described herein, which may be utilized in conjunction with the present invention. The electronic interactive content/media computer 70 also includes a receiver 70G, for receiving signals and/or data and/or information from any one or more of the central processing computer(s) 10, the provider communication device(s) 20, the payer computers(s) 30, the user or patient communication device(s) 40, the intermediary computer(s) 50, the healthcare records computer(s) 60, any other electronic interactive content/media computer(s) 70, the insurance exchange computer(s) 80, the social networking computer(s) 90, and/or the media computer(s) 95, described herein, which may be utilized in conjunction with the present invention.

The electronic interactive content/media computer 70 also includes a database(s) 70H. The database 70H can contain and/or be linked to any of the data and/or information described herein as being stored in the database 10H and can also contain any data and/or information regarding and/or including any electronic interactive content or media which is or can be stored in the electronic interactive content/media computer 70.

The electronic interactive content/media computer 70 also includes an output device 701 for outputting any electronic interactive content or media or for outputting any data, information, report, etc., described herein. In the preferred embodiment, the output device 701 can be a printer, a display, a transmitter, a modem, and/or any other device which can be used to output data.

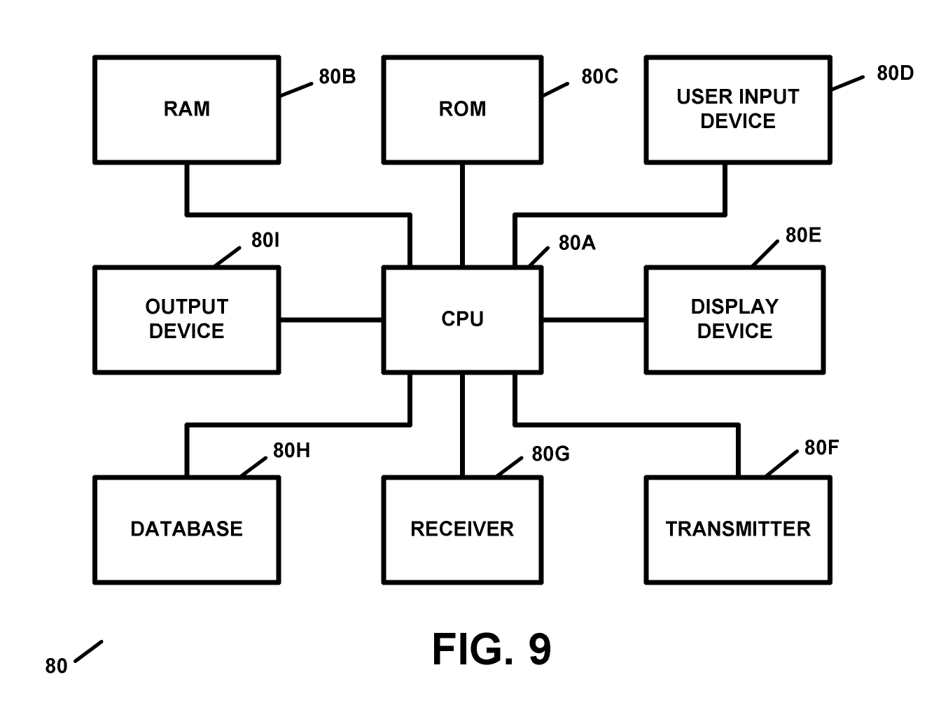

FIG. 9 illustrates the insurance exchange computer 80, in block diagram form. The insurance exchange computer 80, in the preferred embodiment, can be server computer or server computer system, a cloud computer or cloud computer system, a network computer or computer system, a personal computer or any other computer or communication device, or any cellular telephone, tablet, personal digital assistant, wireless communication device or wireless computer, or any other communication device, which is utilized to function as an insurance exchange computer 80. In a preferred embodiment, the insurance exchange computer 80 can be any computer or computer system for providing the functionality described herein as being provided by the insurance exchange computer 80.

The insurance exchange computer 80 can be used by, maintained by, or associated with any provider of any of the herein-described insurance policies, products, or services, described herein as being provided via the apparatus 100 and method of the present invention.

In the preferred embodiment, the insurance exchange computer 80 includes a central processing unit or CPU 80A, which in the preferred embodiment, is a microprocessor. The CPU 80A may also be a microcomputer, a minicomputer, a macro-computer, and/or a mainframe computer, depending upon the application.

The insurance exchange computer 80 also includes a random access memory device(s) 80B (RAM) and a read only memory device(s) 80C (ROM), each of which is connected to the CPU 80A, a user input device 80D, for entering data and/or commands into the insurance exchange computer 80, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering data and/or information which input device(s) are also connected to the CPU 80A. The insurance exchange computer 80 also includes a display device 80E for displaying data and/or information to a user or operator.

The insurance exchange computer 80 also includes a transmitter(s) 80F, for transmitting signals and/or data and/or information to any one or more of the central processing computer(s) 10, the provider communication device(s) 20, the payer communication device(s) 30, the user or patient communication device(s) 40, and the intermediary computer(s) 50, the healthcare records computer(s) 60, any other insurance exchange computer(s) 80, the social networking computer(s) 90, and/or the media computer(s) 95, described herein, which may be utilized in conjunction with the present invention. The insurance exchange computer 80 also includes a receiver 80G, for receiving signals and/or data and/or information from any one or more of the central processing computer(s) 10, the provider communication device(s) 20, the payer communication device(s) 30, the user or patient communication device(s) 40, the intermediary computer(s) 50, the healthcare records computer(s) 60, any other insurance exchange computer(s) 80, the social networking computer(s) 90, and/or the media computer(s) 95, described herein, which may be utilized in conjunction with the present invention.

The insurance exchange computer 80 also includes a database(s) 80H. The database 80H can contain and/or be linked to any of the data and/or information described herein as being stored in the database 10H and can also contain any data and/or information regarding and including any of the various insurance policies, products, or service, which can be offered for sale by the insurance exchange computer 80, as well as any data and/or information regarding insured individuals or entities, their insurance policies, products, and/or services, claims made, claims paid, and any other records typically stored or maintained by an insurance company computer, an insurance exchange computer, an electronic commerce computer or any other data and/or information for providing any of the functions or functionality described herein as being performed by the insurance exchange computer 80.

The insurance exchange computer 80 also includes an output device 801 for outputting any data, information, report, etc., described herein. In the preferred embodiment, the output device 801 can be a printer, a display, a transmitter, a modem, and/or any other device which can be used to output data.

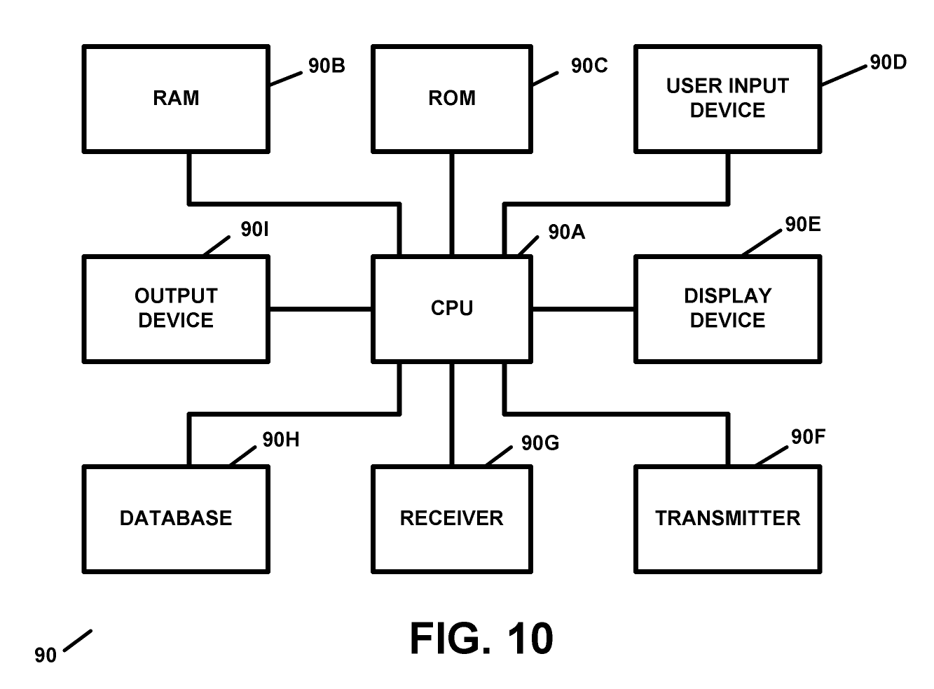

FIG. 10 illustrates the social networking computer 90, in block diagram form. The social networking computer 90, in the preferred embodiment, can be any social networking computer or computer system or any computer or computer system used by or associated with any social networking company or social networking web site, or can be any suitable server computer or server computer system, a cloud computer or cloud computer system, a network computer or computer system, a personal computer or any other computer or communication device, or any cellular telephone, tablet, personal digital assistant, wireless communication device or wireless computer, or any other communication device, which is utilized to function as a social networking computer 90. In a preferred embodiment, the social networking computer 90 can be any computer of computer system for providing the functionality described herein as being provided by the social networking computer 90 or any social networking computer or website.

The social networking computer 90 can be used by, maintained by, or associated with social networking company, web site, or other entity and can provide any of the herein-described social networking information, activities, and/or functionality, described herein as being provided via the apparatus 100 and method of the present invention and which can be provided by any social networking company, website, or other entity.

In the preferred embodiment, the social networking computer 90 includes a central processing unit or CPU 90A, which in the preferred embodiment, is a microprocessor. The CPU 90A may also be a microcomputer, a minicomputer, a macro-computer, and/or a mainframe computer, depending upon the application.

The social networking computer 90 also includes a random access memory device(s) 90B (RAM) and a read only memory device(s) 90C (ROM), each of which is connected to the CPU 90A, a user input device 90D, for entering data and/or commands into the social networking computer 90, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering data and/or information which input device(s) are also connected to the CPU 90A. The social networking computer 90 also includes a display device 90E for displaying data and/or information to a user or operator.

The social networking computer 90 also includes a transmitter(s) 90F, for transmitting signals and/or data and/or information to any one or more of the central processing computer(s) 10, the provider communication device(s) 20, the payer communication device(s) 30, the user or patient communication device(s) 40, and the intermediary computer(s) 50, the healthcare records computer(s) 60, the insurance exchange computer(s) 80, any other social networking computer(s) 90, and/or the media computer(s) 95, described herein, which may be utilized in conjunction with the present invention. The social networking computer 90 also includes a receiver 90G, for receiving signals and/or data and/or information from any one or more of the central processing computer(s) 10, the provider communication device(s) 20, the payer communication device(s) 30, the user or patient communication device(s) 40 the intermediary computer(s) 50, the healthcare records computer(s) 60, the insurance exchange computer(s) 80, any other social networking computer(s) 90, and/or the media computer(s) 95, described herein, which may be utilized in conjunction with the present invention.

The social networking computer 90 also includes a database(s) 90H. The database 90H can contain and/or be linked to any of the data and/or information described herein as being stored in the database 10H and can also contain any data and/or information typically found in or utilized by a social networking computer or any members of the social network serviced thereby as well as any data and/or information typically utilized in providing or performing the functionality and/or services provided or offered by the social network associated with the social networking computer 90.

The social networking computer 90 also includes an output device 901 for outputting any data, information, report, etc., described herein. In the preferred embodiment, the output device 901 can be a printer, a display, a transmitter, a modem, and/or any other device which can be used to output data.

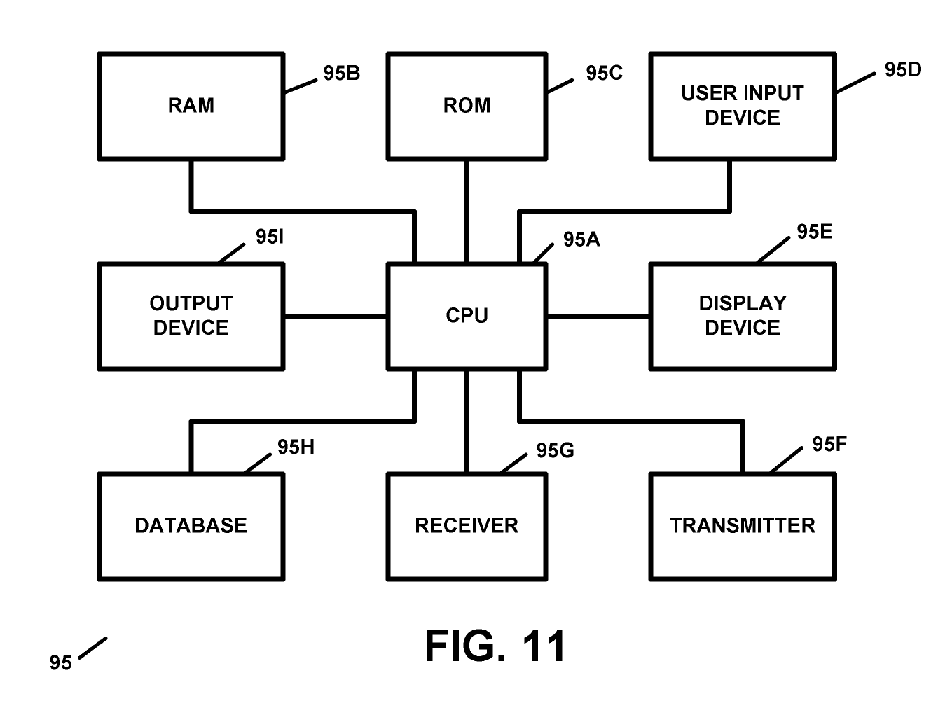

FIG. 11 illustrates the media computer 95, in block diagram form. The media computer 95, in the preferred embodiment, can be any computer or computer system used by or associated with any news information source, individual, company, or entity, any external information source, individual, company, or entity, any advertiser or advertisement source, individual, company or entity, or any marketing materials source, individual, company, or entity.

The media computer 95 can be any computer or computer system which can provide, to any of the herein-described users, individuals, patients, providers, payers, intermediaries, third parties, and/or entities who or which utilized the apparatus 100 and method of the present invention, any news, information, advertisements, or marketing materials, of any kind, nature, or type, which can include, but which is not limited to, any healthcare or healthcare-related news, information, advertisements, or marketing materials, any fitness or fitness-related news, information, advertisements, or marketing materials, any wellness or wellness-related news, information, advertisements, or marketing materials, as well as any non-healthcare or non-healthcare-related news, information, advertisements, or marketing materials. The media computer 95 provides news, information, advertisements, or marketing materials, via the apparatus 100 and method of the present invention. In a preferred embodiment, the media computer 95 can be any computer or computer system for providing the functionality described herein as being provided by the media computer 95.

The media computer 95 can be used by, maintained by, or associated with a media company or entity, a news service, information source or service, an advertiser, an advertisement company, agency, or entity, a marketer, a marketing company or entity, or any individual providing a media, news, information, advertisements, or marketing materials of or on behalf of themselves or another, The media computer 95 can provide any of the herein-described media, news, information, advertisements, or marketing materials, and/or any other information and/or can provide any of the functionality described herein as being provided via the apparatus 100 and method of the present invention.

In the preferred embodiment, the media computer 95 includes a central processing unit or CPU 95A, which in the preferred embodiment, is a microprocessor. The CPU 95A may also be a microcomputer, a minicomputer, a macro-computer, and/or a mainframe computer, depending upon the application.

The media computer 95 also includes a random access memory device(s) 95B (RAM) and a read only memory device(s) 95C (ROM), each of which is connected to the CPU 95A, a user input device 95D, for entering data and/or commands into the media computer 95, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering data and/or information which input device(s) are also connected to the CPU 95A. The media computer 95 also includes a display device 95E for displaying data and/or information to a user or operator.

The media computer 95 also includes a transmitter(s) 95F, for transmitting signals and/or data and/or information to any one or more of the central processing computer(s) 10, the provider communication device(s) 20, the payer communication device(s) 30, the user or patient communication device(s) 40, and the intermediary computer(s) 50, the healthcare records computer(s) 60, the insurance exchange computer(s) 80, the social networking computer(s) 90, and/or any other media computer(s) 95, described herein, which may be utilized in conjunction with the present invention. The media computer 95 also includes a receiver 95G, for receiving signals and/or data and/or information from any one or more of the central processing computer(s) 10, the provider communication device(s) 20, the payer communication device(s) 30, the user or patient communication device(s) 40 the intermediary computer(s) 50, the healthcare records computer(s) 60, the insurance exchange computer(s) 80, the social networking computer(s) 90, and/or any other media computer(s) 95, described herein, which may be utilized in conjunction with the present invention.

The media computer 95 also includes a database(s) 95H. The database 95H can contain and/or be linked to any of the data and/or information described herein as being stored in the database 10H and can also contain any data and/or information typically found in or utilized by a media computer as well as any of the respective media, news, information, advertisements, or marketing materials, and/or any data and/or information typically utilized in providing or performing the functionality and/or services provided or offered by the media computer 95.

The media computer 95 also includes an output device 95I for outputting any data, information, report, etc., described herein. In the preferred embodiment, the output device 95I can be a printer, a display, a transmitter, a modem, and/or any other device which can be used to output data.

The apparatus and method of the present invention can be utilized in numerous preferred embodiments in order to provide a vast array of healthcare and healthcare-related services for any one or more of the various parties described herein. While certain of the preferred embodiments may be described with regards to utilization by a particular party, it is important to note that any individual, patient, user, provider, payer, and/or intermediary or third party, may utilize the present invention in the same, similar and/or analogous manner. For example, a preferred embodiment for determining and/or ascertaining a medical diagnosis can be described as being utilized by a treating physician as well as be utilized by a provider to verify and/or check a diagnosis as well as by a patient or other user or individual in order to perform a self-diagnosis or double check a doctors diagnosis. In the same manner, any other preferred embodiment and/or other uses of the present invention can be utilized by any of the parties described herein.

In a preferred embodiment, the apparatus 100 of the present invention can be utilized to create and maintain a comprehensive and/or a centralized electronic healthcare record system. It is understood that different providers and insurers or payers might utilize or employ any number or types or kinds of electronic healthcare records, electronic medical records, electronic dental records, electronic pharmacy records, and/or electronic behavioral healthcare records, each of which may be located in or on different computers or located at different physical locations, and that any individual, patient, or user, may have a healthcare or healthcare-related record, a healthcare or healthcare-related file, or a healthcare or healthcare-related history, on any one or more of these separate electronic healthcare records, electronic medical records, electronic dental records, electronic pharmacy records, and/or electronic behavioral healthcare records. It is also understood that each individual or patient may also have a personal health record stored or located on his or her user or patient communication device 40.

In a preferred embodiment, any one or more of the healthcare records computers 60, can be associated with a different electronic healthcare record, electronic medical record, electronic dental record, electronic pharmacy record, and/or electronic behavioral healthcare record, or any number or combination of electronic healthcare records, electronic medical records, electronic dental records, electronic pharmacy records, and/or electronic behavioral healthcare records.

In a preferred embodiment, the database 10H can, for each individual or patient who utilize the apparatus 100 and method of the present invention, contain any number of electronic healthcare records, electronic medical records, electronic dental records, electronic pharmacy records, and/or electronic behavioral healthcare records, containing data and/or information regarding the individual or patient, and/or the database 10H can contain any number of links or hyperlinks to any number of electronic healthcare records, electronic medical records, electronic dental records, electronic pharmacy records, and/or electronic behavioral healthcare records, which are located on healthcare records computers 60, or on any other computers, or computer systems, which are separate and apart from the central processing computer 10 and which also contain data and/or information for or regarding the individual or patient.

In an preferred embodiment, and in instances where an individual's or patient's healthcare-related records, healthcare or healthcare-related files, or healthcare or healthcare-related histories, are stored and/or located in different or separate electronic healthcare records, electronic medical records, electronic dental records, electronic pharmacy records, and/or electronic behavioral healthcare records, or stored or located on different or separate computers or computer systems, including, but not limited to, the central processing computer 10, healthcare records computers 60, or any other computers or computer systems, the apparatus 100, and in particular, the database 10H or the central processing computer can also contain an master individual or patient index containing, for each individual or patient, the individual's or patient's name, address, social security number or other uniquely assigned identification number or identifying data or information, mother's maiden name, name of spouse or partner, name(s) of child, children or others for who the individual or patient is responsible for providing care legally or otherwise, each electronic healthcare records, electronic medical records, electronic dental records, electronic pharmacy records, and/or electronic behavioral healthcare records, containing data and/or information regarding the individual or patient, or in or on which the individual or patient has a record, file, history, or entry, and/or a link(s) or hyperlink(s) to each electronic healthcare records, electronic medical records, electronic dental records, electronic pharmacy records, and/or electronic behavioral healthcare records, containing data and/or information regarding the individual or patient, or in or on which the individual or patient has a record, file, history, or entry.

The apparatus and method of the present invention can be utilized in order to process information and/or to provide information regarding a diagnosis or possible diagnoses for or regarding an individual or a patient, to process and/or to provide diagnostic information based on a symptom or symptoms, an observation or observations, an examination finding or examination findings, any data and/or information obtained from a procedure performed on or with an individual or a patient or obtained from or during an examination, a test, a procedure, or an analysis, or any data and/or information obtained from or with any electronic interactive activity or electronic interactive activities described herein.

The apparatus and method of the present invention can also be utilized in order to process information and/or to provide information regarding a treatment, a prescribed treatment, or possible or available treatments, as well as any treatment plan or treatment plans associated with same, for an individual or a patient, which can include processing and/or providing data and/or information for or associated with identifying, prescribing, providing, administering, monitoring, recoding, and/or processing any data and/or information obtained from or with, any electronic interactive activity or electronic interactive activities which can be utilized in a treatment or an treatment plan for the individual or the patient.

In another preferred embodiment, the apparatus 100 of the present invention can also provide for automatic claim form generation and submission or request for payment form generation and submission via the central processing computer 10 once a final diagnosis and/or a treatment has been prescribed by a healthcare provider, and/or upon the occurrence of an examination and/or the administration of a treatment, or the performance of a procedure.

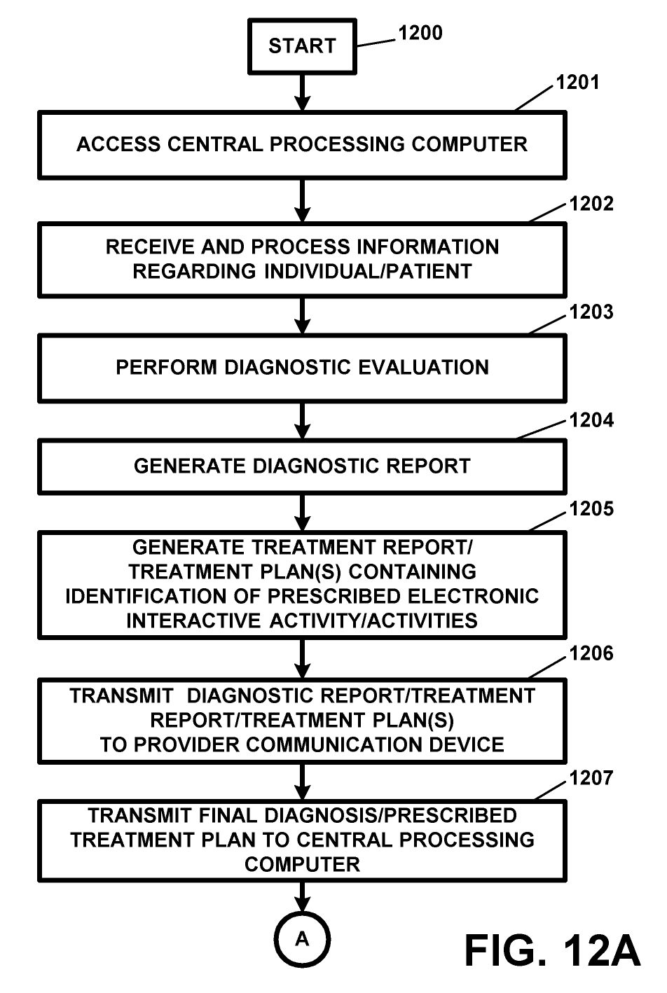

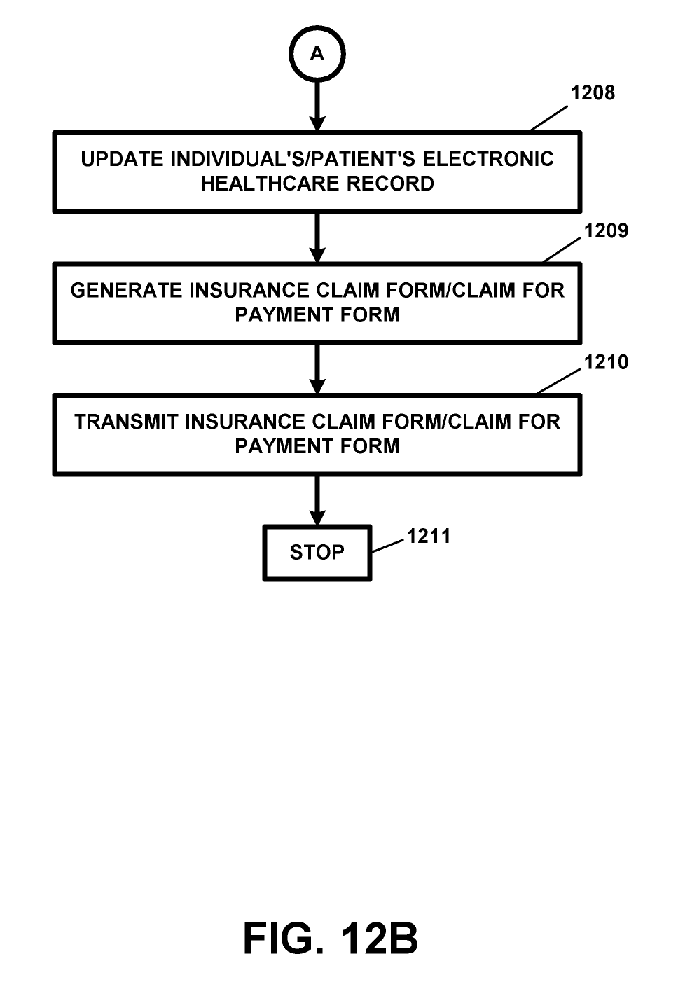

FIGS. 12A and 12B illustrate a preferred embodiment method of utilizing the apparatus 100 of the present invention, in flow diagram form. While the method of FIGS. 12A and 12B is described in the context of a medical doctor performing a diagnosis of, and/or prescribing a treatment or a treatment plan for, a medical condition, the method of FIGS. 12A and 12B can be utilized in a same, a similar, and/or an analogous, manner by any healthcare providers or providers described herein, including, but not limited to, physicians, medical doctors having any specialty, surgeons, dentists, orthodontists, periodontists, endodontists, oral surgeons, osteopaths, psychologists, psychiatrists, optometrists, podiatrists, osteopaths, chiropractors, pharmacists, therapists, physical therapists, respiratory therapists, nurses, healthcare aids, nurse practitioners, physician's assistants, nutritionists, and/or any other person, individual, and/or professional, who can provide healthcare, healthcare-related, and/or wellness and/or wellness-related, services and/or products, or insurance companies, healthcare insurance companies, disability insurance companies, property or casualty insurance companies, health maintenance organizations, healthcare providers, and/or any other payer and/or provider of healthcare services and/or products, healthcare claims processing centers, healthcare insurance brokers and/or agents, and/or any other third party and/or intermediary who or which acts on behalf of another and/or who or which assists in the providing of healthcare and/or related services, and/or any other healthcare provider or healthcare professional described herein (hereinafter also referred to in this preferred embodiment as a “provider” or “user”).

The method of FIGS. 12A and 12B may also be utilized by any user, individual, patient, caregiver, provider, insurer, payer, and/or intermediary, in order to ascertain a diagnosis, a treatment, or a treatment plan and/or in order to check on, verify, and/or ascertain, the correctness of a diagnosis and/or to formulate, plan, check on, verify, and/or ascertain, the correctness of a diagnosis, a treatment, or a treatment plan. With reference to FIGS. 12A and 12B, the operation of the apparatus 100 commences at step 1200. At step 1201, the provider or other user can access the central processing computer via the provider communication device 20. At step 1201, information regarding the individual or the patient, which can include, but which is not limited to, the individual's or patient's symptoms, if any, and/or any examination findings or observations are or can be obtained from the individual or the patient, can be entered into the provider communication device 20, and can then be transmitted from the provider communication device 20 to the central processing computer 10.

The central processing computer 10, or the CPU 10A, can, at step 1202, receive and/or process the information regarding the individual or the patient which can include the individual's or the patient's symptoms, if any, and/or the examination findings or observations, and can process same using or in conjunction with any other data and/or information contained in the individual's or the patient's electronic healthcare record or electronic healthcare file which is stored in the database 10H of the central processing computer 10 and/or with or using any healthcare or medical information, theories, principles, criteria, and/or any other healthcare information or medical information needed to make a diagnosis. In another preferred embodiment, if the individual or the patient had previously engaged in, performed, or participated in, any electronic interactive activity or any electronic interactive activities, then any data and/or information obtained from or regarding same, can also be utilized by the central processing computer 10 in order to make or to arrive at a diagnosis for the individual or the patient.

At step 1203, the central processing computer 10, or the CPU 10A, can perform a diagnostic evaluation of the individual or the patient. In a preferred embodiment, at step 1203, the central processing computer 10, or the CPU 10A, can perform a diagnostic evaluation of the individual or the patient by using any of the data and/or information described herein as being processed by the central processing computer 10 at step 1202.

At step 1204, the central processing computer 10, or the CPU 10A, can generate a diagnostic report which can include a diagnosis of the individual's or the patient's condition. The diagnostic report, which is generated at step 1204, can include a single diagnosis and/or a list of possible diagnoses, along with their respective probabilities of being accurate, which may pertain to the individual's or the patient's condition. In a preferred embodiment, the diagnostic report can also contain information identifying an electronic interactive activity or any number of electronic interactive activities which might identified by the central processing computer 10 or the CPU 10A as being deemed to be useful, desired, or required, in conducting further a diagnostic evaluation or analysis of the individual or the patient. In a preferred embodiment, at step 1204, the central processing computer 10, or the CPU 10A, can utilize data and/or information contained in the database 10H in order to identify any electronic interactive activity or any electronic interactive activities which can be utilized in performing a diagnostic evaluation of the individual or the patient.

In a preferred embodiment, the electronic interactive activity or electronic interactive activities identified in the diagnostic report can be a video game, an audio game, a video and audio game, an electronic or digital exercise, an electronic or digital puzzle or activity, an electronic or digital diagnostic activity or tool, an electronic or digital treatment, treating planning, or therapy, activity or tool, an electronic or digital mental health or behavioral health activity or tool, an electronic or digital physical or physical therapy activity or tool, an electronic or digital training activity or tool, an electronic or digital rehabilitation activity or tool, an electronic or digital mental health or behavioral health training or re-training activity or tool, an electronic or digital physical training or re-training activity or tool, a survey, a questionnaire, or any other game, exercise, activity, information gathering event or occurrence, or any other content or media which can be utilized, or any number of same, or any combination or same, which is prescribed for performing a diagnostic evaluation or analysis of the individual or the patient.

In a preferred embodiment, for example, the central processing computer 10, or the CPU 10A, can process data and/or information regarding any symptoms, if any, and/or the examination findings or observations, in conjunction with any data and/or information stored in the database 10H which relates or correlates any electronic interactive activity or any electronic interactive activities to any diagnosis or diagnostic evaluation for or regarding any symptoms, diseases, conditions, illnesses, syndromes, sicknesses, and/or states of dysfunction, relating to the symptoms, if any, and/or the examination findings or observations in order to identify any electronic interactive activity or any electronic interactive activities which can utilized in performing a further diagnostic evaluation of the individual or the patient.

At step 1205, the central processing computer 10 can then generate a treatment report which can or which will outline and/or prescribe a treatment(s) for the single diagnosis and/or a treatment(s) for each of the diagnoses in list of possible diagnoses. In a preferred embodiment, the central processing computer 10, at step 1205, in generating the treatment report, can identify an electronic interactive activity or can identify any number of electronic interactive activities which can or which may be appropriate or suitable in treating the individual or the patient, in performing further diagnostic activities, or in providing therapy, rehabilitation, training, or re-training, for the individual's of the patient's diagnosis and/or treatment regimen.

In a preferred embodiment, the treatment report can also contain information identifying an electronic interactive activity or any number of electronic interactive activities which might identified by the central processing computer 10 or the CPU 10A as being deemed to be useful, desired, or required, in treating the diagnosis or each of the possible diagnoses identified in the diagnostic report. In a preferred embodiment, at step 1205, the central processing computer 10, or the CPU 10A, can utilize data and/or information contained in the database 10H in order to identify any electronic interactive activity or any electronic interactive activities which can be utilized in treating the diagnosis or each of the possible diagnoses identified in the diagnostic report. In a preferred embodiment, for example, the central processing computer 10, or the CPU 10A, can process data and/or information regarding any identified diagnosis in conjunction with any data and/or information stored in the database 10H which relates or correlates any electronic interactive activity or any electronic interactive activities to the identified diagnosis in order to identify any electronic interactive activity or electronic interactive activities which can be utilized in treating the identified diagnosis or in treating each identified diagnosis in the list of possible diagnoses.

In a preferred embodiment, the electronic interactive activity or electronic interactive activities identified in the treatment report can be a video game, an audio game, a video and audio game, an electronic or digital exercise, an electronic or digital puzzle or activity, an electronic or digital diagnostic activity or tool, an electronic or digital treatment, treating planning, or therapy, activity or tool, an electronic or digital mental health or behavioral health activity or tool, an electronic or digital physical or physical therapy activity or tool, an electronic or digital training activity or tool, an electronic or digital rehabilitation activity or tool, an electronic or digital mental health or behavioral health training or re-training activity or tool, an electronic or digital physical training or re-training activity or tool, a survey, a questionnaire, or any other game, exercise, activity, information gathering event or occurrence, or any other content or media which can be utilized, or any number of same, or any combination or same, which is prescribed for treating the identified diagnosis or in treating each identified diagnosis in the list of possible diagnoses.

At step 1205, the central processing computer 10 can also generate a treatment plan for the diagnosis or for each diagnosis in the list of diagnoses. In a preferred embodiment, the central processing computer 10, at step 1205, in generating the treatment plan, can identify an electronic interactive activity or can identify any number of electronic interactive activities which can or which may be appropriate or suitable in treating the individual or the patient, in performing further diagnostic activities, or in providing therapy, rehabilitation, training, or re-training, for the individual's of the patient's diagnosis and/or treatment regimen. In a preferred embodiment, the central processing computer 10, or the CPU 10A, when generating the treatment report and/or the treatment plan, or each treatment report or each treatment plan, can also process any information regarding or contained in same in conjunction with any information regarding any physical limitations, mental health limitations or behavioral limitations, emotional limitations, or any other limitations, known to be associated with or regarding the individual or the patient, and/or the central processing computer 10 can also process any information regarding or contained in same in conjunction with any information regarding any possible drug interactions and/or treatment interactions, and/or individual or patient allergies, as well as any treatments, medications, therapies, or tests, or any other activities, which might prove to be harmful to the individual or the patient.

In a preferred embodiment, the treatment plan can also contain information identifying an electronic interactive activity or any number of electronic interactive activities which might identified by the central processing computer 10 or the CPU 10A as being deemed to be useful, desired, or required, in treating the diagnosis or each of the possible diagnoses identified in the diagnostic report. In a preferred embodiment, at step 1205, the central processing computer 10, or the CPU 10A, can utilize data and/or information contained in the database 10H in order to identify any electronic interactive activity or any electronic interactive activities which can be utilized in treating the diagnosis or each of the possible diagnoses identified in the diagnostic report. In a preferred embodiment, for example, the central processing computer 10, or the CPU 10A, can process data and/or information regarding any identified diagnosis in conjunction with any data and/or information stored in the database 10H which relates or correlates any electronic interactive activity or any electronic interactive activities to the identified diagnosis in order to identify any electronic interactive activity or electronic interactive activities which can be utilized in treating the identified diagnosis or in treating each identified diagnosis in the list of possible diagnoses.

In a preferred embodiment, the electronic interactive activity or electronic interactive activities identified in the treatment plan can be a video game, an audio game, a video and audio game, an electronic or digital exercise, an electronic or digital puzzle or activity, an electronic or digital diagnostic activity or tool, an electronic or digital treatment, treating planning, or therapy, activity or tool, an electronic or digital mental health or behavioral health activity or tool, an electronic or digital physical or physical therapy activity or tool, an electronic or digital training activity or tool, an electronic or digital rehabilitation activity or tool, an electronic or digital mental health or behavioral health training or re-training activity or tool, an electronic or digital physical training or re-training activity or tool, a survey, a questionnaire, or any other game, exercise, activity, information gathering event or occurrence, or any other content or media which can be utilized, or any number of same, or any combination or same, which is prescribed for treating the identified diagnosis or in treating each identified diagnosis in the list of possible diagnoses.

In a preferred embodiment, the treatment report and/or the treatment plan can include information regarding a plan of treatment, including, but not limited to, any and/or all electronic interactive activities in which the individual or the patient should engage in, perform, or participate in, as well as any necessary, recommended, or suggested, doctor visits, office visits, provider visits, hospital stays or stays in healthcare facilities or rehabilitative facilities, tests, testing, procedures, treatments, surgeries, consultations, therapy sessions, and/or rehabilitation sessions, and/or or any medications, drugs, alternative medicine treatments or products, goods, or services, and/or any other goods, products, or services, and/or any other healthcare or other services or goods, which may be prescribed, needed, or deemed desirable, for that treatment plan, which are or which might be called for by a respective treatment report or treatment plan.

In a preferred embodiment, as well as in any and/or all of the embodiments described herein, any of the herein-described providers, suppliers, or vendors, of any of the herein-described electronic interactive activities, can access the central processing computer 10 at any time using his, her, or its, respective provider communication device 20 or intermediary communication device 50, and can transmit or upload, to the central processing computer 10 and/or to the electronic interactive content/media computer 70, his, her, or its, respective electronic interactive activity or electronic interactive activities.

In another preferred embodiment, the treatment report and/or the treatment plan, or each treatment report or treatment plan corresponding to each possible diagnosis, can also contain or include information regarding, and/or a link(s) or a hyperlink(s) to, any of the electronic interactive activities identified in the respective treatment report or treatment plan, along with any information, or any link or hyperlink to any information for or regarding each electronic interactive activity including, but not limited to, instructions for using same, purpose of same in the course of treatment, and/or any other pertinent information regarding same. In another preferred embodiment, the treatment report and/or the treatment plan, or each treatment report or treatment plan corresponding to each possible diagnosis, can also contain or include information regarding the prescribed used of any electronic interactive activity or electronic interactive activities, prescribed number of times for using same, prescribed usage frequency, and/or any other pertinent information regarding how a respective electronic interactive activity is to be utilized or employed in the treatment plan or the treatment process. In another preferred embodiment, the treatment report and/or the treatment plan, or each treatment report or treatment plan corresponding to each possible diagnosis, can also contain or include information regarding, and/or a link(s) or a hyperlink(s) to, any of the providers, suppliers, or vendors, of any of the prescribed electronic interactive activities or to any computers, communication devices, or websites, of or associated with same.

In another preferred embodiment, the treatment report and/or the treatment plan, or each treatment report or treatment plan corresponding to each possible diagnosis, can also contain or include information regarding, and/or a link(s) or a hyperlink(s) to, a provider communication device 20 of each of the individual's or the patient's healthcare providers or doctors and/or to healthcare providers or doctors identified as being able to treat the individual or the patient, so that the individual or the patient, or a caregiver, can contact or communicate with a respective healthcare provider or doctor, or so that the individual or the patient, or a caregiver, can schedule an appointment with the healthcare provider or doctor via the central processing computer 10 or otherwise, or so that the individual or the patient can utilize the information or the link(s) or hyperlink(s) for any other appropriate reason.

In another preferred embodiment, the treatment report and/or the treatment plan, or each treatment report or treatment plan corresponding to each possible diagnosis, can also contain or include information regarding, and/or a link(s) or a hyperlink(s), to an insurer communication device 30 of each insurer or payer of the individual or patient so that the individual or the patient, or a caregiver, can contact or communicate with a respective healthcare insurer or payer, so that the individual or the patient can submit an insurance claim to the insurer or payer via the central processing computer 10 or otherwise, so that the individual or the patient can submit a pre-authorization request to the insurer or payer, or so that the individual or the patient can utilize the information or the link(s) or hyperlink(s) for any other appropriate reason.

In another preferred embodiment, the treatment report and/or the treatment plan, or each treatment report or treatment plan corresponding to each possible diagnosis, can also contain or include information regarding, and/or a link(s) or a hyperlink(s), to the central processing computer 10 and/or to the healthcare records computer 60 so that the individual or the patient can access his or her electronic healthcare record(s) or any portion of same. In another preferred embodiment, the treatment report and/or the treatment plan, or each treatment report or treatment plan corresponding to each possible diagnosis, can also contain or include information regarding, and/or a link(s) or a hyperlink(s), to the central processing computer 10 and/or to the electronic interactive content/media computer 70 of or associated with any provider, supplier, or vendor, of any prescribed electronic interactive activity prescribed therein, so that the individual or the patient. In another preferred embodiment, the treatment report and/or the treatment plan, can also contain or include information regarding, and/or a link(s) or a hyperlink(s), to the insurance exchange computer 80 so that the individual or the patient can access the insurance exchange computer 80 in order file a healthcare insurance claim, check on the status of a healthcare insurance claim, or purchase a healthcare or other insurance policy.

In another preferred embodiment, the treatment report and/or the treatment plan, or each treatment report or treatment plan corresponding to each possible diagnosis, can also contain or include information regarding, and/or a link(s) or a hyperlink(s), to a social networking computer 90 and/or to any social networking web site or web pages, and/or to or for any social networking members, support groups, social networking support group members, on-line seminars, social networking lectures, classes, or seminars, social networking sponsored lectures, classes, or seminars, social networking discussions, question and answer sessions, or informational or other forums, or any other social networking or social networking sponsored activities or events, forums, chat room discussions, or any other information providing sources, with which or with whom the individual or the patient may engage. The providing of the social networking information to the individual or the patient can serve to allow the individual or the patient to learn more about a diagnosis, a treatment, or a treatment plan, or to interact with others who have been diagnosed with the same or a similar ailment, illness, sickness, or condition, or to interact with others who may be undergoing the same or a similar treatment, or who may be following a same or a similar treatment plan.

In another preferred embodiment, the treatment report or the treatment plan can also contain or include information prescribing that the individual or the patient engage in an electronic interactive activity or electronic interactive activities along with other individuals or patients or in a group electronic interactive activity or in group electronic interactive activities.

In another preferred embodiment, the treatment report and/or the treatment plan, or each treatment report or treatment plan corresponding to each possible diagnosis, can also contain or include information regarding, and/or a link(s) or a hyperlink(s), to a media computer 95 so that the individual or the patient can access news articles or other information regarding his or her diagnosis and/or treatment plan and/or regarding any of the herein-described electronic interactive activities which may be provided by the apparatus 100 and method of the present invention.

In a preferred embodiment, any of the herein-described diagnostic reports, treatment reports, or treatment plans, can also include or can contain data and/or information stored in the individual's or the patient's electronic healthcare record, a link(s) or a hyperlink(s) to data and/or information stored in the individual's or the patient's electronic healthcare record, any notes, comments, or messages, entered by the individual or the patient, or by a caregiver, or by a provider, into the individual's or the patient's electronic healthcare record, and/or any link(s) or hyperlink(s) to any notes, comments, or messages, entered by the individual or the patient, or by a caregiver, or by a provider, into the individual's or the patient's electronic healthcare record.

At step 1206, the central processing computer 10 will transmit the diagnostic report and/or the treatment report and/or the treatment plan(s) for the diagnosis or for each of the possible diagnoses to the provider's provider communication device 20, at which point the provider, or the healthcare provider or medical doctor, can obtain the diagnosis or list of possible diagnoses and the corresponding treatment report(s) or treatment plan(s) for same. The provider, or the healthcare provider or medical doctor, can then review the diagnostic report and/or the treatment report(s) and/or the treatment plan(s), and choose, select, or decide upon, a final diagnosis and/or a prescribed treatment plan to administer to the individual or the patient.

At step 1207, the provider, or the healthcare provider or medical doctor, will transmit the final diagnosis and the prescribed treatment or prescribed treatment plan, including the prescribed treatment and/or the treatment plan corresponding to same, to the central processing computer 10. In another preferred embodiment, at step 1207, the provider, or the healthcare provider or medical doctor, or any other provider using the apparatus 100, can also generate and/or transmit a prescription or an electronic prescription for an electronic interactive activity or for any number of electronic interactive activities, a medication, a medicine, or a drug, or for a procedure, a test, an analysis, an analysis work-up, blood work, a treatment, or a therapy, or can generate and/or transmit a referral to another provider, for submission or transmission to the respective pharmacy or other provider, and for storage in the individual's, the patient's electronic healthcare record or electronic healthcare file.

In another preferred embodiment, the final diagnosis, along with the prescribed treatment plan, and the diagnostic report, the treatment report, and/or the treatment plan, generated for and corresponding to same can also be transmitted by the central processing computer 10, at step 1207, to the provider communication device 20 associated with or used by the provider for the provider's records, to a provider communication device 20 associated with or used by another provider of the individual or the patient, to a payer or insurer communication device 30 associated with or used by the insurer or payer of the individual or the patient, to the user communication device 40 associated with or used by the individual or the patient, or a caregiver, or to an intermediary communication device 50 associated with or used by a third party or an intermediary.

In a preferred embodiment, the central processing computer 10 can transmit the final diagnosis along with the prescribed treatment plan, and the diagnostic report, the treatment report, and/or the treatment plan, generated for and corresponding to same to a respective provider communication device 20, to a respective insurer or payer communication device 30, to a respective user communication device 40, or to a respective intermediary communication device 50, either directly, such as via any suitable electronic or other transmission, or indirectly, such as via an e-mail server in the case when the final diagnosis and/or the prescribed treatment plan, and the diagnostic report, the treatment report, and/or the treatment plan, generated for and corresponding to same, can be, can be included in, or can be attached to, an e-mail message, a text message, an SMS message, or any other electronic communication or transmission.

In a preferred embodiment, the final diagnosis along with the prescribed treatment plan, and the diagnostic report, the treatment report, and/or the treatment plan, generated for and corresponding to same, or a respective copy of same can be data-stamped and/or time-stamped.

In a preferred embodiment, the final diagnosis along with the prescribed treatment plan, and the diagnostic report, the treatment report, and/or the treatment plan, generated for and corresponding to same, or a respective copy of same, can also be stored by and/or in the database 20H of the respective provider communication device(s) 20, in the database 30H of respective insurer or payer communication device(s) 30, in the database 40H of the respective user communication device(s) 40, in the database 50H of the respective intermediary communication device(s) 50, in the database 60H the healthcare records computer 60, in the database 70H of the electronic interactive content/media computer 70, and/or in the database 80H of the insurance exchange computer 80.

In a preferred embodiment, the final diagnosis along with the prescribed treatment plan, and the diagnostic report, the treatment report, and/or the treatment plan, generated for and corresponding to same or a respective copy of same, can also be transmitted to and/or stored by or in the individual's or the patient's electronic healthcare record in the database 10H of the central processing computer 10, in the database 20H in the provider communication device 20, in the database 30H of the payer communication device 30, in the database 40H of the user communication device 40, or in the individual's, the patient's, or the caregiver's, electronic healthcare record in the database 60H of the healthcare records computer 60, in the database 70H of the electronic interactive content/media computer 70, or in the database 80H of the insurance exchange computer 80.

In another preferred embodiment, the final diagnosis along with the prescribed treatment plan, and the diagnostic report, the treatment report, and/or the treatment plan, generated for and corresponding to same, can also contain individual-specific or patient-specific educational information or individual-specific or patient-specific instructional information, or a link(s) or a hyperlink(s) to same, regarding the diagnosis, the treatment, or the treatment plan, or information relating thereto, so as to provide educational information or instructional information to the individual or the patient. The individual or the patient, or a caregiver, can thereafter access or obtain the educational information or instructional information via the apparatus 100 and method of the apparatus 100 of the present invention. In this manner, the apparatus 100 and method of the present invention can provide individual-specific or patient-specific educational information or individual-specific or patient-specific instructional information regarding the diagnosis, the treatment, or the treatment plan.

In another preferred embodiment, the apparatus 100 or the central processing computer 10 can be programmed to identify the individual-specific or patient-specific educational information or individual-specific or patient-specific instructional information regarding the diagnosis, the treatment, or the treatment plan, and/or any information, link(s), or hyperlink(s) relating thereto or pertaining thereto.

At step 1208, the central processing computer 10, or the CPU 10A, will then update the individual's or the patient's electronic healthcare record in the database 10H so as to include all of the data and information described as being processed and/or generated by the central processing computer 10, or the CPU 10A, including, but not limited to the individual's or the patient's symptoms or examination findings or any observations, the final diagnosis, the prescribed treatment plan, the diagnostic report(s), the treatment report(s), and/or the treatment plan(s) described as being generated herein, and/or any data and/or information contained in or regarding the final diagnosis along with the prescribed treatment plan, and the diagnostic report(s), the treatment report(s), and/or the treatment plan(s), generated for and/or corresponding to same.

Thereafter, at step 1209, the central processing computer 10, or the CPU 10A, will generate an insurance claim form or a request for payment form (hereinafter “claim form”) which can meet the formal claim submission requirements of the individual's or the patient's insurer, insurance company, or payer. In a preferred embodiment, the claim form can include or can contain any data and/or information which is stored in individual's or the patient's electronic healthcare record, a link(s) or a hyperlink(s) to the individual's or the patient's electronic healthcare record, a link(s) or a hyperlink(s) to any data and/or information stored in the individual's or the patient's electronic healthcare record, any notes, comments, or messages, entered by the individual, the patient, or a caregiver, or a provider, into the individual's or the patient's electronic healthcare record, and/or any link(s) or hyperlink(s) to any notes, comments, or messages, entered by the individual, the patient, or a caregiver, or a provider, into the individual's or the patient's electronic healthcare record. In this regard, in a preferred embodiment, any data and/or information contained or included in the individual's or the patient's electronic healthcare record can be readily available to and/or can be readily accessible to or by any insurer or payer, or any employee or agent of same, or by any other user, person, or entity, who or which reviews the claim form or is to review the claim form.

In a preferred embodiment, the claim form can also contain or include, or contain a link(s) or a hyperlink(s) to the final diagnosis, the prescribed treatment plan, the diagnostic report(s), the treatment report(s), and/or the treatment plan(s) described as being generated herein, and/or any data and/or information contained in or regarding the final diagnosis along with the prescribed treatment plan, and the diagnostic report(s), the treatment report(s), and/or the treatment plan(s), generated for and/or corresponding to same. In another preferred embodiment, the claim form can also contain or include, or contain a link(s) or a hyperlink(s) to any electronic interactive activity or any electronic interactive activities identified or prescribed in any of the herein-described diagnostic report(s), treatment report(s), and/or treatment plan(s). It is important to note that, in a preferred embodiment, if needed, any diagnostic report described herein as being generated by the central processing computer 10 or the CPU 10A, or by the apparatus, can also prescribe use of, and/or identify, any electronic interactive activity or any electronic interactive activities which might be identified as being useful in performing a further diagnostic evaluation or analysis of the individual or the patient.

At step 1210, the claim form can be automatically transmitted or submitted by or from the central processing computer 10 to the respective insurer or payer communication device 30 of the respective insurer or payer of the individual or the patient. In another preferred embodiment, the claim form, or a copy of same, can also be transmitted by the central processing computer 10, at step 1210, to the provider, to the individual or the patient, or to a third party or intermediary, by transmitting the claim form, or a copy of same, and/or any of the herein-described included or attached information, to a respective provider communication device 20 associated with or used by the provider or any other provider of the individual or patient, to a respective user communication device 40 associated with or used by the individual or the patient, or to a caregiver for the individual or the patient, or to a respective intermediary communication device 50 associated with or used by an intermediary or third party, either directly, such as via any suitable electronic or other transmission, or indirectly, such as via an e-mail server in the case when the copy of the claim form is included in, or is attached to, an e-mail message, a text message, as SMS message, or any other electronic transmission.

The claim form, or a copy of same, can also be stored by and/or in the respective provider communication device 20, the respective user communication device 40, or the respective intermediary communication device 50. The claim form, or a copy of same, can also be transmitted to and/or stored by and/or in the healthcare records computer 60, in the database 70H of the electronic interactive content/media computer 70, or in the database 80H of the insurance exchange computer 80.

At step 1210, the claim form, or a copy of same, and/or any and/or all pertinent information regarding the claim form, the claim submission, the date and/or the time of same, the individual, the patient, the provider visit, and/or any diagnoses and/or treatments considered, the final diagnosis and/or the prescribed treatment, any diagnostic report(s), treatment report(s), and/or treatment plan(s), can also be stored and the individual's or the patient's electronic healthcare record in the database 10H of the central processing computer 10, and/or can be transmitted to and/or stored in the user communication device 40 where it can be stored in the individual's or the patient's electronic healthcare record in the database 40H of same, and/or can be transmitted to and/or stored in healthcare records computer 60 where it can be stored in the individual's or the patient's electronic healthcare record in the database 60H of same, and/or can be transmitted to and/or stored in electronic interactive content/media computer 70 where it can be stored in the individual's or the patient's electronic healthcare record in the database 70H of same, and/or can be transmitted to and/or stored in database 80H of the insurance exchange computer 80. Any data and/or information contained in the individual's or the patient's electronic healthcare record in the database 10H of the central processing computer 10, in the database 40H of the user or patient communication device 40, and/or in the database 60H of the healthcare records computer 60, can then be automatically updated and can be available for the individual's or the patient's, next office visit, treatment and/or diagnosis.

In another preferred embodiment, the apparatus 100 or the central processing computer 10 can also be programmed to identify the individual-specific or patient-specific educational information or individual-specific or patient-specific instructional information regarding the diagnosis, the treatment, the treatment plan, a prescribed electronic interactive activity or prescribed electronic interactive activities, and/or any information, link(s), or hyperlink(s) relating thereto or pertaining thereto.

In another preferred embodiment, the claim form or the claim submission can be date-stamped and/or time-stamped so that the claim processing corresponding to same can be tracked or monitored so as to facilitate audits of the insurer or payer or insurance company in order to ascertain if the insurer or payer or insurance company is properly and/or efficiently handling a claim or claims for the individual or patient and/or if the insurer or payer or insurance company is in compliance with any laws, rules, or regulations, governing claims processing and/or handling. Information regarding the date stamped and/or time stamped claim forms or claims, including the insurer's or the payer's, or the insurance company's, processing or handling of same, and the response or reply to same, can also be stored in the individual's or the patient's electronic healthcare record in the database 10H of the central processing computer 10, and/or in the database 10H of the central processing computer 10, and can be accessed and/or obtained by any authorized user or entity. In this manner, the apparatus 100 of the present invention can also be used in order to ascertain information regarding the processing and/or handling of claims or claim forms by a respective insurer or payer or insurance company for any individual or patient, or for any group or groups of individuals or patients. The operation of the apparatus 100 will then cease at step 1211.

In another preferred embodiment, the claim form transmitted to the insurer or payer computer 30 can have attached thereto, or can include, a link(s) or a hyperlink(s) to the individual's or the patient's electronic healthcare record, and/or any notes, comments, or messages, entered by an the individual or the patient, a caregiver, or the provider, into the electronic healthcare record. In another preferred embodiment, the claim form transmitted to the insurer or payer computer 30 can also have attached thereto, or can include, or can include a link(s) or a hyperlink(s) to, any x-ray, x-rays, lab result or lab results, MRI image, MRI images, CAT scan image, PET scan image, provider notes, individual, patient, or caregiver, records or information, individual, patient, or caregiver, provided information, and/or any other information which can be obtained by or with any healthcare, medical, or dental, equipment or device, or any other information which may be requested of, or obtained from, a provider or which may be used by an insurer or payer or insurance company in processing, investigating, or evaluating, a claim submission.

In this regard, the claim form can also, for example, contain or include, or can have attached thereto, or can contain a link or links to or a hyperlink or hyperlinks to, a digital or electronic copy of an x-ray or copies of x-rays, an MRI image or MRI images, a CAT scan image or CAT scan images, a PET scan image or images, a copy of any other type of image obtained from any type of healthcare equipment or device, a test result or test results, a blood pressure reading or blood pressure readings, a heart pulse rate or reading, a note or notes regarding the individual, the patient, or the caregiver, a photograph or photographs of the individual, the patient, or the caregiver, a photograph or photographs of an accident scene, information regarding a vital sign or vital signs, or any other information which may be requested of, or obtained from, a provider, or information which may be requested of, or obtained from, the individual, the patient, or the caregiver, or which may be used by a insurer or payer or insurance company in processing, investigating, or evaluating, a claim form or a claim submission or a request for payment.

In another preferred embodiment of the embodiment of FIGS. 12A and 12B, or any and/or all the herein-described embodiments, the apparatus 100 or the central processing computer 10 can provide information regarding ICD, ICD-10, ICD-9, or other, codes, diagnostic codes, symptom codes, treatment codes, billings codes, or any other codes or information useful in entering or recording individual or patient information into a electronic healthcare record or an electronic healthcare file of the individual or the patient. In this manner, the apparatus 100 of the present invention can provide any user, individual, patient, provider, insurer or payer, or intermediary, or any other individual or person, using the apparatus 100 of the present invention, with accurate and current data or information so as to ensure that the information described herein as being stored in the electronic healthcare records or the electronic healthcare files of individuals or the patients, records or files of providers, insurer or payers, third parties, or intermediaries, or any claim forms, request for payments, insurance claims, prescriptions, referrals, or any other information, described herein as being processed, generated, or stored, by the apparatus 100 of the present invention, is accurate, correct, and up-to-date with current standards in the healthcare field or any related fields.

In another preferred embodiment, as well as in any and/or all of the embodiments described herein, the central processing computer 10, or the CPU 10A, in addition to generating any of the herein-described diagnostic reports, treatment reports, or treatment plans, can also generate a wellness report, an exercise report, a fitness report, a nutritional report, a diet report, a rehabilitation report, a therapy report, and/or any other report containing wellness information, exercise information, fitness information, suggested electronic interactive activities, suggested exercises and/or suggested fitness routines, nutritional information, diets, rehabilitation exercises or activities, and/or therapy information. The respective wellness report, exercise report, fitness report, nutritional report, diet report, rehabilitation report, therapy report, and/or any other report, can be generated using data and/or information contained in the individual's or the patient's electronic healthcare record or electronic healthcare file, and/or by using any information regarding any examination findings, procedure findings, symptoms, diagnoses, possible diagnoses, treatments, treatment plans, or any other information used in generating any of the herein-described diagnostic reports, treatment reports, or treatment plans. The respective wellness report, exercise report, fitness report, nutritional report, diet report, rehabilitation report, therapy report, and/or any other report, can also be generated using data and/or information provided by any individual, the patient, or a caregiver, by any provider, by any insurer or payer, or by any intermediary or third party.

The respective wellness report, exercise report, fitness report, nutritional report, diet report, rehabilitation report, therapy report, and/or any other report, can also be generated in connection with the generation of any diagnostic report, treatment report, or treatment plan. In a preferred embodiment, any herein-described final diagnosis and/or prescribed treatment plan, or any diagnostic reports, treatment reports, or treatment plans, can include or contain the respective wellness report, exercise report, fitness report, nutritional report, diet report, rehabilitation report, therapy report, and/or any other report, or can include or contain a link(s) or a hyperlink(s) to the respective wellness report, exercise report, fitness report, nutritional report, diet report, rehabilitation report, therapy report, and/or any other report. Any and/or all of the respective wellness reports, exercise reports, fitness reports, nutritional reports, diet reports, rehabilitation reports, therapy reports, and/or any other reports, can be stored in the individual's or the patient's electronic healthcare record of electronic healthcare file. Any herein-described final diagnosis and/or prescribed treatment plan or any of the herein-described diagnostic reports, treatment reports, or treatment plans, wellness reports, exercise reports, fitness reports, nutritional reports, diet reports, rehabilitation reports, therapy reports, and/or any other reports, can also contain or include a link(s) or hyperlink(s) to a respective individual's or patient's electronic healthcare record or electronic healthcare file, and any of the herein-described electronic healthcare records or electronic healthcare files of a respective individual or patient can contain or include a link(s) or hyperlink(s) to a respective final diagnosis or prescribed treatment plan, diagnostic report, treatment report, or treatment plan, wellness report, exercise report, fitness report, nutritional report, diet report, rehabilitation report, therapy report, and/or any other report.

In another preferred embodiment, any individual, patient, or caregiver, or a provider, insurer or payer, or any intermediary or third party, can also access the central processing computer 10 with a respective communication device 40, 20, 30, or 50 and can request that a respective wellness report, exercise report, fitness report, nutritional report, diet report, rehabilitation report, therapy report, and/or any other report, be generated for the individual or the patient. Thereafter, the central processing computer 10, or the CPU 10A, can generate the respective wellness report, exercise report, fitness report, nutritional report, diet report, rehabilitation report, therapy report, and/or any other report, transmit same to the respective communication device 40, 20, 30, or 50 of the requesting individual, patient, or caregiver, or a provider, insurer or payer, or any intermediary or third party, and/or store same in the electronic healthcare record or the electronic healthcare file of the individual or the patient.

In another preferred embodiment, any of the herein-described diagnostic reports, treatment reports, treatment plans, wellness reports, exercise reports, fitness reports, nutritional reports, diet reports, rehabilitation reports, therapy reports, and/or any other reports, can also include or can contain data and/or information regarding suggested or recommended providers, healthcare facilities, treatment facilities or centers, suggested appointments with providers, lifestyle suggestions, wellness or fitness suggestions, exercise routines or suggestions, diet suggestions, and/or a link(s) or hyperlink(s) to any of the above data and/or information.

In any and/or all of the embodiments described herein, the central processing computer 10, or the CPU 10A, in performing any processing of individual or patient information, diagnosis information, and/or treatment information, described herein, can perform such processing in conjunction with drug and/or other treatment interaction information and/or individual or patient allergy information so as to provide an added safeguard in the diagnosis and treatment planning processes. Any and/or all processing described herein is also performed in conjunction with each individual's or patient's electronic healthcare record and any and/or all data and/or information contained or stored therein, healthcare or medical history,

The apparatus and method of the present invention can also be utilized to provide information regarding any electronic interactive activity or electronic interactive activities with or via an electronic healthcare record, provide access to an electronic interactive activity, provide or administer an electronic interactive activity or electronic interactive activities from or via an electronic healthcare record, provide for the monitoring and/or the recording of data and/or information, as well as any results obtained from and/or recorded, from an administration of an electronic interactive activity or electronic interactive activities, with, by, or using, an electronic healthcare record, or to utilize data and/or information obtained from an electronic interactive activity or electronic interactive activities, in performing follow-up diagnostic processing, treatment planning and/or treatment plan monitoring, and/or any other healthcare information processing and/or healthcare-related processing for or regarding an individual or a patient.

The apparatus and method of the present invention can also be utilized to process data and/or information obtained with or from an electronic interactive activity or electronic interactive activities, provided from or via an electronic healthcare record in performing a diagnosis, in prescribing a treatment plan, in monitoring a treatment plan, in processing information for evaluating a treatment plan, in processing information for or regarding evaluating an individual's or a patient's progress or response to a treatment or a treatment plan, the effectiveness of the treatment or the treatment plan, or the progress or effectiveness of the treatment or treatment plan in meeting or achieving goals related to curing, healing, rehabilitating, training, or re-training, an individual or patient.

The apparatus and method of the present invention can also be utilized to provide and/or administer an electronic interactive activity, record and process data and/or information regarding an electronic interactive activity performed by an individual or a patient, allow for a healthcare provider or any other provider to monitor and/or observe the individual's or the patient's activities in performing an electronic interactive activity, allow for a healthcare provider or any other provider to monitor and/or observe the individual's or the patient's activities in performing an electronic interactive activity in real-time and/or otherwise, provide a recording of the individual's or the patient's activities in performing an electronic interactive activity to a healthcare provider or any other provider, and/or allow for a plurality or individual or patient's to engage in groups activities with or using an electronic interactive activity or electronic interactive activities.

In another preferred embodiment, the apparatus 100 and method of the present invention can be utilized in order to allow an individual or a patient to access any electronic interactive activity prescribed for him or her and to engage in same, use same, perform same, or participate in same, via his or her electronic healthcare record. Any data and/or information regarding the individual's or the patient's accessing of the electronic interactive activity prescribed for him or her, including but not limited to, the date and time of same, and any data and/or information regarding the individual's or the patient's engagement in or with same, use of same, performance of same, or participation in same, including but not limited to, any information provided by the individual or the patient, any information obtained by the apparatus 100 or the central processing computer 10, any information obtained by the apparatus 100 or the central processing computer 10 in monitoring and/or observing the individual's or the patient's engagement with, use of, performance of, or participation in, the electronic interactive activity, and/or any performance results, measurements, or scores, the individual to the patient has achieved in the engagement with, use of, performance of, or participation in, the electronic interactive activity, can be stored by the apparatus 100, the central processing computer 10, and/or the user or patient communication device 40 used by the individual or the patient.

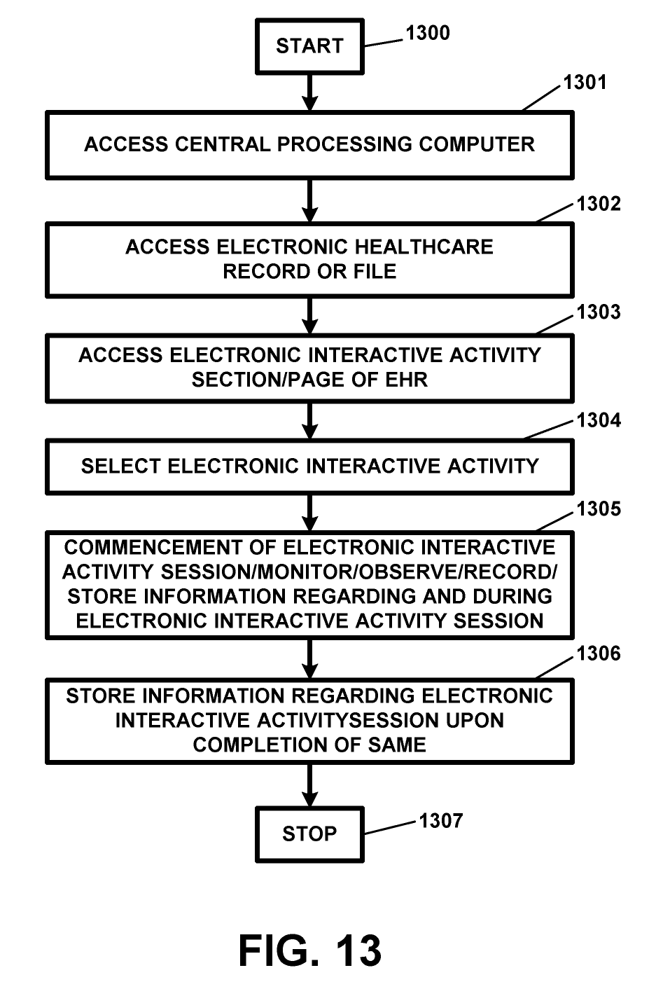

FIG. 13 illustrates another preferred embodiment method of utilizing the apparatus 100 of the present invention, in flow diagram form. With reference to FIG. 13, the operation of the apparatus 100 commences at step 1300. At step 1301, an individual or a patient, for whom an electronic interactive activity has been prescribed, can access the central processing computer 10 using his or her user or patient communication device 40. At step 1302, the individual or the patient can then access his or her electronic healthcare record or electronic healthcare file.

At step 1303, the individual or the patient can then access an electronic interactive activity section or page of his or her electronic healthcare record or electronic healthcare file which, in a preferred embodiment, contains information regarding, as well any link(s) or hyperlink(s) to, any electronic interactive activities which are or can be available to the individual or the patient via the electronic healthcare record or electronic healthcare file. In a preferred embodiment, the electronic interactive activity section or page can contain or include information regarding, or a link, or a hyperlink, to, any electronic interactive activity or any electronic interactive activities which have been prescribed for the individual or the patient, or any electronic interactive activity or any electronic interactive activities which have been suggested for the individual or the patient, or which have otherwise been identified as being beneficial for the individual or the patient.

At step 1304, the individual or the patient can select the electronic interactive activity with, or in, which he or she desires to access and engage, use, perform, or participate. In a preferred embodiment, the electronic interactive activity can be a video game, an audio game, a video and audio game, an electronic or digital exercise, an electronic or digital puzzle or activity, an electronic or digital diagnostic activity or tool, an electronic or digital treatment, treating planning, or therapy, activity or tool, an electronic or digital mental health or behavioral health activity or tool, an electronic or digital physical or physical therapy activity or tool, an electronic or digital training activity or tool, an electronic or digital rehabilitation activity or tool, an electronic or digital mental health or behavioral health training or re-training activity or tool, an electronic or digital physical training or re-training activity or tool, a survey, a questionnaire, or any other game, exercise, activity, information gathering event or occurrence.

In a preferred embodiment, as and for an example, the electronic interactive activity selected by the individual or the patient can be a video game. Although a video game is described as being selected in the preferred embodiment, it is important to note that the selected electronic interactive activity could also be an audio game, a video and audio game, an electronic or digital exercise, an electronic or digital puzzle or activity, an electronic or digital diagnostic activity or tool, an electronic or digital treatment, treating planning, or therapy, activity or tool, an electronic or digital mental health or behavioral health activity or tool, an electronic or digital physical or physical therapy activity or tool, an electronic or digital training activity or tool, an electronic or digital rehabilitation activity or tool, an electronic or digital mental health or behavioral health training or re-training activity or tool, an electronic or digital physical training or re-training activity or tool, a survey, a questionnaire, or any other game, exercise, activity, information gathering event or occurrence. Thereafter, the operation of the apparatus 100 will proceed to step 1305.

At step 1305, the electronic interactive activity session will begin and the individual or the patient can thereafter engage in, use, perform, or participate in, the selected electronic interactive activity. In a preferred embodiment, in which a video game has been selected as the electronic interactive activity, the individual or the patient can play the video game. At step 1305, the central processing computer 10 can monitor and/or observe and record and store any data and/or information regarding the electronic interactive activity session, including the time and date of same, the duration of same, the identity of the individual or the patient, and/or any performance results, measurements, or scores, the individual to the patient has achieved and/or any other data and/or information regarding the individual's or the patient's activities, actions, reactions, during, pertaining to, or otherwise related to, the individual's or the patient's activities and/or performance during the electronic interactive activity session.

At step 1305, the individual or the patient will continue to engage in the electronic interactive activity session until the session is ended or completed by the central processing computer 10, such as when electronic interactive activity session ends in or as a normal electronic interactivity completion, or such as when the individual or the patient decides to terminate the electronic interactive activity session prematurely or otherwise. At step 1305, the central processing computer 10 will record and store data and/or information regarding whether or not the electronic interactive activity session ended normally or whether or not the individual or the patient terminated the electronic interactive activity session prematurely or otherwise.

Once the electronic interactivity session has ended or has been completed or terminated, the operation of the apparatus 100 will proceed to step 1306 and the central processing computer 10 will store all data and/or information regarding the electronic interactive activity session described herein as being recorded and stored by the central processing computer 10 at step 1305. At step 1306, the central processing computer will also store any and/or all data and/or information described as being recorded and stored by the central processing computer 10 at step 1305, in the individual's or the patient's electronic healthcare record or electronic healthcare file. In a preferred embodiment, at step 1306, the central processing computer 10 can also generate an electronic interactive activity session message containing information regarding the individual's or the patient's having engaged in the electronic interactive activity session which can contain or include the name of the individual or the patient, the date and time of the electronic interactive activity session, the electronic interactive activity accessed, engaged in, used, performed, or participated in, during the electronic interactive activity session, and/or any of the data and/or information recorded and stored by the central processing computer during and/or related to the electronic interactive activity session.

At step 1306, the central processing computer 10 can also transmit the electronic interactive activity session message to the provider communication device 20 of the healthcare provider of the individual or the patient, to any number of provider communication devices 20 of any number of the healthcare providers of the individual or the patient. At step 1306, the central processing computer 10 can also transmit the electronic interactive activity session message to the payer communication device 30 of the individual's or the patient's insurer or payer. At step 1306, the central processing computer 10 can also transmit the electronic interactive activity session message to the user or patient computer 40 of or associated with the individual or the patient or a caregiver of the individual or the patient. In a preferred embodiment, the electronic interactive activity session message can be transmitted as, in, or attached to, an electronic mail message, a text message, an SMS message, or any other electronic transmission or electronic data transmission. Thereafter, the operation of the apparatus 100 will cease at step 1307.

In another preferred embodiment, the embodiment of FIG. 13 can also be utilized in order to allow for group participation by a plurality of individuals or patients in an electronic interactive activity or to allow a group of individuals or patients to access and engaged in, use, perform, or participate in, a same electronic interactive activity with each other and/or at the same time. In a preferred embodiment, for example, any group participants can be identified from any social network(s) or social networking groups of individuals or patients who may either have, be suspected of having, or be under the observation or care for or regarding, a same illness, sickness, injury, condition, syndrome, state of dysfunction, and/or any healthcare or healthcare-related problem or impairment.

In a preferred embodiment, each of the individuals or patients can gain access to the electronic interactive activity section via his or her electronic healthcare record or electronic healthcare file using his or her user or patient communication device 40 and can engaged in, use, perform, or participate in, same. In such a preferred embodiment, the central processing computer 10 can record and store, for each individual or patient, any and/or all of the data and/or information, described herein as being recorded and stored by the central processing computer 10 in the embodiment of FIG. 13, for each individual or patient who participates in the electronic interactive activity session.

In another preferred embodiment, any healthcare provider of the individual or patient, or of any other individual or patient who engages in, uses, performs, or participates in, the electronic interactive activity section, can access the electronic interactive activity section via his or her provider communication device 20 and can monitor and/or observe the activity of the individual or the patient. The healthcare provider can also make and/or enter, into the individual's or the patient's electronic healthcare record or electronic healthcare file, any notes, comments, or observations, regarding the individual or the patient, or any notes, comments, or observations, about or regarding the individual's or the patient's actions, engagement, use, performance, or participation, in or during the electronic interactive activity section at any time before, during, or after, the electronic interactive activity section.

In the case where a plurality of individuals or patients engage in, use, perform, or participate in, the electronic interactive activity section, a healthcare provider of or for each individual or patient can access the electronic interactive activity section and monitor and/or observe the activity of the individual or patient he or she seeks to monitor and/or observe and/or to monitor and/or observe the activity of the individual or the patient under his or her care. In such an embodiment, any healthcare provider can also make and/or enter, into any individual's or patient's electronic healthcare record or electronic healthcare file, any notes, comments, or observations, regarding that individual or patient, or any notes, comments, or observations, about or regarding that individual's or patient's actions, engagement, use, performance, or participation, in or during the electronic interactive activity section at any time before, during, or after, the electronic interactive activity section. In this manner, any notes, comments, or observations, made by any other healthcare provider, other than the healthcare provider of the individual or the patient, can be considered and/or utilized in diagnosing or treating the individual or the patient.

In a preferred embodiment, if the individual or the patient, or if any individual or patient of a group electronic interactive activity session, desires to have his or her healthcare provider be notified regarding his or her involvement in any electronic interactive activity session in which he or she is participating or requests that his or her healthcare provider monitor and/or observe any electronic interactive activity session in which he or she is participating, the respective individual or patient can post a respective instruction or request for same with the central processing computer 10 and/or with or in his or her electronic healthcare record or electronic healthcare file and the central processing computer 10 can automatically generate a respective provider notification message or a provider monitoring request message which can then be automatically transmitted to the provider communication device 20 of the provider of the respective individual or patient. Thereafter, the provider can access the electronic interactive activity session and can monitor and/or observe the activity or the respective individual or patient.

In another preferred embodiment, the apparatus 100 of FIG. 13 can, at step 1306, automatically utilize any data and/or information recorded and stored for the individual or the patient at step 1305 and at step 1306 in order to automatically perform a diagnostic evaluation or analysis of the individual or the patient using data and/or information stored in the individual's or the patient's electronic healthcare record or electronic healthcare file and the data and/or information recorded and stored from the electronic interactive activity session at steps 1305 and 1306. In a preferred embodiment, the apparatus 100 can also, at step 1306, perform a diagnostic evaluation or analysis of the individual or the patient and generate a diagnostic report as described herein at step 1204 of the embodiment of FIGS. 12A and 12B.

In another preferred embodiment, the apparatus 100 of FIG. 13 can also, at step 1306, automatically utilize any data and/or information recorded and stored for the individual or the patient at step 1305 and at step 1306, along with any data and/or information contained in any diagnostic report generated at step 1306 in order to automatically generate a treatment report and a treatment plan for any diagnosis or list of possible diagnoses identified in the diagnostic report which is generated at step 1306. In a preferred embodiment, the apparatus 100 can also, at step 1306, generate a treatment report and/or a treatment plan for the individual or the patient as described herein at step 1205 of the embodiment of FIGS. 12A and 12B.

In another preferred embodiment, the apparatus 100 and method of the present invention can be utilized by a healthcare provider of the individual or the patient to access and/or to review, via the individual's or the patient's electronic healthcare record or electronic healthcare file, any data and/or information regarding or obtained from or during any electronic interactive activity session or any number of electronic activity sessions in which the individual or the patient was involved and/or any performance results, measurements, or scores, of or achieved by the individual or the patient regarding or relating to same, to make or enter any notes, comments, or observations, regarding same, to perform an updated diagnostic evaluation of the individual or the patient, and/or to generate an updated diagnostic report, treatment report, or treatment plan(s), for the individual or the patient, based on or using any of the data and/or information regarding or obtained from or during any electronic interactive activity session in which the individual or the patient was involved or any notes, comments, or observations, made or provided by the healthcare provider.

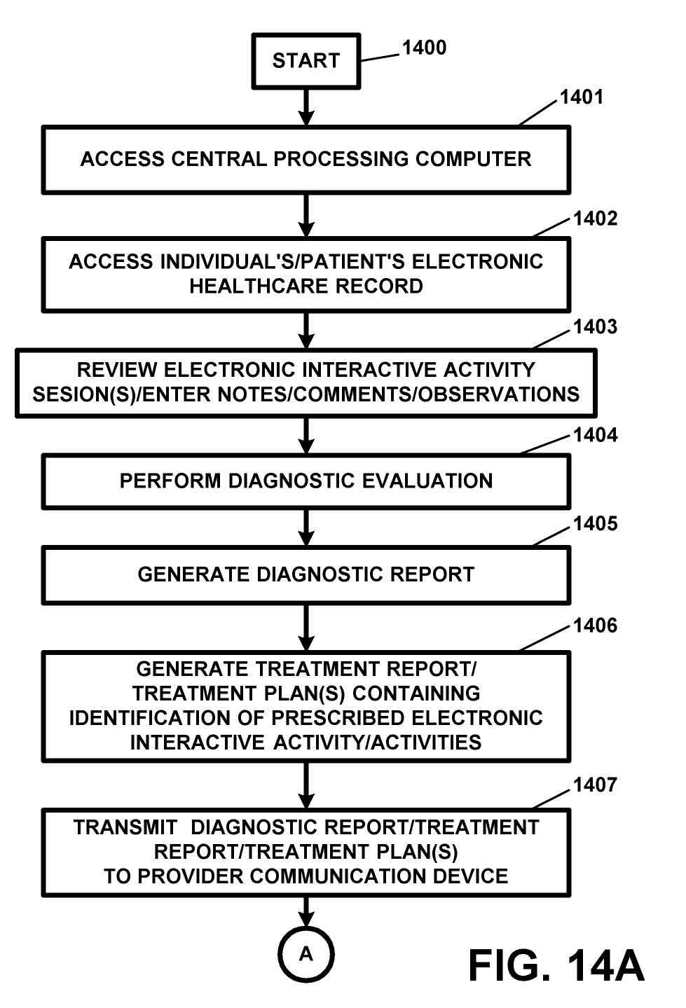

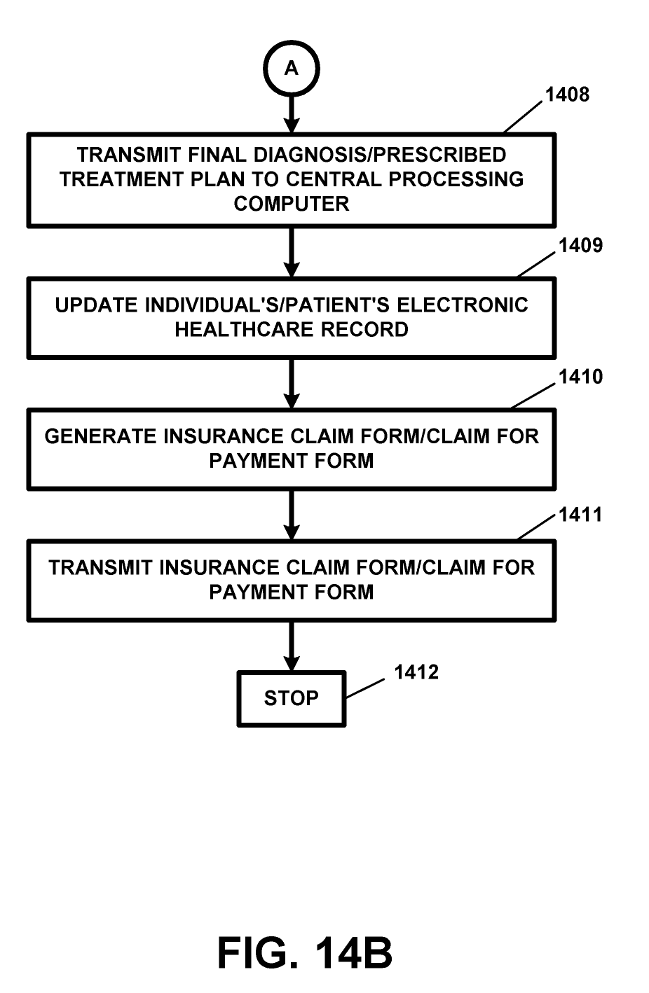

FIGS. 14A and 14B illustrate another preferred embodiment method of utilizing the apparatus 100 of the present invention, in flow diagram form. With reference to FIGS. 14A and 14B, the operation of the apparatus 100 commences at step 1400. At step 1401, the healthcare provider of an individual or patient can access the central processing computer 10 via or using his or her provider communication device 20. At step 1402, the healthcare provider can access the electronic healthcare record of the individual or the patient. At step 1403, the healthcare provider can access and/or review any data and/or information regarding or obtained from or during any electronic interactive activity session or any number of electronic interactive activity sessions in which the individual or the patient was involved and/or any performance results, measurements, or scores, of or achieved by the individual or the patient regarding or relating to same.

At step 1403, the healthcare provider can also make or enter, into his or her provider communication device 20, any notes, comments, or observations, regarding any data and/or information regarding or obtained from or during any electronic interactive activity session or from or during any number of electronic interactive activity sessions in which the individual or the patient was involved and/or any performance results, measurements, or scores, of or achieved by the individual or the patient regarding or relating to same. At step 1403, the healthcare provider can transmit his or her notes, comments, or observations, from his or her provider communication device 20 to the central processing computer 10 which will receive and process same and store same in the individual's or the patient's electronic healthcare record or electronic healthcare file. The operation of the apparatus 100 will then proceed to step 1404.

At step 1404, the central processing computer 10, or the CPU 10A can perform a diagnostic evaluation of the individual or the patient, in a same, a similar, or an analogous, manner as described in step 1203 of the embodiment of FIGS. 12A and 12B, utilizing any data and/or information regarding or obtained from or during any electronic interactive activity session or from or during any number of electronic interactive activity sessions and/or any notes, comments, or observations, made and/or entered by the healthcare provider relating to or regarding same.

At step 1405, the central processing computer 10, or the CPU 10A, can generate a diagnostic report, which can include a diagnosis of the individual's or the patient's condition, in a same, a similar, or an analogous, manner as described in step 1204 of the embodiment of FIGS. 12A and 12B. The diagnostic report, which is generated at step 1405, can include a single diagnosis and/or a list of possible diagnoses, along with their respective probabilities of being accurate, which may pertain to the individual's or the patient's condition. In a preferred embodiment, the diagnostic report can also contain information identifying an electronic interactive activity or any number of electronic interactive activities which might identified by the central processing computer 10 or the CPU 10A as being deemed to be useful, desired, or required, in conducting further a diagnostic evaluation or analysis of the individual or the patient. In a preferred embodiment, at step 1405, the central processing computer 10, or the CPU 10A, can utilize data and/or information contained in the database 10H in order to identify any other or additional electronic interactive activity or any electronic interactive activities which can be utilized in performing a diagnostic evaluation of the individual or the patient.

In a preferred embodiment, for example, the central processing computer 10, or the CPU 10A, can process data and/or information regarding or obtained from or during any electronic interactive activity session or from or during any number of electronic interactive activity sessions and/or any notes, comments, or observations, made and/or entered by the healthcare provider in conjunction with any data and/or information regarding any symptoms, examination findings, or observations, whether previously known or recently learned and/or any other data and/or information stored in the database 10H which can or may relate or correlate any electronic interactive activity or any electronic interactive activities to any diagnosis or diagnostic evaluation for or regarding any symptoms, diseases, conditions, illnesses, syndromes, sicknesses, and/or states of dysfunction, relating to the symptoms, if any, and/or the examination findings or observations in order to identify any, any other, or any additional, electronic interactive activity or any electronic interactive activities which can utilized in performing a further diagnostic evaluation of the individual or the patient.

At step 1406, the central processing computer 10 can then generate a treatment report, in a same, a similar, or an analogous, manner as described in step 1205 of the embodiment of FIGS. 12A and 12B, which can or which will outline and/or prescribe a treatment(s) for the single diagnosis and/or a treatment(s) for each of the diagnoses in list of possible diagnoses. In a preferred embodiment, the central processing computer 10, at step 1406, in generating the treatment report, can also identify any, any other, or any additional, electronic interactive activity or can identify any number of electronic interactive activities which can or which may be appropriate or suitable in treating the individual or the patient, in performing further diagnostic activities, or in providing therapy, rehabilitation, training, or re-training, for the individual's of the patient's diagnosis and/or treatment regimen.

In a preferred embodiment, the treatment report can also contain information identifying any, any other, or any additional, electronic interactive activity or any number of electronic interactive activities which might identified by the central processing computer 10 or the CPU 10A as being deemed to be useful, desired, or required, in treating the diagnosis or each of the possible diagnoses identified in the diagnostic report. In a preferred embodiment, at step 1406, the central processing computer 10, or the CPU 10A, can also utilize data and/or information contained in the database 10H in order to identify any, any other, or any additional, electronic interactive activity or any electronic interactive activities which can be utilized in treating the diagnosis or each of the possible diagnoses identified in the diagnostic report. In a preferred embodiment, for example, the central processing computer 10, or the CPU 10A, can process data and/or information regarding any identified diagnosis in conjunction with any data and/or information stored in the database 10H which relates or correlates any electronic interactive activity or any electronic interactive activities to the identified diagnosis in order to identify any electronic interactive activity or electronic interactive activities which can be utilized in treating the identified diagnosis or in treating each identified diagnosis in the list of possible diagnoses.

At step 1406, the central processing computer 10 can also generate a treatment plan for the diagnosis or for each diagnosis in the list of diagnoses in a same, a similar, or an analogous, manner as described in step 1205 of the embodiment of FIGS. 12A and 12B. In a preferred embodiment, the central processing computer 10, at step 1406, in generating the treatment plan, can identify any, any other, or any additional, electronic interactive activity or can identify any number of electronic interactive activities which can or which may be appropriate or suitable in treating the individual or the patient, in performing further diagnostic activities, or in providing therapy, rehabilitation, training, or re-training, for the individual's of the patient's diagnosis and/or treatment regimen. In a preferred embodiment, the central processing computer 10, or the CPU 10A, when generating the treatment report and/or the treatment plan, or each treatment report or each treatment plan, can also process any information regarding or contained in same in conjunction with any information regarding any physical limitations, mental health limitations or behavioral limitations, emotional limitations, or any other limitations, known to be associated with or regarding the individual or the patient, and/or the central processing computer 10 can also process any information regarding or contained in same in conjunction with any information regarding any possible drug interactions and/or treatment interactions, and/or individual or patient allergies, as well as any treatments, medications, therapies, or tests, or any other activities, which might prove to be harmful to the individual or the patient.

In a preferred embodiment, the treatment plan, generated at step 1406, can also contain information identifying an electronic interactive activity or any number of electronic interactive activities which might identified by the central processing computer 10 or the CPU 10A as being deemed to be useful, desired, or required, in treating the diagnosis or each of the possible diagnoses identified in the diagnostic report. In a preferred embodiment, at step 1406, the central processing computer 10, or the CPU 10A, can utilize data and/or information contained in the database 10H in order to identify any electronic interactive activity or any electronic interactive activities which can be utilized in treating the diagnosis or each of the possible diagnoses identified in the diagnostic report. In a preferred embodiment, for example, the central processing computer 10, or the CPU 10A, can process data and/or information regarding any identified diagnosis in conjunction with any data and/or information stored in the database 10H which relates or correlates any electronic interactive activity or any electronic interactive activities to the identified diagnosis in order to identify any electronic interactive activity or electronic interactive activities which can be utilized in treating the identified diagnosis or in treating each identified diagnosis in the list of possible diagnoses.

In a preferred embodiment, the treatment report and/or the treatment plan can include information regarding a plan of treatment, including, but not limited to, any and/or all electronic interactive activities in which the individual or the patient should engage in, perform, or participate in, as well as any necessary, recommended, or suggested, doctor visits, office visits, provider visits, hospital stays or stays in healthcare facilities or rehabilitative facilities, tests, testing, procedures, treatments, surgeries, consultations, therapy sessions, and/or rehabilitation sessions, and/or or any medications, drugs, alternative medicine treatments or products, goods, or services, and/or any other goods, products, or services, and/or any other healthcare or other services or goods, which may be prescribed, needed, or deemed desirable, for that treatment plan, which are or which might be called for by a respective treatment report or treatment plan.

In a preferred embodiment, as well as in any and/or all of the embodiments described herein, any of the herein-described providers, suppliers, or vendors, of any of the herein-described electronic interactive activities, can access the central processing computer 10 at any time using his, her, or its, respective provider communication device 20 or intermediary communication device 50, and can transmit or upload, to the central processing computer 10 and/or to the electronic interactive content/media computer 70, his, her, or its, respective electronic interactive activity or electronic interactive activities.

In another preferred embodiment, the treatment report and/or the treatment plan, or each treatment report or treatment plan corresponding to each possible diagnosis, can also contain or include information regarding, and/or a link(s) or a hyperlink(s) to, any of the electronic interactive activities identified in the respective treatment report or treatment plan, along with any information, or any link or hyperlink to any information for or regarding each electronic interactive activity including, but not limited to, instructions for using same, purpose of same in the course of treatment, and/or any other pertinent information regarding same. In another preferred embodiment, the treatment report and/or the treatment plan, or each treatment report or treatment plan corresponding to each possible diagnosis, can also contain or include information regarding the prescribed used of any electronic interactive activity or electronic interactive activities, prescribed number of times for using same, prescribed usage frequency, and/or any other pertinent information regarding how a respective electronic interactive activity is to be utilized or employed in the treatment plan or the treatment process. In another preferred embodiment, the treatment report and/or the treatment plan, or each treatment report or treatment plan corresponding to each possible diagnosis, can also contain or include information regarding, and/or a link(s) or a hyperlink(s) to, any of the providers, suppliers, or vendors, of any of the prescribed electronic interactive activities or to any computers, communication devices, or websites, of or associated with same.

In another preferred embodiment, the treatment report and/or the treatment plan, or each treatment report or treatment plan corresponding to each possible diagnosis, can also contain or include information regarding, and/or a link(s) or a hyperlink(s) to, a provider communication device 20 of each of the individual's or the patient's healthcare providers or doctors and/or to healthcare providers or doctors identified as being able to treat the individual or the patient, so that the individual or the patient, or a caregiver, can contact or communicate with a respective healthcare provider or doctor, or so that the individual or the patient, or a caregiver, can schedule an appointment with the healthcare provider or doctor via the central processing computer 10 or otherwise, or so that the individual or the patient can utilize the information or the link(s) or hyperlink(s) for any other appropriate reason.

In another preferred embodiment, the treatment report and/or the treatment plan, or each treatment report or treatment plan corresponding to each possible diagnosis, can also contain or include information regarding, and/or a link(s) or a hyperlink(s), to an insurer communication device 30 of each insurer or payer of the individual or patient so that the individual or the patient, or a caregiver, can contact or communicate with a respective healthcare insurer or payer, so that the individual or the patient can submit an insurance claim to the insurer or payer via the central processing computer 10 or otherwise, so that the individual or the patient can submit a pre-authorization request to the insurer or payer, or so that the individual or the patient can utilize the information or the link(s) or hyperlink(s) for any other appropriate reason.

In another preferred embodiment, the treatment report and/or the treatment plan, or each treatment report or treatment plan corresponding to each possible diagnosis, can also contain or include information regarding, and/or a link(s) or a hyperlink(s), to the central processing computer 10 and/or to the healthcare records computer 60 so that the individual or the patient can access his or her electronic healthcare record(s) or any portion of same. In another preferred embodiment, the treatment report and/or the treatment plan, or each treatment report or treatment plan corresponding to each possible diagnosis, can also contain or include information regarding, and/or a link(s) or a hyperlink(s), to the central processing computer 10 and/or to the electronic interactive content/media computer 70 of or associated with any provider, supplier, or vendor, of any prescribed electronic interactive activity prescribed therein, so that the individual or the patient. In another preferred embodiment, the treatment report and/or the treatment plan, can also contain or include information regarding, and/or a link(s) or a hyperlink(s), to the insurance exchange computer 80 so that the individual or the patient can access the insurance exchange computer 80 in order file a healthcare insurance claim, check on the status of a healthcare insurance claim, or purchase a healthcare or other insurance policy.

In another preferred embodiment, the treatment report and/or the treatment plan, or each treatment report or treatment plan corresponding to each possible diagnosis, can also contain or include information regarding, and/or a link(s) or a hyperlink(s), to a social networking computer 90 and/or to any social networking web site or web pages, and/or to or for any social networking members, support groups, social networking support group members, on-line seminars, social networking lectures, classes, or seminars, social networking sponsored lectures, classes, or seminars, social networking discussions, question and answer sessions, or informational or other forums, or any other social networking or social networking sponsored activities or events, forums, chat room discussions, or any other information providing sources, with which or with whom the individual or the patient may engage. The providing of the social networking information to the individual or the patient can serve to allow the individual or the patient to learn more about a diagnosis, a treatment, or a treatment plan, or to interact with others who have been diagnosed with the same or a similar ailment, illness, sickness, or condition, or to interact with others who may be undergoing the same or a similar treatment, or who may be following a same or a similar treatment plan.

In another preferred embodiment, the treatment report or the treatment plan can also contain or include information prescribing that the individual or the patient engage in an electronic interactive activity or electronic interactive activities along with other individuals or patients or in a group electronic interactive activity or in group electronic interactive activities.

In another preferred embodiment, the treatment report and/or the treatment plan, or each treatment report or treatment plan corresponding to each possible diagnosis, can also contain or include information regarding, and/or a link(s) or a hyperlink(s), to a media computer 95 so that the individual or the patient can access news articles or other information regarding his or her diagnosis and/or treatment plan and/or regarding any of the herein-described electronic interactive activities which may be provided by the apparatus 100 and method of the present invention.

In a preferred embodiment, any of the herein-described diagnostic reports, treatment reports, or treatment plans, can also include or can contain data and/or information stored in the individual's or the patient's electronic healthcare record, a link(s) or a hyperlink(s) to data and/or information stored in the individual's or the patient's electronic healthcare record, any notes, comments, or messages, entered by the individual or the patient, or by a caregiver, or by a provider, into the individual's or the patient's electronic healthcare record, and/or any link(s) or hyperlink(s) to any notes, comments, or messages, entered by the individual or the patient, or by a caregiver, or by a provider, into the individual's or the patient's electronic healthcare record.

At step 1407, the central processing computer 10, in a same, a similar, or an analogous manner, as in step 1206 of the embodiment of FIGS. 12A and 12B, will transmit the diagnostic report and/or the treatment report and/or the treatment plan(s) for the diagnosis or for each of the possible diagnoses to the provider's provider communication device 20, at which point the provider, or the healthcare provider or medical doctor, can obtain the diagnosis or list of possible diagnoses and the corresponding treatment report(s) or treatment plan(s) for same. The provider, or the healthcare provider or medical doctor, can then review the diagnostic report and/or the treatment report(s) and/or the treatment plan(s), and choose, select, or decide upon, a final diagnosis and/or a prescribed treatment plan to administer to the individual or the patient.

At step 1408, the provider, or the healthcare provider or medical doctor, will transmit the final diagnosis and the prescribed treatment or prescribed treatment plan, including the prescribed treatment and/or the treatment plan corresponding to same, to the central processing computer 10, in a same, a similar, or an analogous manner, as in step 1207 of the embodiment of FIGS. 12A and 12B. In another preferred embodiment, at step 1408, the provider, or the healthcare provider or medical doctor, or any other provider using the apparatus 100, can also generate and/or transmit a prescription or an electronic prescription for an electronic interactive activity or for any number of electronic interactive activities, a medication, a medicine, or a drug, or for a procedure, a test, an analysis, an analysis work-up, blood work, a treatment, or a therapy, or can generate and/or transmit a referral to another provider, for submission or transmission to the respective pharmacy or other provider, and for storage in the individual's, the patient's electronic healthcare record or electronic healthcare file.

In another preferred embodiment, the final diagnosis, along with the prescribed treatment plan, and the diagnostic report, the treatment report, and/or the treatment plan, generated for and corresponding to same can also be transmitted by the central processing computer 10, at step 1408, to the provider communication device 20 associated with or used by the provider for the provider's records, to a provider communication device 20 associated with or used by another provider of the individual or the patient, to a payer or insurer communication device 30 associated with or used by the insurer or payer of the individual or the patient, to the user communication device 40 associated with or used by the individual or the patient, or a caregiver, or to an intermediary communication device 50 associated with or used by a third party or an intermediary.

In a preferred embodiment, the central processing computer 10 can transmit the final diagnosis along with the prescribed treatment plan, and the diagnostic report, the treatment report, and/or the treatment plan, generated for and corresponding to same to a respective provider communication device 20, to a respective insurer or payer communication device 30, to a respective user communication device 40, or to a respective intermediary communication device 50, either directly, such as via any suitable electronic or other transmission, or indirectly, such as via an e-mail server in the case when the final diagnosis and/or the prescribed treatment plan, and the diagnostic report, the treatment report, and/or the treatment plan, generated for and corresponding to same, can be, can be included in, or can be attached to, an e-mail message or a text message, as SMS message, or any other electronic communication or transmission.

In a preferred embodiment, the final diagnosis along with the prescribed treatment plan, and the diagnostic report, the treatment report, and/or the treatment plan, generated for and corresponding to same, or a respective copy of same can be data-stamped and/or time-stamped.

In a preferred embodiment, the final diagnosis along with the prescribed treatment plan, and the diagnostic report, the treatment report, and/or the treatment plan, generated for and corresponding to same, or a respective copy of same, can also be stored by and/or in the database 20H of the respective provider communication device(s) 20, in the database 30H of respective insurer or payer communication device(s) 30, in the database 40H of the respective user communication device(s) 40, in the database 50H of the respective intermediary communication device(s) 50, in the database 60H the healthcare records computer 60, in the database 70H of the electronic interactive content/media computer 70, and/or in the database 80H of the insurance exchange computer 80.

In a preferred embodiment, the final diagnosis along with the prescribed treatment plan, and the diagnostic report, the treatment report, and/or the treatment plan, generated for and corresponding to same or a respective copy of same, can also be transmitted to and/or stored by or in the individual's or the patient's electronic healthcare record in the database 10H of the central processing computer 10, in the database 20H in the provider communication device 20, in the database 30H of the payer communication device 30, in the database 40H of the user communication device 40, or in the individual's, the patient's, or the caregiver's, electronic healthcare record in the database 60H of the healthcare records computer 60, in the database 70H of the electronic interactive content/media computer 70, or in the database 80H of the insurance exchange computer 80.

In another preferred embodiment, the final diagnosis along with the prescribed treatment plan, and the diagnostic report, the treatment report, and/or the treatment plan, generated for and corresponding to same, can also contain individual-specific or patient-specific educational information or individual-specific or patient-specific instructional information, or a link(s) or a hyperlink(s) to same, regarding the diagnosis, the treatment, or the treatment plan, or information relating thereto, so as to provide educational information or instructional information to the individual or the patient. The individual or the patient, or a caregiver, can thereafter access or obtain the educational information or instructional information via the apparatus 100 and method of the apparatus 100 of the present invention. In this manner, the apparatus 100 and method of the present invention can provide individual-specific or patient-specific educational information or individual-specific or patient-specific instructional information regarding the diagnosis, the treatment, or the treatment plan.

In another preferred embodiment, the apparatus 100 or the central processing computer 10 can be programmed to identify the individual-specific or patient-specific educational information or individual-specific or patient-specific instructional information regarding the diagnosis, the treatment, or the treatment plan, and/or any information, link(s), or hyperlink(s) relating thereto or pertaining thereto.

At step 1409, the central processing computer 10, or the CPU 10A, will then update the individual's or the patient's electronic healthcare record in the database 10H so as to include all of the data and information described as being processed and/or generated by the central processing computer 10, or the CPU 10A, including, but not limited to the individual's or the patient's symptoms or examination findings or any observations, the final diagnosis, the prescribed treatment plan, the diagnostic report(s), the treatment report(s), and/or the treatment plan(s) described as being generated herein, and/or any data and/or information contained in or regarding the final diagnosis along with the prescribed treatment plan, and the diagnostic report(s), the treatment report(s), and/or the treatment plan(s), generated for and/or corresponding to same.

Thereafter, at step 1410, the central processing computer 10, or the CPU 10A, will generate an insurance claim form or a request for payment form (hereinafter “claim form”) which can meet the formal claim submission requirements of the individual's or the patient's insurer, insurance company, or payer. In a preferred embodiment, the claim form can include or can contain any data and/or information which is stored in individual's or the patient's electronic healthcare record, a link(s) or a hyperlink(s) to the individual's or the patient's electronic healthcare record, a link(s) or a hyperlink(s) to any data and/or information stored in the individual's or the patient's electronic healthcare record, any data and/or information regarding or obtained from or during any electronic interactive activity session or from or during any number of electronic interactive activity sessions and/or any notes, comments, or observations, made and/or entered by the healthcare provider relating to or regarding same and/or any link(s) or hyperlink(s) to same, or any notes, comments, or messages, entered by the individual, the patient, or a caregiver, or a provider, into the individual's or the patient's electronic healthcare record, and/or any link(s) or hyperlink(s) to any notes, comments, or messages, entered by the individual, the patient, or a caregiver, or a provider, into the individual's or the patient's electronic healthcare record. In this regard, in a preferred embodiment, any data and/or information contained or included in the individual's or the patient's electronic healthcare record, including any data and/or information regarding or obtained from or during any electronic interactive activity session or from or during any number of electronic interactive activity sessions and/or any notes, comments, or observations, made and/or entered by the healthcare provider relating to or regarding same and/or any link(s) or hyperlink(s) to same, can be readily available to and/or can be readily accessible to or by any insurer or payer, or any employee or agent of same, or by any other user, person, or entity, who or which reviews the claim form or is to review the claim form.

In a preferred embodiment, the claim form can also contain or include, or contain a link(s) or a hyperlink(s) to data and/or information regarding or obtained from or during any electronic interactive activity session or from or during any number of electronic interactive activity sessions and/or any notes, comments, or observations, made and/or entered by the healthcare provider relating to or regarding same, the final diagnosis, the prescribed treatment plan, the diagnostic report(s), the treatment report(s), and/or the treatment plan(s) described as being generated herein, and/or any data and/or information contained in or regarding the final diagnosis along with the prescribed treatment plan, and the diagnostic report(s), the treatment report(s), and/or the treatment plan(s), generated for and/or corresponding to same. In another preferred embodiment, the claim form can also contain or include, or contain a link(s) or a hyperlink(s) to any electronic interactive activity or any electronic interactive activities identified or prescribed in any of the herein-described diagnostic report(s), treatment report(s), and/or treatment plan(s). It is important to note that, in a preferred embodiment, if needed, any diagnostic report described herein as being generated by the central processing computer 10 or the CPU 10A, or by the apparatus, can also prescribe use of, and/or identify, any, any other, or any additional, electronic interactive activity or any electronic interactive activities which might be identified as being useful in performing a further diagnostic evaluation or analysis of the individual or the patient.

At step 1411, the claim form can be automatically transmitted or submitted by or from the central processing computer 10 to the respective insurer or payer communication device 30 of the respective insurer or payer of the individual or the patient. In another preferred embodiment, the claim form, or a copy of same, can also be transmitted by the central processing computer 10, at step 1411, to the provider, to the individual or the patient, or to a third party or intermediary, by transmitting the claim form, or a copy of same, and/or any of the herein-described included or attached information, to a respective provider communication device 20 associated with or used by the provider or any other provider of the individual or patient, to a respective user communication device 40 associated with or used by the individual or the patient, or to a caregiver for the individual or the patient, or to a respective intermediary communication device 50 associated with or used by an intermediary or third party, either directly, such as via any suitable electronic or other transmission, or indirectly, such as via an e-mail server in the case when the copy of the claim form is included in, or is attached to, an e-mail message, a text message, an SMS message, or any other electronic transmission.

The claim form, or a copy of same, can also be stored by and/or in the respective provider communication device 20, the respective user communication device 40, or the respective intermediary communication device 50. The claim form, or a copy of same, can also be transmitted to and/or stored by and/or in the healthcare records computer 60, in the database 70H of the electronic interactive content/media computer 70, or in the database 80H of the insurance exchange computer 80.

At step 1411, the claim form, or a copy of same, and/or any and/or all pertinent information regarding the claim form, the claim submission, the date and/or the time of same, the individual, the patient, the provider visit, and/or any diagnoses and/or treatments considered, the final diagnosis and/or the prescribed treatment, any diagnostic report(s), treatment report(s), and/or treatment plan(s), can also be stored and the individual's or the patient's electronic healthcare record in the database 10H of the central processing computer 10, and/or can be transmitted to and/or stored in the user communication device 40 where it can be stored in the individual's or the patient's electronic healthcare record in the database 40H of same, and/or can be transmitted to and/or stored in healthcare records computer 60 where it can be stored in the individual's or the patient's electronic healthcare record in the database 60H of same, and/or can be transmitted to and/or stored in electronic interactive content/media computer 70 where it can be stored in the individual's or the patient's electronic healthcare record in the database 70H of same, and/or can be transmitted to and/or stored in database 80H of the insurance exchange computer 80. Any data and/or information contained in the individual's or the patient's electronic healthcare record in the database 10H of the central processing computer 10, in the database 40H of the user or patient communication device 40, and/or in the database 60H of the healthcare records computer 60, can then be automatically updated and can be available for the individual's or the patient's, next office visit, treatment and/or diagnosis.

In another preferred embodiment, the apparatus 100 or the central processing computer 10 can also be programmed to identify the individual-specific or patient-specific educational information or individual-specific or patient-specific instructional information regarding the diagnosis, the treatment, the treatment plan, a prescribed electronic interactive activity or prescribed electronic interactive activities, and/or any information, link(s), or hyperlink(s) relating thereto or pertaining thereto.

In another preferred embodiment, the claim form or the claim submission can be date-stamped and/or time-stamped so that the claim processing corresponding to same can be tracked or monitored so as to facilitate audits of the insurer or payer or insurance company in order to ascertain if the insurer or payer or insurance company is properly and/or efficiently handling a claim or claims for the individual or patient and/or if the insurer or payer or insurance company is in compliance with any laws, rules, or regulations, governing claims processing and/or handling. Information regarding the date stamped and/or time stamped claim forms or claims, including the insurer's or the payer's, or the insurance company's, processing or handling of same, and the response or reply to same, can also be stored in the individual's or the patient's electronic healthcare record in the database 10H of the central processing computer 10, and/or in the database 10H of the central processing computer 10, and can be accessed and/or obtained by any authorized user or entity. In this manner, the apparatus 100 of the present invention can also be used in order to ascertain information regarding the processing and/or handling of claims or claim forms by a respective insurer or payer or insurance company for any individual or patient, or for any group or groups of individuals or patients. The operation of the apparatus 100 will then cease at step 1412.

In any and/or all of the embodiments described herein, the apparatus 100 and method of the present invention can be utilized in conjunction with electronic interactive activities of any type or kind in order to diagnose and/or to treat illnesses, sicknesses, injuries, conditions, syndromes, states of dysfunction, and/or any healthcare or healthcare-related problems or impairments, for individuals or patients of all ages.

In this regard, the apparatus 100 and method of the present invention can be utilized in order to provide an electronic interactive activity or electronic interactive activities via an electronic healthcare record or an electronic healthcare file and can be utilized by individuals and patients of any and/or all ages or age groups in order to provide for the use of an electronic interactive activity or electronic interactive activities which can be utilized in performing diagnostic evaluations or analyses, diagnoses, treatments, treatment planning, therapy, rehabilitation, training, or re-training, for and/or regarding any illnesses, sicknesses, injuries, conditions, syndromes, states of dysfunction, and/or any healthcare or healthcare-related problems or impairments, including, but not limited to, developmental, cognitive, physical, mental, behavioral, motor, speech, hearing, seeing, learning, or any other, disorders, illnesses, sicknesses, injuries, conditions, syndromes, or states of dysfunction, Alzheimer's disease, Dementia, learning disorders, attention deficit disorders, learning disabilities, muscular coordination disorders, chronic pain conditions, developmental disorders, speech disorders, cognitive disorders, mental acuity disorders, spatial disorders, temporal disorders, psychological disorders, physiological disorders, post traumatic stress disorders, lazy eye, atrophies of any kind or type, conditions or disorders that affect spatial and/or temporal conditions, muscular coordination and/or any other type of coordination or activity, or any other healthcare or healthcare-related disorder, illness, sickness, injury, condition, syndrome, or state of dysfunction.

In any and/or all of the embodiments described herein, the apparatus 100 of the present invention and/or the central processing computer 10 can be programmed to automatically generate an electronic interactive activity message(s) containing information regarding an availability of an electronic interactive activity or of an availability of an update to an electronic interactive activity. The central processing computer 10 can also automatically transmit the electronic interactive activity message to a provider communication device 20 of or associated with a provider of an individual or patient, the a payer communication device 30 of or associated with an insurer or a payer of an individual of or a patient, to a user or patient communication device 40 of or associated with an individual or a patient, or a caregiver of the individual or the patient, or to an intermediary communication device 50 of or associated with any intermediary.

In any and/or all of the embodiments described herein, the apparatus 100 of the present invention and/or the central processing computer 10 can also be programmed to automatically generate a prescribed or recommended electronic interactive activity reminder message(s) containing information for reminding an individual or a patient, or his or her caregiver, or his or her provider, to access, engage in, use, perform, or participate in, any prescribed or recommended electronic interactive activity. The central processing computer 10 can also automatically transmit the electronic interactive activity reminder message to a provider communication device 20 of or associated with a provider of an individual or patient, the a payer communication device 30 of or associated with an insurer or a payer of an individual of or a patient, to a user or patient communication device 40 of or associated with an individual or a patient, or a caregiver of the individual or the patient, or to an intermediary communication device 50 of or associated with any intermediary.

In any and/or all of the embodiments described herein, any of the herein-described reports, plans, messages, or data and/or information, described herein as being transmitted from one computer or communication device to another computer or communication device, can be transmitted as, in, or attached to, an e-mail message, a text message, an SMS message, or any electronic transmission or electronic data transmission, and/or can be transmitted directly to a computer or communication device or can be transmitted indirectly via, such as, for example, but not limited to, an e-mail server, e-mail servers, or any other intermediate computer or communication device.

In another preferred embodiment as well any and/or all of the embodiments described herein, the apparatus 100 and method of the present invention, and/or the central processing computer 10, can process electronic commerce transactions regarding or involving the purchase and/or sale of any electronic interactive activities and/or any equipment, goods, or products, relating thereto to used in connection therewith.

The apparatus and method of the present invention can also be utilized to generate and/or transmit a reminder message, an alert message, or a notification message, to an individual, a patient, or a caregiver of same, containing information regarding the availability of an electronic interactive activity or electronic interactive activities, to generate and/or transmit a reminder message, an alert message, or a notification message, to an individual, a patient, or a caregiver of same, regarding a time or a scheduled time of a prescribed time when an individual or a patient is to engage in performing or participating in an electronic interactive activity or electronic interactive activities, or to generate and/or transmit a reminder message, an alert message, or a notification message, to a healthcare provider, any other provider, or a caregiver, regarding an individual's or a patient's engagement in or use of, and/or the individual's or the patient's failure to engage in or failure to use, an electronic interactive activity or electronic interactive activities pursuant to a prescribed treatment or treatment plan, and/or including any data, information, or results, of or obtained from the individual's or a patient's engagement in or use of an electronic interactive activity or electronic interactive activities.

In another embodiment, in the case of use of the apparatus 100 and method of the present invention in connection with social networks, any and/or all social networking support group members, and/or individuals or patients invited to participate in a social networking support group, can each be verified, by utilizing the data and/or information stored in the database 10H and/or in any and/or all of the electronic healthcare records, electronic medical records, electronic dental records, electronic pharmacy records, and/or electronic behavioral health records which are utilized in, or in connection with, the apparatus 100, as having been diagnosed with the same or a similar illness, sickness or condition, or as having been prescribed the same or a similar treatment or procedure, or as being prescribed the same or similar treatment plan, so as to maintain compliance with all healthcare and/or other privacy laws, rules, and regulations and other concerns as well as ensuring the integrity of the social networking support group.

In any and/or all of the embodiments described herein, any and/or all of the any electronic healthcare records, electronic medical records, electronic dental records, electronic pharmacy records, electronic behavioral health records, utilized in connection with the apparatus 100 can contain a link(s) or a hyperlink(s) to any of the herein-described healthcare records computers 60, insurance exchange computers 80, social networking computers 90, and/or the media computers 95. In any and/or all of the embodiments described herein, any user, individual, patient, or caregiver, can use his or her user or patient communication device 40 to access any healthcare records computer 60 via his or her electronic healthcare record(s), electronic medical record(s), electronic dental record(s), electronic pharmacy record(s), or electronic behavioral health record(s) for any reason and/or purpose.

In any and/or all of the embodiments described herein, any user, individual, patient, or caregiver, can use his or her user or patient communication device 40 to access any insurance exchange computers 80 via his or her electronic healthcare record(s), electronic medical record(s), electronic dental record(s), electronic pharmacy record(s), or electronic behavioral health record(s) in order to purchase an insurance policy, program, or service for any healthcare insurance, life insurance, disability insurance, automobile insurance, property insurance, liability insurance, or any other kind or type of insurance, to submit insurance claims or payment requests pursuant to any healthcare insurance, life insurance, disability insurance, automobile insurance, property insurance, liability insurance, or any other kind or type of insurance, or to check the status of any insurance claims or payment requests.

In any and/or all of the embodiments described herein, any user, individual, patient, or caregiver, can use his or her user or patient communication device 40 to access any social networking computers 90 via his or her electronic healthcare record(s), electronic medical record(s), electronic dental record(s), electronic pharmacy record(s), or electronic behavioral health record(s) in order to engage or participate in any social networking activity, support groups, on-line chats or discussions, seminars, or any other activity which one can engage in on, in, or via a social network.

In any and/or all of the embodiments described herein, any user, individual, patient, or caregiver, can use his or her user or patient communication device 40 to access any media computers 95 via his or her electronic healthcare record(s), electronic medical record(s), electronic dental record(s), electronic pharmacy record(s), or electronic behavioral health record(s) in order to search for and/or obtain any information provided by the respective media computer 95. The apparatus 100 and method of the present invention can also be used in any applications wherein the healthcare of groups of individuals is monitored and/or observed and/or managed by a person or entity having such a responsibility, role, or interest.

In any and/or all of the embodiments described herein, the apparatus 100 and method of the present invention can also be utilized in a same, similar, or analogous manner, in order to process and/or to provide veterinary healthcare information and/or veterinary healthcare-related information for and regarding any kind or type of animal or animals or any type or kind of pet or pets.

In any and/or all of the embodiments described herein, the apparatus 100 and method of the present invention can also be utilized as a clearinghouse for facilitating the offering, selling, buying, trading, and/or other commerce and/or transactions, involving healthcare and/or healthcare-related services, products and/or goods.

While the present invention has been described and illustrated in various preferred and alternate embodiments, such descriptions are merely illustrative of the present invention and are not to be construed to be limitations thereof. In this regard, the present invention encompasses all modifications, variations and/or alternate embodiments, with the scope of the present invention being limited only by the claims which follow.

What is claimed is:

1. An apparatus, comprising:
a user communication device, wherein the user communication device is associated with an individual, and further wherein the user communication device further comprises:
a first processor;
a first display;
a first input device;
a first video recording device; and
a first microphone or a first audio recording device;
a provider communication device, wherein the provider communication device is associated with a healthcare provider of the individual, and further wherein the provider communication device further comprises:
a second processor;
a second display;
a second input device;
a second video recording device; and
a second microphone or a second audio recording device; and
a computer, wherein the computer is specially programmed to maintain, and to process information for and regarding, an electronic healthcare record of or for the individual, and further wherein the computer is specially programmed to administer or to provide a video game to the user communication device via or in connection with the electronic healthcare record of or for the individual and to allow the healthcare provider to observe an activity or activities of the individual during the individual's interaction with, or playing of, the video game, wherein the computer further comprises:
a database, wherein the database stores the electronic healthcare record of or for the individual and stores the video game;
a third processor, wherein the third processor is specially programmed to process information regarding the electronic healthcare record of or for the individual and is specially programmed to administer or to provide the video game to the user communication device and to record information or results regarding the individual's interaction with, or playing of, the video game, wherein the video game is administered or provided pursuant to a diagnostic evaluation or a diagnostic analysis of the individual or the video game is administered or provided pursuant to a treatment of the individual; and
a transmitter,
wherein the computer processes a request to access the video game, wherein the request to access the video game is transmitted from the user communication device, and further wherein the video game is accessed via the electronic healthcare record of or for the individual, wherein the computer administers or provides the video game to the individual via the user communication device, and further wherein the computer records and stores, in the electronic healthcare record of or for the individual, information regarding the individual's activity during, or interactions with, the video game, and further wherein the computer provides the video game to the provider communication device for allowing the healthcare provider to observe or monitor the individual's actions or interactions in or during the video game via the second display, and further wherein notes or observations entered into the provider communication device regarding the individual or actions of the individual during the providing of the video game are transmitted from the provider communication device to the computer, and further wherein the computer stores the notes or observations in the electronic healthcare record of or for the individual,
wherein the first video recoding device records a first video or a first video clip, the first microphone or the first audio recording device records a first audio or a first audio clip, the second video recoding device records a second video or a second video clip, or the second microphone or the second audio recording device records a second audio or a second audio clip, and further wherein the computer receives and stores, in the electronic healthcare record of or for the individual, the first video or the first video clip, the first audio or the first audio clip, the second video or the second video clip, or the second audio or the second audio clip.

2. The apparatus of claim 1, wherein the computer provides the video game to the provider communication device in real-time.

3. The apparatus of claim 1, wherein the computer records, and stores a recording of, the video game and the individual's activity during, or interactions with, the video game, and further wherein the computer stores the recording in the electronic healthcare record of or for the individual.

4. The apparatus of claim 3, wherein the computer provides the recording to the provider communication device or to a second provider communication device.

5. The apparatus of claim 4, wherein the computer receives the notes or the observations entered into the provider communication device and transmitted to the computer, or receives second notes or second observations entered into the second provider communication device and transmitted to the computer, and further wherein the computer stores the notes or the observations or stores the second notes or the second observations in the electronic healthcare record of or for the individual.

6. The apparatus of claim 1, wherein the video game is administered or provided pursuant to a diagnostic evaluation or a diagnostic analysis of the individual.

7. The apparatus of claim 1, wherein the video game is administered or provided pursuant to a treatment of the individual.

8. The apparatus of claim 1, wherein the computer processes information for performing a diagnostic evaluation of or for the individual.

9. The apparatus of claim 8, wherein the computer generates a diagnostic report containing information regarding a diagnosis or a list of possible diagnoses regarding the individual or containing information regarding an electronic interactive activity or a second video game prescribed for performing a further diagnostic evaluation for the individual, and further wherein the computer stores the diagnostic report in the electronic healthcare record of or for the individual.

10. The apparatus of claim 1, wherein the computer processes information for generating a treatment report containing a prescribed treatment for a diagnosis of the individual, and further wherein the computer stores the treatment report in the electronic healthcare record of or for the individual.

11. The apparatus of claim 10, wherein the computer generates a treatment plan containing information regarding an electronic interactive activity or a second video game prescribed for treating the individual, and further wherein the computer stores the treatment plan in the electronic healthcare record of or for the individual.

12. The apparatus of claim 1, wherein the computer generates an insurance claim or a claim for payment upon completion of the video game, and further wherein the computer transmits the insurance claim or the claim for payment, to a third communication device, wherein the third communication device is associated with a healthcare insurer or a healthcare payer.

13. The apparatus of claim 1, wherein the user communication device is a wireless device, a cellular telephone, a personal digital assistant, a smart phone, a tablet, or a tablet computer.

14. The apparatus of claim 1, wherein the video game is provided by the computer to the user communication device via the Internet or the World Wide Web.

15. The apparatus of claim 9, wherein the electronic interactive activity is a puzzle activity.

16. The apparatus of claim 9, wherein the electronic interactive activity is an audio game.

17. The apparatus of claim 9, wherein the electronic interactive activity is an electronic exercise or a digital exercise.

18. The apparatus of claim 9, wherein the electronic interactive activity is a questionnaire or a survey.

19. The apparatus of claim 12, wherein the insurance claim or the claim for payment contains a link or a hyperlink to the electronic healthcare record of or for the individual.

20. The apparatus of claim 12, wherein the insurance claim or the claim for payment contains a link or a hyperlink to the notes or the observations entered into the provider communication device regarding the individual or actions of the individual during the providing of the video game.
